(19) United States
(12) Patent Application Publication
(10) Pub. No.: US 2022/0111039 A1
(43) Pub. Date: Apr. 14, 2022

(54) IMMUNOGENIC CONSTRUCTS, COMPOSITIONS, AND METHODS FOR INDUCING IMMUNE RESPONSE

(71) Applicants: Oregon Health & Science University, Portland, OR (US); PDX Pharmaceuticals, Inc., Portland, OR (US)

(72) Inventors: Wassana Yantasee, Lake Oswego, OR (US); Sherif Reda, Portland, OR (US); Moataz Reda, Portland, OR (US); Worapol Ngamcherdtrakul, Portland, OR (US); Ruijie Wang, Portland, OR (US)

(73) Assignees: Oregon Health & Science University, Portland, OR (US); PDX Pharmaceuticals, Inc., Portland, OR (US)

(21) Appl. No.: 17/552,331

(22) Filed: Dec. 15, 2021

Related U.S. Application Data
(63) Continuation of application No. PCT/US2021/041487, filed on Jul. 13, 2021.
(60) Provisional application No. 63/051,351, filed on Jul. 13, 2020.

(51) Int. Cl. A61K 39/215; A61K 39/00

(52) U.S. Cl. CPC A61K 39/215; A61K 2039/55561; A61K 2039/6093

(57) ABSTRACT

Disclosed are immunogenic constructs including: a nanoparticle; a cationic polymer electrostatically bound to an exterior surface of the nanoparticle and a stabilizer bound to the cationic polymer or the exterior surface of the nanoparticle; and an antigen or antigen producing agent. Optionally, the constructs may include adjuvant and/or one or more functional oligonucleotide(s) (e.g., siRNA or pDNA). Also disclosed are methods of using the provided immunogenic constructs for co-delivering an adjuvant, antigen, and optionally siRNA to a cell, inducing immune response in a subject, and treating or preventing an infectious disease in a subject.

### CROSS-REFERENCE TO RELATED APPLICATION

This application claims is a continuation of co-pending International Application PCT/US2021/041487, filed Jul. 13, 2021, which claims priority to and the benefit of the earlier filing date of U.S. Provisional Application No. 63/051,351, filed Jul. 13, 2020; these earlier applications are incorporated by reference herein in their entirety.

### STATEMENT REGARDING FEDERALLY SPONSORED RESEARCH

This invention was made with government support under grant number R44 CA217534 from the National Institutes of Health. The government has certain rights to the invention.

### BACKGROUND

During the first weeks of 2020, the world has evidenced the emergence of a new human pathogen that achieved enough zoonotic spillover to cause a pandemic, from a highly pathogenic beta-coronavirus. The 2019 novel Coronavirus (SARS-CoV-2) that is the cause of the highly infectious disease known as COVID-19 is a new member of the beta-coronaviruses, which includes the Severe Acute Respiratory Syndrome Coronavirus (SARS-CoV-1) that caused epidemics in China in 2002-2003 and the Middle East Respiratory Syndrome (MERS-CoV) that affected Saudi Arabia and neighbor countries in 2012-2013.

After initially emerging in China, the rapid spread of COVID-19 has resulted in >13 million confirmed cases and over half a million deaths (as of July 2020). There is an urgent need for safe and effective therapeutic and prophylactic agents against this novel virus.

Accordingly, there is a need for a new immunogenic strategy that is applicable for this virus and for emerging infectious diseases.

### SUMMARY OF THE DISCLOSURE

The disclosure provides immunogenic constructs, compositions, and methods for inducing immune responses against infectious agents. For example, compositions of the disclosure can be used to induce an immune response against beta-coronavirus infections, e.g., infections by SARS-CoV-2, SARS-CoV-1, MERS-CoV, and related viruses. The disclosed technology is also adaptable to other current infectious diseases, such as Dengue fever, malaria, along with infectious diseases in animals (such as pets and livestock).

In one aspect, the disclosure features an immunogenic construct that contains a nanoparticle, a crosslinked cationic polymer bound to an exterior surface of the nanoparticle, a stabilizer bound to the crosslinked cationic polymer or the exterior surface of the nanoparticle, and an antigen (e.g., a full-length protein, a protein subunit, a polypeptide, a peptide, or a mixture thereof) or antigen producing agent (such as an antigen producing nucleic acid, e.g., mRNA or pDNA) for an infectious agent.

In some embodiments, the immunogenic construct further includes an adjuvant. In some embodiments, the adjuvant includes one or more of a CpG oligonucleotide, a DNA TLR agonist containing a CpG sequence, a non-CpG DNA TLR agonist, an RNA TLR agonist, an aluminum salt, an anti-CD40 antibody, a fusion protein, a cytokine, a small molecule TLR agonist, an oil- or surfactant-based adjuvant, a lipopolysaccharide, a plant extract, or a derivative thereof. In some embodiments, the adjuvant includes a CpG oligonucleotide (e.g., CpG ODN 1826 or CpG ODN 7909/2006). In some embodiments, the adjuvant includes poly I:C. In some embodiments, the adjuvant is present at 1-20 wt. % of the nanoparticle platform (NP or polymer/stabilizer coated nanoparticles) (e.g., 1-10 wt. %, 2-7 wt. %, 2-4 wt. %, 2-10 wt. %, 5-10 wt. %, 10-20 wt. %.; or about 4 wt. %, about 5 wt. %, about 6 wt. %, about 7 wt. %, about 10 wt. %, or about 20 wt. %). In some embodiments, the adjuvant is present at 2-10 wt. % of the NP.

In some embodiments, the nanoparticle is a silica nanoparticle (e.g., a mesoporous silica nanoparticle), a silicon nanoparticle, an iron oxide nanoparticle, a gold nanoparticle, a silver nanoparticle, a carbon nanoparticle, or a carbon nanotube. The pore size of the mesoporous nanoparticle in various embodiments is 2, 3, 4, 5, 6, 7, 8, 9, 10, 2-5, 2-7, 6-10, 11-15, 16-20, 21-30, or 31-50 nm.

In embodiments, the nanoparticle is an adjuvant nanoparticle or an immunostimulatory nanoparticle (e.g., a liposome, a lipoplex particle, a lipid-based particle, a polyplex particle, a polymer-based particle, an inorganic particle (e.g., a calcium phosphate or carbonate nanoparticle, an aluminum salt particle, a silica particle), a virosome or a virus-like particle, or a nanoparticle comprising one or more of 1,2-dioleoyl-3-trimethylammonium propane (DOTAP), cholesterol, 3β-[N—(N′,N′-dimethylaminoethane)-carbamoyl] cholesterol, phosphatidylcholine/cholesterol, chitosan, poly-γ-glutamic acid (γ-PGA), hyaluronic acid, polyethylenimine (PEI), poly(propylacrylic acid), polypropylene sulfide, poly(lactic-co-glycolic acid) (PLGA), amylopectin, maltodextrin, polystyrene, gold, cobalt oxide, alum, tri-palmitoyl-S-glyceryl cysteine (PAM₃Cys), squalene, Montanide ISA 50V, Montanide ISA 51, Montanide ISA 201, Montanide ISA 206, and Montanide ISA 720).

In some embodiments, the cationic polymer is selected from the group consisting of polyethylenimine (PEI), chitosan, polypropylenimine, polylysine, polyamidoamine, poly(allylamine), poly(diallyldimethylammonium chloride), poly(N-isopropyl acrylamide-co-acrylamide), poly(N-isopropyl acrylamide-co-acrylic acid), diethylaminoethyl-dextran, poly-(N-ethyl-vinylpyridinium bromide), poly(dimethylamino)ethyl methacrylate, and poly(ethylene glycol)-co-poly(trimethylaminoethylmethacrylate chloride). In some embodiments, the cationic polymer is PEI. In some embodiments, the cationic polymer has a molecular weight of about 0.8 kDa to about 25 kDa (e.g., about 0.8 kDa to about 10 kDa, about 0.8 kDa to about 5 kDa, about 0.8 kDa to about 2.5 kDa, about 2.5 kDa to about 10 kDa, or about 5 kDa to about 10 kDa). In some embodiments, the cationic polymer is present at about 1 to 50 wt. % of the NP (e.g., 5 to 40 wt. %, 10 to 30 wt. %, 20 to 30 wt. %, 5 to 10 wt. %, 5 to 15 wt. %, 5 to 20 wt. %, 5 to 25 wt. %, 5 to 30 wt. %, 10 to 20 wt. %, 10 to 25 wt. %, or 25 to 40 wt. %; or about 5, 10, 15, 20, 25, 30, or 35 wt. %). In some embodiments, the cationic polymer is present at 10 to 20 wt. % of the NP.

In some embodiments, the stabilizer is selected from the group consisting of polyethylene glycol (PEG), dextran, polysialic acid, hyaluronic acid, polyvinyl pyrrolidone, polyvinyl alcohol, and polyacrylamide. In some embodiments, the stabilizer is the PEG. In some embodiments, the stabilizer has a molecular weight of from about 1 kDa to about 20 kDa (e.g., about 0.8 kDa to about 10 kDa, about 0.8 kDa to about 5 kDa, about 2 kDa to about 10 kDa, about 0.8 kDa to about 2.5 kDa, about 2.5 kDa to about 10 kDa, or about 5 kDa to about 10 kDa). In some embodiments, the stabilizer is present at 1 to 50 wt. % of the NP (e.g., 5 to 30 wt. %, 10 to 20 wt. %, 10 to 25 wt. %, 5 to 15 wt. %, 5 to 20 wt. %, 5 to 25 wt. %, or 1 to 10 wt. %, or about 5, 10, 15, 20, 25, 35, 40 or 45 wt. %). The stabilizer may be introduced before or after cargo loading, or both.

In some embodiments, the infectious agent is a virus, such as a beta-coronavirus (e.g., SARS-CoV-2, SARS-CoV-1, or MERS-CoV). In some embodiments, the antigen is a recombinant full-length protein, e.g., a full-length SARS-CoV-2 spike glycoprotein, a SARS-CoV-2 nucleocapsid protein, or a SARS-CoV-2 membrane protein. In some embodiments, the antigen is a protein subunit, e.g., a protein subunit that corresponds to the S1, S2, or Receptor Binding Domain (RBD) region of the SARS-CoV-2 spike glycoprotein. In some embodiments, the antigen is a peptide or a mixture of peptides that correspond to an immunogenic sequence of an infectious agent. For example, the infectious agent is SARS-CoV-2, and the antigen has the peptide(s) sequence of SEQ ID NO: 1, 2, 3, 4, 5, 6, 7, and/or 8. In some embodiments, the antigen producing agent is an mRNA or a pDNA, e.g., an mRNA or a pDNA that is expressed or translated into an antigen in vitro (e.g., DC), or in vivo (e.g., DC, muscle cells). In some embodiments, the antigen or antigen producing agent is present at 0.5-20 wt. % of the NP (e.g., 0.5-10 wt. %, 1-6 wt. %, 1-15 wt. %, 1.5-10 wt. %, or 2-5 wt. %). In some embodiments, the antigen may include of a mixture of protein subunits and peptides.

In some embodiments, the immunogenic construct includes at least one type of oligonucleotide selected from siRNA, miRNA, miRNA mimic, or antisense oligonucleotide. In some embodiments, the at least one type of oligonucleotide is electrostatically bound to the cationic polymer. In some embodiments, the at least one type of oligonucleotide includes a siRNA, e.g., one that inhibits or downregulates a gene associated with immunosuppression of a cell, such as an antigen-presenting cell (e.g., a dendritic cell or a macrophage). In some embodiments, the gene is STAT3, IDO-1, IL-6, or PD-L1. In some embodiments, the at least one type of oligonucleotide is present at about 1-50 wt. % of the NP (e.g., 2 wt. %, 3 wt. %, 4 wt. %, 5 wt. %, 2-5 wt. %, 2-8 wt. %, 2 to 10 wt. %, 2 to 25 wt. %, or 2 to 50 wt. %).

In some embodiments, the immunogenic construct further includes a targeting agent for a cell, such as an antigen-presenting cell (e.g., a dendritic cell or a macrophage). In some embodiments, the targeting agents is mannose, monoclonal or polyclonal antibodies or fragments thereof that recognize and bind to epitopes displayed on the surface of the antigen-presenting cell, aptamers, and ligands that bind to surface receptors on the antigen-presenting cell. In some embodiments, the targeting agent is present at 0.1-20 wt. % of the NP (e.g., 0.1 to 1 wt. %, 0.2 to 2 wt. %, 1 to 5 wt. %, or 1 to 10 wt. %; or about 1, 2, 3, 4, 5, 6, 7, 8, or 9 wt. %).

In some embodiments, the immunogenic construct further includes a labeling agent. In some embodiments, the labeling agent is a fluorescent dye and/or a metal probe (e.g., a lanthanide probe, a quantum dot, a gold nanoparticle, or a gadolinium chelate).

In some embodiments, the immunogenic construct has a hydrodynamic diameter of about 10 nm to about 999 nm (e.g., about 80 nm to about 200 nm, or about 90 nm to about 130 nm), measured in aqueous solution (such as PBS, Tris buffer, or water) by a dynamic light scattering technique or a Zetasizer (Malvern Panalytical) or similar device) In some embodiments, the immunogenic construct has a hydrodynamic diameter of about 1 micron to about 10 microns (e.g., about 1 micron to about 2 microns) measured in aqueous solution (such as PBS, Tris buffer, or water). In some embodiments, the nanoparticle has a diameter of about 5 nm to 999 nm (e.g., about 20 nm to about 200 nm, about 30 nm to about 60 nm, about 10 nm, about 20 nm, about 30 nm, about 50 nm, about 60 nm, about 200 to about 750 nm or about 500 to 999 nm), e.g., as measured by transmission electron microscopy.

The disclosure further features an immunogenic construct that contains a nanoparticle, a lipid layer, and an antigen (e.g., full-length protein, protein subunit, polypeptide, or a peptide) or antigen producing agent (such as an antigen producing nucleic acid, e.g., mRNA or pDNA) for an infectious agent.

In some embodiments, the immunogenic construct further contains an adjuvant. In some embodiments, the adjuvant includes one or more of a CpG oligonucleotide, a DNA TLR agonist containing a CpG sequence, a non-CpG DNA TLR agonist, an RNA TLR agonist, an aluminum salt, an anti-CD40 antibody, a fusion protein, a cytokine, a small molecule TLR agonist, an oil- or surfactant-based adjuvant, a lipopolysaccharide, a plant extract, or a derivative thereof. In some embodiments, the adjuvant includes a CpG oligonucleotide (e.g., CpG ODN 1826 or CpG ODN 7909/2006). In some embodiments, the adjuvant is loaded into the NP. In some embodiments, the adjuvant is loaded on or within the lipid layer. In some embodiments, the adjuvant is present at 1-20 wt. % of the NP (e.g., 1-10 wt. %, 2-7 wt. %, 2-4 wt. %, 2-10 wt. %, 5-10 wt. %, 10-20 wt. %.; or about 4 wt. %, about 5 wt. %, about 6 wt. %, about 7 wt. %, about 10 wt. %, or about 20 wt. %). In some embodiments, the adjuvant is present at 2-10 wt. % of the NP.

In some embodiments, the nanoparticle is a silica nanoparticle (e.g., a mesoporous silica nanoparticle), a silicon nanoparticle, an iron oxide nanoparticle, a gold nanoparticle, a silver nanoparticle, or a carbon nanotube.

In some embodiments, the nanoparticle is an adjuvant nanoparticle or an immunostimulatory nanoparticle (e.g., a liposome, a lipoplex particle, a lipid-based particle, a polyplex particle, a polymer-based particle, an inorganic particle (e.g., a calcium phosphate nanoparticle, an aluminum salt particle, a silica particle), a virus-like particle, or a nanoparticle comprising one or more of 1,2-dioleoyl-3-trimethylammonium propane (DOTAP), cholesterol, 3β-[N—(N′,N′-dimethylaminoethane)-carbamoyl] cholesterol, phosphatidylcholine/cholesterol, chitosan, poly-γ-glutamic acid (γ-PGA), hyaluronic acid, polyethylenimine (PEI), poly(propylacrylic acid), polypropylene sulfide (PPS), poly(lactic-co-glycolic acid) (PLGA), amylopectin, maltodextrin, polystyrene, gold, cobalt oxide, alum, tri-palmitoyl-S-glyceryl cysteine (PAM₃Cys), squalene, Montanide ISA 50V, Montanide ISA 51, Montanide ISA 201, Montanide ISA 206, and Montanide ISA 720).

In some embodiments, the lipid layer is a monolayer or multilayer membrane comprising one or more of lipids selected from a neutral lipid (e.g., a prostaglandin, an eicosanoid, or a glyceride), a fatty-acid-modified lipid (e.g., 2-diphytanoyl-sn-glycero-3-phosphocholine or 1-(12-biotinyl(aminododecanoyl))-2-oleoyl-sn-glycero-3-phosphoethanolamine), a phospholipid (e.g., phosphatidylcholine, phosphatidylethanolamine, 1,2-distearoyl-sn-glycero-3-phosphochline, or 1,2-dioleoyl-sn-glycero-3-phosphoethanolamine), a fatty acid (e.g., stearic acid or lauric acid), a polymerizable lipid (e.g., cholesterol-PEG or distearoyl-rac-glycerol-PEG2K), a cationic lipid (e.g., 1,2-dioleoyl-3-trimethylammonium-propane or dimethyldioctadecylammonium bromide), a sphingolipid (e.g., a sphingomyelin or a ceramide), and a sterol (e.g., a cholesterol or a stifmasterol). In some embodiments, the lipid layer comprises 1,2-dioleoyl-sn-glycero-3-phosphoethanolamine, dimethyldioctadecylammonium bromide, cholesterol, 1,2-disteraoyl-sn-glycero-3-phosphocholine, and distearoyl-rac-glycerol-PEG2K. In embodiments, the lipid layer is present at 0.1-99.9 wt. % of the NP.

In some embodiments, the infectious agent is a virus, such as a beta-coronavirus (e.g., SARS-CoV-2, SARS-CoV-1, or MERS-CoV). In some embodiments, the antigen is a recombinant full-length protein, e.g., a full-length SARS-CoV-2 spike glycoprotein, a SARS-CoV-2 nucleocapsid protein, or a SARS-CoV-2 membrane protein. In some embodiments a combination of antigens (e.g., 1, 2, 3, 4, 5, 6, 7, 8, 9, 10, or more different antigens) are used in a formulation. In some embodiments, the antigen is a protein subunit, e.g., a protein subunit that corresponds to the S1, S2, or RBD region of the SARS-CoV-2 spike glycoprotein. In some embodiments, the antigen is a peptide or a mixture of peptides that correspond to an immunogenic sequence of an infectious agent. For example, the infectious agent is SARS-CoV-2, and the antigen has the peptide(s) sequence of SEQ ID 1, 2, 3, 4, 5, 6, 7, and/or 8. In some embodiments, the antigen producing agent is an mRNA or a pDNA, e.g., an mRNA or a pDNA that is expressed or translated into an antigen in vitro (e.g., DC), or in vivo (e.g., DC, muscle cells). In some embodiments, the antigen or antigen producing agent is present at 0.5-20 wt. % of the NP (e.g., 1-15 wt. %, 1.5-10 wt. %, or 2-5 wt. %). In some embodiments, the antigen may comprise of a mixture of protein subunits and peptides.

In some embodiments, the infectious agent is a bacterium. In some embodiments, the antigen is a toxoid, e.g. inactivated toxin intended to immunize against a certain bacterial toxin. In some embodiments, the antigen is a polysaccharide of the bacteria intended to create immunity against the sugar coating of the bacteria. In some embodiments, the antigen consists of one or more recombinant protein(s) from the bacteria.

In some embodiments, the immunogenic construct includes at least one type of oligonucleotide selected from siRNA, miRNA, miRNA mimic, or antisense oligonucleotide. In some embodiments, the at least one type of oligonucleotide includes a siRNA, e.g., one that inhibits or downregulates a gene associated with immunosuppression of a cell, such as an antigen-presenting cell (e.g., a dendritic cell or a macrophage). In some embodiments, the gene is STAT3, IDO-1, IL-6, or PD-L1. In some embodiments the at least one type of oligonucleotide is loaded into the NP. In some embodiments, the at least one type of oligonucleotide is loaded on or within the lipid layer. In some embodiments, the at least one type of oligonucleotide is present at 0.01 to 10 wt. % of the NP.

In some embodiments, the immunogenic construct further includes a targeting agent for a cell, such as an antigen-presenting cell (e.g., a dendritic cell or a macrophage). In some embodiments, the targeting agents is mannose, a monoclonal or polyclonal antibody or a fragment thereof that recognizes and binds to an epitope displayed on the surface of the antigen-presenting cell, an aptamers, or a ligand that binds to a surface receptor on the antigen-presenting cell.

In some embodiments, the immunogenic construct further includes a labeling agent. In some embodiments, the labeling agent is a fluorescent dye and/or a metal probe (e.g., a lanthanide probe, a quantum dot, a gold nanoparticle, or a gadolinium chelate).

In some embodiments, the immunogenic construct has a hydrodynamic diameter of 10 nm to 10 microns. In some embodiments, the immunogenic construct has a hydrodynamic diameter of about 10 nm to about 999 nm (e.g., about 80 nm to about 200 nm, or about 90 nm to about 150 nm), measured in aqueous solution (such as PBS, Tris buffer, or water). In some embodiments, the immunogenic construct has a hydrodynamic diameter of about 1 micron to about 10 microns (e.g., about 1 micron to about 2 microns) measured in aqueous solution (such as PBS, Tris buffer, or water). In some embodiments, the nanoparticle has a diameter of about 5 nm to 999 nm (e.g., about 20 nm to about 200 nm, about 30 nm to about 60 nm, about 200 to about 750 nm or about 500 to 999 nm), e.g., as measured by transmission electron microscopy.

In some embodiments, the nanoparticle is an antioxidant nanoparticle.

In another aspect, the disclosure features a pharmaceutical composition including an immunogenic construct of the disclosure and a pharmaceutically acceptable excipient. In some embodiments, the pharmaceutical composition further includes an unbound adjuvant as described.

In another aspect, the disclosure features a vaccine including an immunogenic construct of the disclosure and a pharmaceutically acceptable excipient.

In another aspect, the disclosure features a method of co-delivering an oligonucleotide (e.g., siRNA), an antigen or antigen producing agent (e.g., mRNA or pDNA), and/or an adjuvant to a cell (e.g., a muscle cell or an antigen-presenting cell such as a dendritic cell or a macrophage). The method includes contacting the cell with an immunogenic construct of the disclosure. In some embodiments, the immunogenic construct includes at least one antigen producing agent (e.g., mRNA or pDNA) and is administered intramuscularly to a subject and is taken up by muscle cells, wherein the immunogenic construct induces the muscle cells to produce at least one antigen for subsequent immune cell activation.

In another aspect, the disclosure features a method of inducing an immune response against an infectious agent in a subject. The method includes administering to the subject an immunogenic amount of an immunogenic construct of the disclosure. In some embodiments, the subject is a human. In some embodiments, the subject is immunocompromised (e.g., an older or elderly subject, e.g., over 50, 55, 60, 65, 70, 75, or 80 years of age, or a subject with underlying medical condition(s) such as diabetes and cancer, known to be immunocompromised and susceptible to infection). In some embodiments, the immunogenic construct is administered by intramuscular injection.

In another aspect, the disclosure features a method of increasing immune response against an infectious agent in a subject. The method includes administering to the subject an effective amount of an immunogenic construct of the disclosure. In some embodiments, the subject is a human. In some embodiments, the subject is immunocompromised (e.g., an old or elderly subject, e.g., over 50, 55, 60, 65, 70, 75, or 80 years of age, or a subject with underlying medical condition(s) known to be immunocompromised and susceptible to infection). In some embodiments, the immunogenic construct is administered by intramuscular injection. In some embodiments, the immunogenic construct is administered by inhalation.

In another aspect, the disclosure features a method of vaccinating a subject against an infectious agent. The method includes administering to the subject an effective amount of an immunogenic construct of the disclosure. In some embodiments, the subject is a human. In some embodiments, the subject is immunocompromised (e.g., an older or elderly subject, e.g., over 50, 55, 60, 65, 70, 75, or 80 years of age, or a subject with underlying medical condition(s) known to be immunocompromised and susceptible to infection). In some embodiments, the immunogenic construct is administered by intramuscular injection. In some embodiments, the immunogenic construct is administered by inhalation.

### BRIEF DESCRIPTION OF THE DRAWINGS

FIG. 1 is a scheme demonstrating the use of immunogenic constructs, according to an embodiment of the disclosure, to induce an immune response against an infection (illustrated with viral infection with SARS-CoV-2, a.k.a. CoV). Upon intramuscular or subcutaneous injection, the immunogenic constructs (AIRISE-CoV) are taken up by antigen-presenting cells (APCs, e.g., dendritic cells and macrophages). Immune activation by CpG and the inhibition of immunosuppressive genes by siRNA in the APCs enhance their activities to process the delivered antigen for presentation (A). The activated antigen-loaded APCs travel from the injection site to lymph nodes (B) and subsequently activate antigen-specific CD8+ T cells (C), followed by their proliferation into effector and memory T cells against the virus. Activated APCs also activate B cells and CD4+ T cells, the latter of which can further activate CD8+ T cells and B cells, which, in turn, produce humoral immune response (antibodies, D) against the viral infection (current and future) everywhere in the body, such as the lungs (E). If the constructs deliver antigen producing agents (such as mRNA and pDNA), muscular cells will also take up the injected constructs and produce the antigens to be processed by the APCs, followed by the same processes A-E.

FIG. 2 is a graph showing the hydrodynamic sizes (diameters, or Z-average diameters) of mesoporous silica nanoparticles coated with PEI and PEG (nanoparticle platform; NP) loaded with SIINFEKL peptide (SEQ ID NO: 90; SF, Anaspec) and CpG 1826 (Invivogen).

FIG. 3 shows the hydrodynamic size of NPs (MSNP-PEI-PEG) loaded with poly I:C at about 2 wt. % and 9 wt. % of the NP, measured in PBS.

FIGS. 4A-4D show STAT3 knockdown at 48 hrs in multiple cells of multiple species using (FIG. 4A) NP loaded with siRNA against STAT3 (siSTAT3) and (FIG. 4B) NP loaded with siSTAT3 and CpG. D-17 (dog osteosarcoma), BMDC (Bone-marrow derived dendritic cells from mice), J774 (mouse macrophage), B16F10 (mouse melanoma), and HCC1954 (human breast cancer) qRT-PCR analysis for STAT3 and HPRT mRNA was performed with primers of corresponding species. A single siSTAT3 sequence was used throughout. siSCR=scrambled siRNA control. ***p<0.001; ****p<0.0001. Dose of siSTAT3 was 50 nM, and that of CpG was 220 nM. FIG. 4C is a graph showing that co-delivery of non-targeting scrambled siRNA (siSCR) and CpG by NP or Dharmafect to dendritic cells harvested from C3H/HEJ mice. Dose of each siRNA was 50 nM and at 2.0 wt. % of NP, and dose of CpG was 4 wt. % of NP. siRNA-Dharmafect formulation was prepared following the manufacturer's protocol. mRNA was analyzed with qRT-PCR at 48 h post-treatment. FIG. 4D is a graph showing that besides siSTAT3, NP can also deliver siRNA against PD-L1 (siPDL1) resulting in effective knock down of PD-L1 protein expression (as measured by flow cytometry) in LLC-JSP cells. The cells were treated with NP containing 30 nM siRNA against PD-L1 (siPDL1) or 30 nM scrambled siRNA (siSCR) at 2 wt. % siRNA. At 72 hr post treatment, cells were harvested and assessed for PD-L1 protein expression by flow cytometry. RFU=Relative fluorescence units. “NP” denotes mesoporous silica nanoparticles coated with cross-linked PEI and PEG as described in Ngamcherdtrakul et al., Advanced Functional Materials, 25(18):2646-2659, 2015 and U.S. Patent Application Publication No. 2017/0173169.

FIGS. 5A-5C show that siSTAT3-CpG-NP that induces immunogenic effect greater than NP delivering siSTAT3 or CpG alone. Mice having bilateral B16F10 melanoma tumors were injected intratumorally in one tumor only for a total of 3 doses at 3 days apart. Tumor growth curves of (FIG. 5A) local treated tumors and (FIG. 5B) distant untreated tumors are plotted as mean±SEM. (FIG. 5C) Survival curve of the mice. Dose (per each injection): 20 μg CpG; 4 μg siSTAT3; 0.22 mg NP. *p<0.05, ****p<0.0001 for CpG-NP vs. siSTAT3-CpG-NP.

FIGS. 6A-6C show siSTAT3-CpG-NP enhances proliferation of CD8⁺ T cells in tumors and draining lymph nodes (DLN) better than NP delivering siSTAT3 or CpG alone. Model, treatment dose, and schedule were as in FIGS. 5A-5C. 7 days after the first treatment, cells harvested from tumors and DLN of both local (treated) and distant (untreated) tumors were analyzed to determine the ratio of CD8⁺ T cells over CD4⁺FoxP3⁺ regulatory T cells in the live CD45+CD3⁺ T cell populations of tumors (FIG. 6A) and DLNs (FIG. 6B), along with effector (CD44⁺) CD8⁺ T cell's proliferation status (Ki-67) in the lymph nodes (FIG. 6C). *p<0.05, **p<0.01, ***p<0.001, ****p<0.0001 (n=3/group) for siSTAT3-CpG-NP vs. saline, unless the bracket specifies otherwise.

FIG. 7 is a graph showing the percentage of IFNγ activated CD8+ T cells after incubation in the presence of SF (SEQ ID NO: 90). The cells were obtained from untreated mice, mice treated with NPs loaded with SF and CpG (CpG-SF-NP), NPs loaded with SF (SF-NP), NP loaded with CpG (CpG-NP), and mice treated with SF formulated with Incomplete Freund's Adjuvant (IFA/SF). *p<0.05. Doses used: 16 μg CpG and 40 μg SF.

FIGS. 8A and 8B show (FIG. 8A) the hydrodynamic sizes of 1) mesoporous silica nanoparticles coated with PEI and PEG (NP) and loaded with about 3 wt. % SARS-CoV-2 spike protein, 2 wt. % siRNA, and 4 wt. % CpG; and (FIG. 8B) the successful silencing of luciferase in LM2-4luc+/H2N upon treatment with 30 or 60 nM siLUC (siRNA against luciferase) delivered by spike protein-conjugated NP (spike-NP) at 2 wt. % of siRNA. siSCR=scrambled siRNA control.

FIGS. 9A and 9B show (FIG. 9A) luciferase gene knock down with luciferase siRNA delivered with CaP-L as in Table 5 to H2N (breast) cell line. FIG. 9B shows that the treatment was not toxic to cells as indicated by unchanged total protein level of the treated cells compared to untreated cells. SiRNA dose was 50 nM; protein analysis was two days post treatment.

FIG. 10 shows populations of activated dendritic cells (MHCII+ CD80+ CD11c+ cells) after treatment with CpG, siSTAT3-NP, CpG-NP, siSTAT3-CpG-NP (AIRISE-02), or AIRISE-CoV (an immunogenic construct containing spike protein, siSTAT3, and CpG). Different treatments were administered to mice by footpad injection. Dose was 0.5 mg NP (2 wt. % siSTAT3, 4 wt. % CpG, and/or 3 wt. % SARS-CoV-2 spike protein antigen). Two days after treatment, draining lymph nodes (DLN) and non-draining lymph nodes (NDLN) were collected and processed into single cells for flow cytometry analysis. *p<0.05 vs saline (n=3 per group).

FIG. 11 shows humoral responses to SARS-CoV-2 spike (S) antigen in BALB/c mice vaccinated with AIRISE-CoV. 8-wk-old BALB/c mice were vaccinated (Dose 1: day 0; dose 2: day 17) by footpad injection of 80 μl AIRISE-CoV (0.5 mg NP, 2 wt. % siSTAT3, 4 wt. % CpG, 3 wt. % SARS-CoV-2 spike protein antigen). Serum was collected on day 16 (dose 1 effect) and 38 (dose 2 effect) to assess the level of SARS-CoV-2 S IgG antibodies by ELISA after serial dilution of the serum. Data represent mean OD₄₅₀ nm values (Mean±SD) for n=2 (untreated) and n=3 (AIRISE-CoV).

FIGS. 12A and 12B show humoral responses to SARS-CoV-2 spike (S) antigen in BALB/c mice vaccinated with AIRISE-CoV after 12 weeks. 8-wk-old BALB/c mice (M1-M3 represent mouse 1, 2, and 3) were vaccinated (dose 1: day 0; dose 2: day 17) by footpad injection of (FIG. 12A) 80 μl AIRISE-CoV (0.5 mg NP, 2 wt. % siSTAT3, 4 wt. % CpG, 3 wt. % SARS-CoV-2 spike protein antigen) or (FIG. 12B) 80 μl of AIRISE-CoV utilizing 2 SARS-CoV-2 spike peptides as antigen (0.5 mg NP, 2 wt. % siSTAT3, 4 wt. % CpG, 3 wt. % SARS-CoV-2 spike peptide antigen). Serum was collected on day 80 to assess the level of SARS-CoV-2 S IgG antibodies by ELISA. Data represent mean OD₄₅₀ nm values (Mean±SD) of 2 experimental replicates for each immunized or naïve mouse.

FIG. 13 shows humoral responses to SARS-CoV-2 spike (S) antigen in BALB/c mice vaccinated with two doses of AIRISE-CoV. 8-wk-old BALB/c mice were vaccinated (dose 1: day 0; dose 2: day 17) by footpad (f.p.) injection of AIRISE-CoV (0.5 mg NP, 2 wt. % siSTAT3, 4 wt. % CpG, 3 wt. % SARS-CoV-2 spike protein antigen). Serum was collected on different weeks (weeks 3-54) post vaccination to assess the level of SARS-CoV-2 S IgG antibodies by ELISA. Data represent mean OD₄₅₀ nm values (Mean±SD) of 5 immunized mice and one naïve mouse.

FIGS. 14A-C shows humoral responses to SARS-CoV-2 spike (S) antigen in BALB/c mice vaccinated with a single dose of AIRISE-CoV, siSTAT3-Spike-NP, or CpG-Spike-NP, respectively. 8-wk-old BALB/c mice were vaccinated by intramuscular (i.m.) injection with a single dose of (A) AIRISE-CoV (0.4 mg NP, 2 wt. % siSTAT3, 4 wt. % CpG, 3 wt. % SARS-CoV-2 spike protein antigen; n=4), (B) siSTAT3-Spike-NP (0.4 mg NP, 2 wt. % siSTAT3, 3 wt. % spike protein antigen; n=3), or (C) CpG-Spike-NP (0.4 mg NP, 4 wt. % CpG, 3 wt. % spike protein antigen; n=3). Serum was collected on different weeks (weeks 6-36) post vaccination to assess the level of SARS-CoV-2 S IgG antibodies by ELISA. Data represent mean OD₄₅₀ nm values (Mean±SD) of 3-4 immunized mice and one naïve mouse.

FIG. 15 shows inhibition of SARS-CoV-2 pseudo-virus infection of HEK293-hACE2 cells by immunized sera (from FIG. 11). Graphs showing % GFP+ cells under different dilutions of sera from mice immunized with AIRISE-CoV vs. naïve mice. Calculated neutralizing titers (dilution required to neutralize 50% of virus; NTH′) values are presented in Table 6.

FIGS. 16A and 16B are graphs illustrating cell viability of BMDC (FIG. 16A) and J774 (FIG. 16B) after treatment with siSTAT3-NP or siSTAT3-CpG-NP. NP dose is 35 μg/ml (2 wt. % siRNA; 7 wt. % CpG), 2 days post-treatment.

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled “2LW1153.txt (Sequence Listing.txt)” created on or about Jul. 12, 2021, with a file size of 16 KB, contains the sequence listing for this application and is hereby incorporated by reference in its entirety.

SEQ ID NOs: 1-8 are the amino acid sequences of representative SARS-CoV-2 T-cell and/or B-cell epitopes, from spike protein (SEQ ID NOs: 1-5), nucleocapsid protein (SEQ ID NO: 6), membrane protein (SEQ ID NO: 7), and envelope protein (SEQ ID NO: 8).

SEQ ID NOs: 8-89 and 91 are the nucleic acid sequences corresponding to exemplary siRNAs, as described in Table 2 (below). Though not included in each sequence in the Sequence Listing, each siRNA may optionally include a deoxythymidine dinucleotide (dTdT) or other deoxy-dinucleotide (e.g., dTdG) overhang at the 3′ end.

SEQ ID NO: 90 is an ovalbumin peptide used to stimulate T cells.

### DETAILED DESCRIPTION

Described herein are immunogenic constructs for inducing an immune response, e.g., for treatment or prevention, to an infectious agent, e.g., a virus, such as a beta-coronavirus infection such as a SARS-CoV-2 infection, a SARS-CoV-1 infection, a MERS-CoV infection, or other viruses and pathogens. These novel immunogenic constructs for infectious diseases utilize a single delivery carrier to deliver antigen, adjuvant, and, in some embodiments, siRNA to modulate immunosuppressive genes to stimulate immunity.

The immunogenic construct contains a nanoparticle (e.g., a mesoporous silica nanoparticle (MSNP)), a cationic polymer (e.g., PEI), a stabilizer (e.g., PEG), and an antigen, and, in some embodiments, at least one adjuvant (e.g., CpG) and/or oligonucleotide (e.g., siRNA). Combinations of various additional agents are also contemplated. Immunogenic constructs of the disclosure may also include more than one type of cationic polymer, stabilizer, antigen, adjuvant, and/or oligonucleotide. For example, immunogenic constructs may include multiple, different oligonucleotides and/or antigens that act on the same or different target infectious agent(s). The use of such additional agents may provide an additive or synergistic effect.

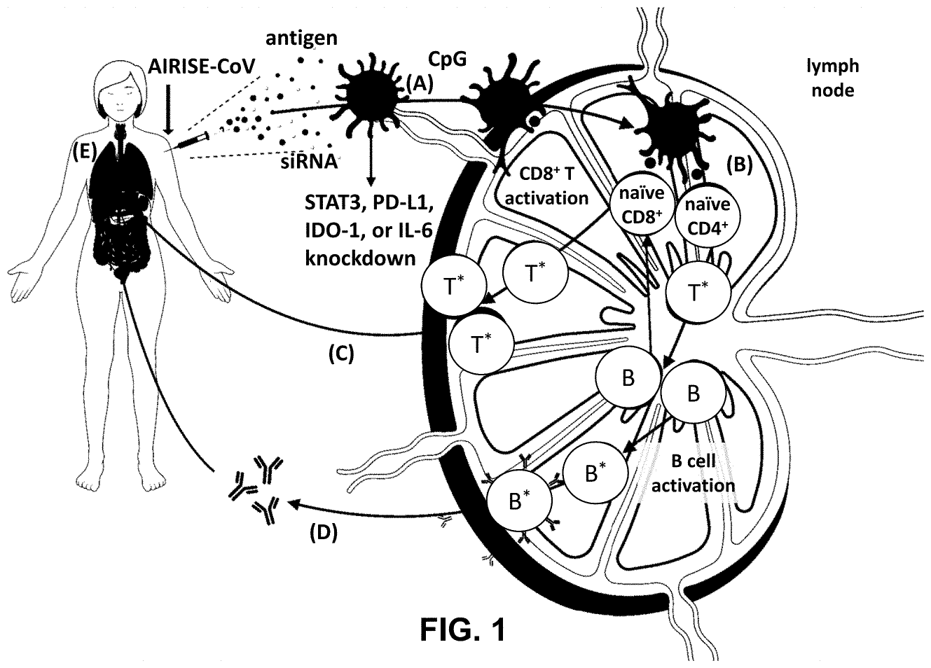

The immunogenic constructs of the disclosure can be used to co-deliver adjuvants (e.g., CpG oligonucleotides), viral antigens (e.g., proteins or peptides) or antigen producing agents (e.g., mRNA or pDNA), and optionally siRNAs, to induce potent long-lasting immunity to novel infectious diseases (FIG. 1). The immunogenic constructs prime the body's antigen-presenting cells (e.g., dendritic cells, B cells, and macrophages) to utilize the antigens to activate effector and memory T lymphocytes and humoral immune response that recognize infectious agent proteins. Such immunogenic constructs may prevent future infection or reduce disease severity.

Aspects of the current disclosure are now described with additional details and options as follows: (I) Definitions; (II) Nanoparticles; (III) Cationic Polymers; (IV) Stabilizers; (V) Adjuvants; (VI) Antigens and Antigen Producing Agents; (VII) Oligonucleotides; (VIII) Targeting Agents; (IX) Labeling Agents; (X) Immunogenic Construct Synthesis; (XI) Immunogenic Constructs including Lipid-Coated Nanoparticles; (XII) Immunogenic Construct Formulations and Methods of Use; (XIII) Exemplary Embodiments; (XIV) Experimental Examples (including Examples 1-8); and (XV) Closing Paragraphs. These headings do not limit the interpretation of the disclosure and are provided for organizational purposes only.

### I. Definitions

To facilitate the understanding of this disclosure, a number of terms are defined below. Terms defined herein have meanings as commonly understood by a person of ordinary skill in the areas relevant to the disclosure. The terminology herein is used to describe specific embodiments of the disclosure, but their usage does not limit the disclosure, except as outlined in the claims.

The term “CpG motif” refers to a 5′ C nucleotide connected to a 3′ G nucleotide through a phosphodiester internucleotide linkage or a phosphodiester derivative internucleotide linkage. In some embodiments, a CpG motif includes a phosphodiester internucleotide linkage. In some embodiments, a CpG motif includes a phosphodiester derivative internucleotide linkage.

As used herein, the term “Class A CpG ODN” (also referred to as “A-class CpG ODN”, “D-type CpG ODN”, or “Class A CpG DNA sequence”) is used in accordance with its common meaning in the biological and chemical sciences and refers to a CpG motif including oligodeoxynucleotide including one or more of poly-G sequence at the 5′, 3′, or both ends; an internal palindrome sequence including CpG motif; or one or more phosphodiester derivatives linking deoxynucleotides. In some embodiments, a Class A CpG ODN includes poly-G sequence at the 5′, 3′, or both ends; an internal palindrome sequence including CpG motif; and one or more phosphodiester derivatives linking deoxynucleotides. In some embodiments, the phosphodiester derivative is phosphorothioate. Examples of Class A CpG ODNs include ODN D19, ODN 1585, ODN 2216, and ODN 2336.

The terms “Class B CpG ODN” or “B-class CpG ODN” or “K-type CpG ODN” or “Class B CpG DNA sequence” are used in accordance with their common meaning in the biological and chemical sciences, and refer to a CpG motif including oligodeoxynucleotide including one or more of a 6mer motif including a CpG motif; phosphodiester derivatives linking all deoxynucleotides. In some embodiments, a Class B CpG ODN includes one or more copies of a 6mer motif including a CpG motif and phosphodiester derivatives linking all deoxynucleotides. In some embodiments, the phosphodiester derivative is phosphorothioate. In some embodiments, a Class B CpG ODN includes one 6mer motif including a CpG motif. In some embodiments, a Class B CpG ODN includes two copies of a 6mer motif including a CpG motif. In some embodiments, a Class B CpG ODN includes three copies of a 6mer motif including a CpG motif. In some embodiments, a Class B CpG ODN includes four copies of a 6mer motif including a CpG motif. Examples of Class B CpG ODNs include ODN 1668, ODN 1826, ODN 2006, and ODN 2007.

The terms “Class C CpG ODN” or “C-class CpG ODN” or “C-type CpG DNA sequence” are used in accordance with their common meaning in the biological and chemical sciences and refers to an oligodeoxynucleotide including a palindrome sequence including a CpG motif and phosphodiester derivatives (phosphorothioate) linking all deoxynucleotides. Examples of Class C CpG ODNs include ODN 2395 and ODN M362.

As used herein, “immunogenic” refers to the ability of an agent (e.g., an immunogenic construct, a component thereof, or a composition containing an immunogenic construct), to trigger an immune response, e.g., as measured by in vitro assays (e.g. mixed lymphocyte reaction; cytotoxic T cell killing, upregulation of cytokines upon stimulation of immune cells with antigen, etc.), ex vivo assays (e.g. antibody neutralizing titers via microneutralization assay; antigen-specific antibodies and antibody-secreting B cells via ELISA assays) and in vivo assays that confirm successful induction of cellular and humoral immunity (e.g., ability of immunization to protect a live specimen from viral challenge).

As used herein, the term “immunogenic amount” refers to an amount of an immunogenic construct or composition that induces an immune response in a subject (e.g., reflected by an increase in antibody titer in the subject as determined by conventional techniques, such as ELISA).

The term “infectious agent,” as used herein, refers to agents that cause an infection and/or a disease. Infectious agents include viruses, bacteria, fungi, and parasites, or a combination thereof. In some embodiments, the infectious agent is a virus. Additional infectious agents are discussed herein, and/or will be known to those of ordinary skill in the art. In instances, the infectious agent may be referred to as a “target” of an immunogenic construct as described herein. For instance, a viral target may be a coronavirus, a Corynebacterium, an ebolavirus, an orthomyxovirus, a hepatovirus, a Haemophilus bacterium, HIV, HPV, a morbillivirus, a Mycobacterium, a meningococcus bacterium, an orthorubulavirus, a norovirus, a Streptococcus, an enterovirus, an orthopneumovirus, a rotavirus, a rubivirus, a herpesvirus, a Clostridium bacterium, a bordatella bacterium, or a flavivirus. Pathogens are also referred to as infectious agents.

The term “infectious disease,” as used herein, refers to diseases caused by infectious agents such as bacteria, viruses, parasites, or fungi. In some embodiments, the infectious disease is a viral infection. Examples of the infectious diseases include coronavirus-based infections (such as middle east respiratory syndrome (MERS), severe acute respiratory syndrome (SARS), and coronavirus diseases (e.g., COVID-19)); Corynebacterium-based infections (such as diphtheria); ebolavirus-based infections (such as Ebola); orthomyxoviridae virus-based infections (such as influenza A, B, or C); hepatovirus A, B, C, D, or E-based infections (such as hepatitis); Haemophilus-based infections (such as hib disease); human immunodeficiency virus (HIV)-based infections (such as acquired immunodeficiency syndrome (AIDS)); human papillomavirus (HPV)-based infections; Morbillivirus-based infections (such as measles); Mycobacterium-based infections (such as tuberculosis); Neisseria-based infections (such as meningitis); Orthorubulavirus-based infections (such as mumps); norovirus-based infections; Streptococcus-based infections; enterovirus-based infections (such as polio); Orthopneumovirus-based infections; rotavirus-based infections; Rubivirus-based infections (such as rubella); herpesvirus-based infections (such as herpes, chickenpox, and shingles); Clostridium-based infections (such as tetanus and botulism); Bordatella-based infections (such as pertussis); Flavivirus-based infections (such as Zika); and so on. Additional infectious diseases are discussed herein, and/or will be known to those of ordinary skill in the art (e.g., Pati et al., Front Immunol. 9:2224, 2018 (16 pages), and references cited therein).

The terms “biologically acceptable excipient” and “pharmaceutically acceptable excipient,” as used herein, refer to any inactive ingredient (for example, a vehicle capable of suspending an immunogenic construct) having the properties of being nontoxic and non-inflammatory in a subject. Typical excipients include, for example: carriers, binders, fillers, lubricants, emulsifiers, suspending agents, sweeteners, flavorings, preservatives, buffers, wetting agents, disintegrants, effervescent agents and other conventional excipients and additives and/or other additives that may enhance stability, delivery, absorption, half-life, efficacy, pharmacokinetics, and/or pharmacodynamics, reduce adverse side effects, or provide other advantages for biological and/or pharmaceutical and/or dietary supplement use. In some embodiments, the acceptable excipient includes an adjuvant that is not bound to the immunogenic construct.

As used herein, “pDNA” refers to plasmid DNA, for instance a plasmid encoding at least one antigen of an infectious agent.

As used herein, the term “preventing” means decreasing the risk of (e.g., by 1%, 2%, 3%, 4%, 5%, 6%, 7%, 8%, 9%, 10%, 20%, 30%, 40%, 50%, 60%, 70%, 80%, 90%, 95%, 97%, 99%, or about 100%) contracting an infectious disease, e.g., a viral infection (such as an infection by a beta-coronavirus such as SARS-CoV-2, SARS-CoV-1, MERS-CoV, or a related virus), a bacterial infection, a fungal infection, or a parasitic infection. To determine whether the prevention is effective, a comparison can be made between the subject who received a composition of the disclosure and a similarly-situated subject (e.g., one at risk of a viral infection, such as a SARS-CoV-2, SARS-CoV-1, or MERS-CoV infection, or an infection by a related virus) who did not receive the composition. A comparison can also be made between the subject who received the composition and a control, a baseline, or a known level of measurement.

The term “subject,” as used herein, can be a human, a non-human primate, or a non-primate mammal, such as a dog, a cat, a horse, a cow, a pig, a horse, a goat, a monkey, a rat, a mouse, and/or a sheep. In some embodiments, the subject is a human.

As used herein, the term “TLR-binding DNA substituent” refers to a substituent or moiety capable of binding to a toll-like receptor (“TLR”), including at least one deoxyribonucleic acid. In some embodiments, the TLR-binding DNA substituent is a nucleic acid. In some embodiments, the TLR-binding DNA substituent includes at least one nucleic acid analog. In some embodiments, the TLR-binding DNA substituent includes at least one nucleic acid analog having an alternate backbone (e.g., phosphodiester derivative (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite), peptide nucleic acid backbone(s), LNA, or linkages). In some embodiments, a TLR-binding DNA substituent includes DNA. In some embodiments, all nucleotide in a TLR-binding DNA substituent are deoxyribonucleotides. In some embodiments, a TLR-binding DNA substituent includes or is DNA having internucleotide linkages selected from phosphodiesters and phosphodiester derivatives (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite, or combinations thereof). In some embodiments, a TLR-binding DNA substituent includes DNA having internucleotide linkages selected from phosphodiesters and phosphorothioates. In some embodiments, a TLR-binding DNA substituent includes or is DNA having backbone linkages selected from phosphodiesters, phosphorothioates, and phosphorodithioates. In some embodiments, a TLR-binding DNA substituent includes or is DNA including phosphodiester backbone linkages. In some embodiments, a TLR-binding DNA substituent includes or is DNA including phosphorothioate backbone linkages. In some embodiments, a TLR-binding DNA substituent includes or is DNA including phosphorodithioate backbone linkages. In some embodiments, a TLR-binding DNA substituent preferentially binds TLR9 over other TLRs. In some embodiments, a TLR-binding DNA substituent specifically binds TLR9. In some embodiments, a TLR-binding DNA substituent specifically binds TLR3. In some embodiments, a TLR-binding DNA substituent specifically binds TLR7. In some embodiments, a TLR-binding DNA substituent specifically binds TLR8. In some embodiments, a TLR-binding DNA substituent specifically binds a cellular sub-compartment (e.g., endosome) associated TLR (e.g., TLR3, TLR7, TLR8, or TLR9). In some embodiments, a TLR-binding DNA substituent includes or is a G-rich oligonucleotide. In some embodiments, a TLR-binding DNA substituent includes a CpG motif (i.e., is a CpG oligodeoxynucleotide (ODN)). In some embodiments, the CpG motif is unmethylated. In some embodiments, a TLR-binding DNA substituent is a Class A CpG oligodeoxynucleotide (ODN). In some embodiments, a TLR-binding DNA substituent is a Class B CpG oligodeoxynucleotide (ODN). In some embodiments, a TLR-binding DNA substituent is a Class C CpG oligodeoxynucleotide (ODN). In some embodiments, a TLR-binding DNA substituent (e.g., TLR9-binding DNA substituent) includes deoxyribonucleic acids with A, G, C, or T bases and phosphodiester linkages and/or phosphodiester derivative linkages (e.g., phosphorothioate linkage(s)).

As used herein, the term “treatment” or “treating” refers to reducing, decreasing, decreasing the progression of, or decreasing the side effects of (e.g., by 1%, 2%, 3%, 4%, 5%, 6%, 7%, 8%, 9%, 10%, 20%, 30%, 40%, 50%, 60%, 70%, 80%, 90%, 95%, 97%, 99%, or about 100%) an infectious disease, e.g., viral infection, e.g., a beta-coronavirus (e.g., SARS-CoV-2, SARS-CoV-1, or MERS-CoV infection, or a related virus) infection. To determine whether the treatment is effective, a comparison can be made between the treated subject and a similarly-situated subject (e.g., one with, or at risk of a viral infection, such as a SARS-CoV-2, SARS-CoV-1, or MERS-CoV infection, or an infection by a related virus infection) who did not receive treatment. A comparison can also be made between the treated subject and a control, a baseline, or a known level or measurement. Treating a viral infection (e.g., a beta-coronavirus infection such as a SARS-CoV-2, SARS-CoV-1, or MERS-CoV infection, or an infection by a related virus), includes one or more of reducing viral load (e.g., by 1%, 2%, 3%, 4%, 5%, 6%, 7%, 8%, 9%, 10%, 20%, 30%, 40%, 50%, 60%, 70%, 80%, 90%, 95%, 97%, 99%, or about 100%), reducing the number of days of hospitalization of the subjection (e.g., by 1, 2, 3, 4, 5, 6, 7, 8, 9, 10, 20, 30, 40, 50, or more days), reducing the number of days the subject requires antiviral therapy (e.g., by 1, 2, 3, 4, 5, 6, 7, 8, 9, 10, 20, 30, 40, 50, or more days), and/or reducing the dose of antiviral therapy the subject needs.

As used herein, the term “vaccine” refers to an agent (e.g., an immunogenic construct, a component thereof, or a composition containing an immunogenic construct) capable of inducing an immune response against an infectious agent in a subject and/or treating and/preventing an infection and/or a disease associated with the infectious agent.

### II. Nanoparticles

Nanoparticles useful with the compositions and methods of the disclosure include, without limitation, mesoporous silica nanoparticles (e.g., MSNPs), iron oxide nanoparticles, silver nanoparticles, gold nanoparticles, calcium phosphate, inorganic nanoparticles, carbon nanotubes, liposomes, lipid nanoparticles, or cationic polymeric particles. Nanoparticles may or may not be porous. Exemplary sizes for the nanoparticle cores are from about 5 nm to about 999 nm, about 5 nm to about 90 nm, about 5 nm to about 20 nm, about 20 nm to about 400 nm, about 20 nm to about 500 nm, about 20 nm to about 100 nm, about 20 nm to 200 nm, about 30 nm to about 100 nm, about 30 nm to about 80 nm, about 30 nm to about 60 nm, about 40 nm to about 80 nm, about 50 nm to 400 nm, about 50 to 500 nm, about 70 nm to about 90 nm, about 100 nm to about 200 nm, about 200 nm to about 500 nm, about 500 nm to about 999 nm, or about 5 nm, about 10 nm, about 20 nm, about 30 nm, about 40 nm, about 50 nm, about 60 nm, about 70 nm, about 80 nm, about 90 nm, or about 100 nm. Generally, the nanoparticle cores are spherical, although other shapes, such as rods and discs, may also be used. In some embodiments, the nanoparticle is a mesoporous silica nanoparticle (MSNP).

In some embodiments, the nanoparticle has adjuvant or immunostimulatory properties. Exemplary nanoparticles having adjuvant or immunostimulatory properties include liposomes, lipoplexes, lipid-based particles, polyplexes, polymeric particles, inorganic particles (e.g., aluminum salt particles and calcium phosphate nanoparticles), virus-like particles, or nanoparticles formed from one or more of 1,2-dioleoyl-3-trimethylammonium propane (DOTAP), cholesterol, 3β-[N—(N′,N′-dimethylaminoethane)-carbamoyl]cholesterol, phosphatidylcholine/cholesterol, chitosan, poly-γ-glutamic acid (γ-PGA), hyaluronic acid, polyethylenimine (PEI), poly(propylacrylic acid), polypropylene sulfide (PPS), poly(lactic-co-glycolic acid) (PLGA), amylopectin, maltodextrin, polystyrene, gold, cobalt oxide, alum, tri-palmitoyl-S-glyceryl cysteine (PAM3Cys), squalene, Montanide ISA 50V, Montanide ISA 51, Montanide ISA 201, Montanide ISA 206, and Montanide ISA 720.

To make a nanoparticle platform (NP), additional components are attached to nanoparticles by various mechanisms, covalently or noncovalently. For example, cationic polymers may be attached to nanoparticles by charge, e.g., for silica or iron oxide nanoparticles. Alternatively, the surfaces of the nanoparticles may be altered to include reactive moieties for conjugation to cationic polymers and/or other components, or the cationic polymers or other components may include a moiety that binds to the nanoparticles. For example, nanoparticle cores such as silica, silicon, gold, iron oxide, and silver nanoparticles, as well as carbon nanotubes, may be modified with reactive moieties such as thiols, phosphonate, carboxylate, and amines prior to attachment with cationic polymers and other components. Cationic polymers and other components may be modified to include these or other moieties, including maleimide, N-hydroxy succinimidyl (NHS) esters, or azides, prior to binding to the nanoparticle cores. Components may be attached directly to nanoparticles, either on the surface or within pores (if present). Large molecule cargos such as proteins, mRNA, or plasmid DNA (pDNA) are attached on an external surface of the nanoparticles (or optionally, on a coating applied to a nanoparticle core—that is, loaded onto a NP), while smaller molecules such as dyes can be attached inside pores of the nanoparticles or NP as well as on the exterior surface. The immunogenic constructs of the present disclosure advantageously maintain their sub-micron sizes after additional components are loaded.

### III. Cationic Polymers

In some embodiments, nanoparticles, such as MSNPs, are coated with cationic polymers or other compounds. The cationic polymer may bind to the surface of the nanoparticle using any appropriate means. In some embodiments, the cationic polymer binds to the nanoparticle via electrostatic interaction. The cationic polymer may be any polymer with a positive charge, such as, but not limited to, PEI, polyamidoamine, poly(allylamine), poly(diallyldimethylammonium chloride), chitosan, poly(N-isopropyl acrylamide-co-acrylamide), poly(N-isopropyl acrylamide-co-acrylic acid), poly(L-lysine), diethylaminoethyl-dextran, poly-(N-ethyl-vinylpyridinium bromide), poly(dimethylamino)ethyl methacrylate), or poly(ethylene glycol)-co-poly(trimethylamine-ethylmethacrylate chloride). Other cationic polymers will be apparent to those of skill in the art, and may be found, for example, in Polymer Handbook, 4th Edition, Edited by: Brandrup, E. H. Immergut, and E. A. Grukle; John Wiley & Sons, 2003).

The cationic polymers may be linear or branched. In some embodiments, the cationic polymers may range in size from about 500 Da to 25 kDa and may be branched or linear. For example, branched PEI with an average size of 1.8 kDa to 10 kDa may be loaded onto the nanoparticle (yielding a nanoparticle platform; NP). The ratio of cationic polymer to nanoparticle may be varied depending on the desired result. The cationic polymer may be present at 1 to 50 wt. % of the NP, e.g., 5 to 40 wt. %, 10 to 30 wt. %, 20 to 30 wt. %, 5 to 15 wt. %, 5 to 20 wt. %, 5 to 25 wt. %, 5 to 30 wt. %, 10 to 20 wt. %, 10 to 25 wt. %, or 25 to 40 wt. %, e.g., about 5, 10, 15, 20, 25, 30, or 35 wt. %. In some embodiments, the cationic polymer is present at 10 to 20 wt. % of NP.

In some embodiments, the cationic polymer is crosslinked, e.g., with a cleavable disulfide bond, pre- or post-coating on the nanoparticle. In some embodiments, the attached cationic polymer is crosslinked after binding to the nanoparticles, e.g., MSNP, using, for example, DSP (dithiobis[succinimidyl propionate]), DTSSP (3,3′-dithiobis(sulfosuccinimidyl propionate), and DTBP (dimethyl 3,3′-dithiobispropionimidate). The crosslinking may occur in the absence or presence of free cationic polymer in solution. In other embodiments, the cationic polymer is not crosslinked.

### IV. Stabilizers

A stabilizer may be conjugated to the nanoparticle and/or the cationic polymer, e.g., by any appropriate means. In some embodiments, a stabilizer is conjugated to an amine or other reactive group of a cross-linked cationic polymer coated on the nanoparticle (e.g., a MSNP). Exemplary stabilizers include PEG, dextran, polysialic acid, hyaluronic acid, polyvinyl pyrrolidone, polyvinyl alcohol, and polyacrylamide, or a combination thereof.

A stabilizer may have multiple chemically reactive groups, e.g., for attachment to the nanoparticle, cationic polymer, and/or other component. For example, a reactive stabilizer, e.g., a PEG derivative, may have two functional moieties, such as maleimide-PEG-N-hydroxysuccinimidyl ester (Mal-PEG-NHS), which contains both a maleimide and an activated ester on each end. The stabilizer, e.g., PEG, used in conjunction with the compositions and methods of the disclosure generally has a molecular weight ranging between 500 Da-40 kDa, e.g., 2-10 kDa. The stabilizer may be present at 1 to 50 wt. % of the NP, e.g., 5 to 30 wt. %, 10 to 20 wt. %, 10 to 25 wt. %, 5 to 15 wt. %, 5 to 20 wt. %, 5 to 25 wt. %, or 1 to 10 wt. %, e.g., about 5, 10, 15, 20, 25, 35, 40 or 45 wt. %.

In some embodiments, the stabilizer (e.g., PEG) is introduced to enhance NP stability (e.g., reduce aggregation and precipitation) and/or to protect cargo molecule(s), such as siRNA, miRNA, mRNA, and pDNA, e.g., from enzymatic degradation.

The stabilizer (e.g., PEG) used in conjunction with the compositions and methods of the disclosure generally has a molecular weight ranging between 500 Da-40 kDa, e.g., 5 kDa, 2-5 kDa, 2-10 kDa, 5-10 kDa. The stabilizer in various embodiments is present at 1 to 50 wt. % of the nanoparticle platform (NP), e.g., 5 to 30 wt. %, 10 to 20 wt. %, 10 to 25 wt. %, 5 to 15 wt. %, 5 to 20 wt. %, 5 to 25 wt. %, or 1 to 10 wt. %, e.g., about 5, 10, 15, 20, 25, 35, 40 or 45 wt. %. The size and the density of the stabilizers can be optimized to accommodate large cargos such as proteins and mRNA

In some embodiments, the stabilizer is introduced before cargo loading. In some embodiments, the stabilizer is introduced after cargo loading. In some embodiments, stabilizer is introduced both before and after cargo loading, and/or concurrently with the loading of at least one cargo molecule.

Some nanoparticles (such as mesoporous silica and iron oxide nanoparticles coated with PEI and PEG) can protect antigens and antigen producing agents for long term storage and during transportation such that storing the material at very low temperature (e.g., −80° C.) is not required.

### V. Adjuvants

The constructs provided herein may include at least one adjuvant. The adjuvant may be contained within the nanoparticle or otherwise associated with the nanoparticle, the cationic polymer, or the stabilizer via non-covalent or covalent interactions, including hydrogen bonding, Van der Waals interactions, electrostatic interactions, hydrophobic interactions, and chemical conjugation with moieties on the nanoparticle. Chemical conjugations include thiol-maleimide, NHS ester-amine, azide-alkyne, and other click chemistries. In some embodiments, the adjuvant is thiolated and is conjugated to a stabilizer containing maleimide groups via a thiol-maleimide reaction (see International Application No. PCT/US2016/022655, which is incorporated by reference herein in its entirety). In some embodiments, the adjuvant is loaded electrostatically on the cationic polymer coated on the nanoparticle. The adjuvant may be present at 1-20 wt. % of the NP, e.g., 1-10 wt. %, 2-7 wt. %, 2-4 wt. %, 2-10 wt. %, 5-10 wt. %, 10-20 wt. %., e.g., about 4 wt. %, 5 wt. %, 6 wt. %, 7 wt. %, 10 wt. %, or 20 wt. %.

Adjuvants may also be part of or conjugated with therapeutic agents, e.g., an oligonucleotide, such as a siRNA, that knocks down a target gene, can be designed to contain an immune-stimulatory sequence.

Generally, an adjuvant is any substance whose admixture into a vaccine composition increases or otherwise modifies the immune response to the antigen. The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased antigen-specific T cell proliferation, death of target cells, or cytokine secretion. An adjuvant may also alter an immune response, for example, by changing a primarily humoral or Th2 response into a primarily cellular, or Th1 response.

Suitable adjuvants include TLR-binding DNA substituents such as CpG oligonucleotides (e.g., ISS 1018; Amplivax; CpG ODN 7909, CpG ODN 1826, CpG ODN D19, CpG ODN 1585, CpG ODN 2216, CpG ODN 2336, ODN 1668, ODN 1826, ODN 2006, ODN 2007, ODN 2395, ODN M362, and SD-101), DNA TLR agonists that contain a CpG sequence (e.g., dSLIM), non-CpG DNA TLR agonists (e.g., EnanDIM), and cationic peptide-conjugated CpG oligonucleotides (e.g., IC30, IC31); RNA TLR agonists (e.g., Poly I:C and Poly-ICLC); aluminum salts (e.g., aluminum hydroxide, aluminum phosphate, aluminum chloride, and aluminum potassium sulfate); anti-CD40 antibodies (e.g., CP-870,893); cytokines, such as granulocyte-macrophage colony-stimulating factor (GM-CSF); small molecule TLR agonists (e.g., imiquimod, resiquimod, gardiquimod, and 3M-052); fusion proteins (e.g., ImuFact IMP321, CyaA, and ONTAK); oil- or surfactant-based adjuvants such as MF59, Montanide IMS 1312, Montanide ISA 206, Montanide ISA 50V, and Montanide ISA-51; a plant extract such as QS21 stimulon (Aquila Biotech, Worcester, Mass., USA), which is derived from saponin; mycobacterial extracts and synthetic bacterial cell wall mimics, such as lipopolysaccharides (e.g., monophosphoryl lipid A, OM-174, OM-197-MP-EC, and Pam3Cys); xanthenone derivatives (e.g., Vadmiezanvadimezan); mixtures thereof (e.g., AS-15); and other proprietary adjuvants such as Ribi's Detox, Quil, or Superfos. Several immunological adjuvants (e.g., MF59 specific for dendritic cells and their preparation have been described previously (Dupuis et al., Cell Immunol. 186(1): 18-27, 1998; Allison, Dev Biol Stand.; 92:3-11, 1998). Cytokines may also be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589) and acting as immunoadjuvants (e.g., IL-12) (Gabrilovich et al., J Immunother Emphasis Tumor Immunol. (6):414-418, 1996). Toll-like receptors (TLRs) or agents that activate TLRs may also be used as adjuvants, and are important members of the family of pattern recognition receptors (PRRs) which recognize conserved motifs shared by many micro-organisms, termed “pathogen-associated molecular patterns” (PAMPS).

In some embodiments, the adjuvant includes a CpG oligonucleotide. CpG immuno-stimulatory oligonucleotides have also been reported to enhance the effects of adjuvants in a vaccine setting. Without being bound by any particularly mechanistic theory, CpG oligonucleotides act at least in part by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of TH1 cells and strong cytotoxic T-lymphocyte (CTL) generation, even in the absence of CD4 helper T-cells. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enabled the antigen doses to be reduced by two orders of magnitude, with comparable antibody responses to the full-dose vaccine without CpG in some experiments (Krieg, Nature Reviews, Drug Discovery, 5:471-484, 2006). U.S. Pat. No. 6,406,705 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY). Other TLR binding molecules such as RNA binding TLR 7, TLR 8 and/or TLR 9 may also be used.

Xanthenone derivatives such as, for example, vadimezan or AsA404 (also known as 5,6-dimethylaxanthenone-4-acetic acid (DMXAA)), may also be used as adjuvants according to embodiments of the disclosure. Alternatively, such derivatives may also be administered in parallel to the vaccine of the disclosure, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al., J Immunology, 190:5216-5225, 2013; and Kim et al., ACS Chem Biol, 8:1396-1401, 2013). Other examples of useful adjuvants include chemically modified CpGs (e.g., CpR, Idera), PolyI:C (e.g. polyI:Cl2U), non-CpG bacterial DNA or RNA as well as immunoactive small molecules and antibodies such as cyclophosphamide, sunitinib, bevacizumab, Celebrex™, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present disclosure can readily be determined by the skilled artisan without undue experimentation. Additional adjuvants include colony-stimulating factors, such as Granulocyte Macrophage Colony Stimulating Factor (GM-CSF, sargramostim).

In some embodiments, the adjuvant includes poly-ICLC. Poly-ICLC is a synthetically prepared double-stranded RNA including polyI and polyC strands of average length of 5000 nucleotides, which has been stabilized to thermal denaturation and hydrolysis by serum nucleases by the addition of poly-lysine and carboxymethylcellulose. The compound activates TLR3 and the RNA helicase-domain of MDAS, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′ 5′-OAS and the PI/eIF2a kinase, also known as the PKR (4-6), as well as RIG-I helicase and MDAS.

Examples of immunological adjuvants that can be associated with the immunogenic constructs include TLR ligands, C-Type Lectin Receptor ligands, NOD-Like Receptor ligands, RLR ligands, and RAGE ligands. TLR ligands can include lipopolysaccharide (LPS) and derivatives thereof, as well as lipid A and derivatives thereof including monophosphoryl lipid A (MPL), glycopyranosyl lipid A, PET-lipid A, and 3-O-desacyl-4′-monophosphoryl lipid A. In a specific embodiment, the immunological adjuvant is MPL. In another embodiment, the immunological adjuvant is LPS. TLR ligands can also include TLR3 ligands (e.g., polyinosinic-polycytidylic acid (poly I:C), TLR7 ligands (e.g., imiquimod and resiquimod), and TLR9 ligands.

### VI. Antigens and Antigen Producing Agents

The immunogenic constructs provided herein include at least one antigen or antigen producing agent; exemplar constructs include both at least one antigen and at least one antigen producing agent. In embodiments where the immunogenic construct contains more than one antigen and/or antigen producing agent, these antigens may correspond to/be derived from different infectious agents or they may correspond to/be derived from the same infectious agent. An antigen or antigen producing agent can be considered “of” or “from” an infectious agent when the antigen is capable of eliciting an immune response to the corresponding agent—for instance, when the antigen (or antigen producing agent) is synthetic, engineered, recombinant, and/or produced in a laboratory, or when it is isolated from or extracted from the infectious agent itself.

The antigen or antigen producing agent may be contained partially or fully within the nanoparticle or otherwise associated with the nanoparticle, the cationic polymer, and/or the stabilizer via non-covalent or covalent interactions, including hydrogen bonding, Van der Waals interactions, electrostatic interactions, hydrophobic interactions, and chemical conjugation with moieties on the nanoparticle. Chemical conjugations include thiol-maleimide, NHS ester-amine, azide-alkyne, and other click chemistries. In some embodiments, the antigen or antigen producing agent is thiolated and is conjugated to a stabilizer containing maleimide groups via a thiol-maleimide reaction (see International Application No. PCT/US2016/022655). In some embodiments, the antigen or antigen producing agent is loaded on the cationic polymer via hydrophobic interactions with the nanoparticle. In some embodiments, the antigen or antigen producing agent is electrostatically loaded on the cationic polymer. The antigen or antigen producing agent may be present at 2 wt. %, 3 wt. %, 4 wt. % 5 wt. %, 0.5-20 wt. % of the NP, e.g., 1-15 wt. %, 1.5-10 wt. %, 1-6 wt. %, or 2-5 wt. %.

The antigen is any substance that is recognized as “foreign” by the body and consequently elicits an antigen-specific immune response by the body's immune cells. The antigen is often engulfed by the body's antigen-presenting cells (e.g., dendritic cells) and processed into epitopes that are presented via major histocompatibility complex to T cells and/or B cells to induce antigen-specific immunity. The immune response may be cellular and/or humoral. An increase in cellular immunity is typically manifested by an increase in antigen-specific T-cell activity, proliferation, and enhanced ability of T cells to recognize and eliminate the antigen. An increase in humoral immunity is typically manifested by an increase in antigen-specific B cell activity and proliferation, which produce antibodies capable of recognizing and neutralizing the antigen of interest.

One category of antigen is a recombinant full-length protein or protein subunit that corresponds to a specific protein related to (or derived from) the infectious agent of interest (the target). For example, the antigen may be the full-length SARS-CoV-2 spike glycoprotein, which has been identified as immunogenic (Grifoni et al. Cell Host Microbe. 2020; 27(4): 671-80; Ou et al. Nat Commun. 2020, 11(1): 1620; Walls et al. Cell. 2020; 181(2): 281-92). In addition, the antigen may correspond to SARS-CoV-2 nucleocapsid protein, membrane protein, etc. The antigen may also correspond to a specific functional region of a protein (i.e., protein subunit, or a protein domain). For example, the antigen may correspond to the S1, S2, or RBD region of the SARS-CoV-2 spike glycoprotein.

The antigen(s) may also be a peptide (or several peptides) that correspond to (are derived from) immunogenic sequences in the infectious agent of interest (the target infectious agent). The peptides behave as epitopes that can elicit various immune responses. Antigens may be epitopes selected based on predicted immunogenicity, as analyzed by bioinformatic approaches, and/or experimental data which has implicated them in immune cell stimulation. For example, the peptides may represent positions 494-508 or 1056-1070 of the SARS-CoV-2 spike glycoprotein, which are predicted in both cellular and humoral immunogenicity (Fast et al. bioRxiv. 2020: 2020.02.19.955484).

Regarding antigens made up of several peptides, the antigen(s) may be a cocktail of overlapping (or non-overlapping) peptides that encompass a whole (or nearly the entire) protein, or it may be a mixture of peptides that correspond to immunogenic region(s) of a single protein or two or more different proteins (which may target one or different target infectious organisms). For example, the antigen(s) may be a mix of peptides that includes SARS-CoV-2 spike protein, nucleocapsid protein, and membrane protein. Shown in Table 1 are examples of SARS-CoV-2 T-cell and/or B-cell epitopes that are predicted to be immunogenic based bioinformatic prediction approaches such as Immune Epitope Database and Analysis Resource (IEDB) and Discotope 2.0 prediction algorithm, as well as high sequence similarity to SARS-CoV-1 (e.g. >90%, >80%, >70%, >60%, or >50%), which is the best characterized coronavirus in terms of epitope responses (Grifoni et al. Cell Host Microbe. 2020; 27(4): 671-80; Fast et al. bioRxiv. 2020: 2020.02.19.955484).

TABLE 1

| | | | SEQ |
|---|---|---|---|
| | | | ID |
| Gene | Sequence | Position | NO |
| Spike (S) | KLPDDFTGCV | 424-433 | 1 |
| | SQSIIAYTMSLGAEN | 689-703 | 2 |
| | IPTNFTISVTTEILP | 714-728 | 3 |
| | FGAGAALQIPFAMQM | 888-909 | 4 |
| | AYRFNGIG | | |
| | APHGVVFLHVTYVPA | 1056-1070 | 5 |
| Nucleocapsid | ATKAYNVTQAFGRRG | 267-281 | 6 |
| (N) | | | |
| Membrane (M) | IASFRLFARTRSMWS | 97-111 | 7 |
| Envelope (E) | VKPSFYVYSRVKNLN | 52-66 | 8 |

Other examples for predicted immunogenic epitopes can be found throughout literature (Prachar et al. bioRxiv. 2020: 2020.03.20.000794; Chour et al. medRxiv. 2020; 2020.05.04.20085779) and SARS-CoV-2 antigens' vendors' websites (e.g., Sino Biological, Creative Diagnostics, Sengenics, ABclonal Technology). Prediction tools for identifying immunogenic regions based on MHC binding ability are also widely available.

In various embodiments, the antigen producing agent is a nucleic acid, such as mRNA or pDNA, that encodes a specific protein or peptide corresponding to or specific for the target infectious agent. Once administered to the subject, the mRNA or pDNA enters the cell's cytoplasm where it is expressed (translation for mRNA or transcription/translation for pDNA) into the desired protein that can ultimately activate cellular and humoral immune response. The antigen-encoding sequence can be any sequence that codes for a specific protein or protein subunit; for example, mRNA or pDNA that encodes SARS-CoV-2 spike protein, spike RBD domain, spike 51 domain, etc. To increase potency, stability, and protein yield, the mRNA or pDNA may be subject to codon optimization, use of modified nucleosides, polyadenylation, etc. For example, the design of the 5′ UTR and 3′ UTR are critical for mRNA stability, translation, protein production, and structure; there are several online tools that optimize the design of 5′ UTR and 3′ UTR based on mRNA of interest. For effective antigen expression, the mRNA will be synthesized to comprise the following: 5′ cap-5′ untranslated region (UTR)-antigen-encoding sequence-3′ untranslated region (UTR)-poly A tail. The mRNA may also be non-modified, nucleoside-modified, or self-amplifying. For example, incorporation of modified uridines or modified cytidine may be done to avoid premature recognition by innate immune molecules and improve efficiency of translation.

Suitable additional target antigens are known in the art (e.g., in Pati et al., Front Immunol. 9:2224, 2018 (16 pages), and references cited therein), and are available from commercial government and scientific sources. Additional exemplary antigens are provided below.

Exemplary Viral Antigens: A viral antigen can be isolated from any virus including, but not limited to, a virus from any of the following viral families: Adenoviruses, Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus, including COVID-19), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hantaviridea (e.g., hantavirus), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A and B and C), Papillomaviridea (including both human papillomavirus (HPV) and animal papillomaviruses), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (e.g., rabies virus, measles virus, respiratory syncytial virus, etc.), Rotaviridea (e.g., Rotavirus A-J), Togaviridae (e.g., rubella virus, dengue virus, etc.), Spongiform viruses, and Totiviridae. Suitable viral antigens also include all or part of Dengue protein M or protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomegalovirus, Epstein-Barr, rotavirus, rhinovirus, adenovirus, coxsackieviruses, equine encephalitis, Japanese encephalitis, yellow fever, Rift Valley fever, and lymphocytic choriomeningitis. In additional embodiments, viral antigen markers include peptides expressed by CMV, cold viruses, Epstein-Barr, flu viruses, hepatitis A, B, and C viruses, herpes simplex, HIV, influenza, Japanese encephalitis, measles, polio, rabies, respiratory syncytial, rubella, smallpox, varicella zoster or West Nile virus.

As further particular examples, cytomegaloviral antigens include envelope glycoprotein B and CMV pp65; Epstein-Barr antigens include EBV EBNAI, EBV P18, and EBV P23; hepatitis antigens include the S, M, and L proteins of HBV, the pre-S antigen of HBV, HBCAG DELTA, HBV HBE, hepatitis C viral RNA, HCV NS3 and HCV NS4; herpes simplex viral antigens include immediate early proteins and glycoprotein D; HIV antigens include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include hemagglutinin and neuraminidase; Japanese encephalitis viral antigens include proteins E, M-E, M-E-NS1, NS1, NS1-NS2A and 80% E; measles antigens include the measles virus fusion protein; rabies antigens include rabies glycoprotein and rabies nucleoprotein; respiratory syncytial viral antigens include the RSV fusion protein and the M2 protein; rotaviral antigens include VP7sc; rubella antigens include proteins E1 and E2; and varicella zoster viral antigens include gpI and gpII. Additional particular exemplary viral antigen sequences include: Nef (66-97); Nef (116-145); Gag p17 (17-35); Gag p17-p24 (253-284); and Pol 325-355 (RT 158-188). See Fundamental Virology, Second Edition, eds. Fields, B. N. and Knipe, D. M. (Raven Press, New York, 1991) for additional examples of viral antigens.

Exemplary Bacterial Antigens: Bacterial antigens can originate from any bacterium, including Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia. Antigens targeting bacteria can be derived from, for example, anthrax, gram-negative bacilli, chlamydia, diphtheria, Helicobacter pylori, Mycobacterium tuberculosis, pertussis toxin, pneumococcus, rickettsiae, Staphylococcus, Streptococcus and tetanus.

Bacterial infections against which the subject immunogenic constructs and methods may be used include both Gram-negative and Gram-positive bacteria. Examples of Gram-positive bacteria include Pasteurella spp., Staphylococci spp., and Streptococci spp. Examples of Gram-negative bacteria include Escherichia coli, Pseudomonas spp., and Salmonella spp.

Specific examples of infectious bacteria include Actinomyces israelii, Bacillus anthracis, Bacteroides spp., Borrella burgdorferi, pathogenic: Campylobacter spp., Clostridium perfringens, Clostridium tetani, Corynebacterium diphtheriae, Corynebacterium spp., Enterococcus spp., Enterobacter aerogenes, Erysipelothrix rhusiopathie, Escherichia coli, Fusobacterium nucleatum, Haemophilus influenzae, Helicobacter pylori, Klebsiella pneumoniae, Legionella pneumophilia, Leptospira, Listeria monocytogeners, Mycobacteria spp. (for example M. tuberculosis, M. avium, M. intracellilare, M. kansaii, M. gordonae), Neisseria gonorrhoeae, Neisseria meningitidis, Pasteurella multocida, Rickettsia, Shigella flexnerii, Shigella dysenteriae, Staphylococcus aureus, Streptococcus pyogenes (group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, Streptobacillus moniliformis, Treponerna pallidum, and Treponema pertenue, Vibrio cholerae

As particular examples of bacterial antigens, anthrax antigens include anthrax protective antigen; gram-negative bacilli antigens include lipopolysaccharides; diphtheria antigens include diphtheria toxin; Mycobacterium tuberculosis antigens include mycolic acid, heat shock protein 65 (HSP65), the 30 kDa major secreted protein and antigen 85A; pertussis toxin antigens include hemagglutinin, pertactin, FIM2, FIM3 and adenylate cyclase; pneumococcal antigens include pneumolysin and pneumococcal capsular polysaccharides; rickettsiae antigens include rompA; streptococcal antigens include M proteins; and tetanus antigens include tetanus toxin.

Exemplary Parasite Antigens: Parasite antigens can be obtained from any parasites, such as an antigen from Babesia microti, Babesi divergans, Candida albicans, Candida tropicalis, Chlamydial psittaci, Chlamydial trachomatis, Cryptococcus neoformans, Entamoeba histolytica, Giardia lamblia, Histoplasma capsulatum, Leishmania tropica, Leishmania spp., Leishmania braziliensis, Leishmania donovdni, Mycoplasma pneumoniae, Nocardia asteroides, Plasmodium falciparum, Plasmodium malariae, Plasmodium ovale, Plasmodium vivax, Rickettsia ricketsii, Rickettsia typhi, Schistosoma mansoni, Toxoplasma gondii, Trichomonas vaginalis, Trypanosoma brucei, Trypanosoma cruzi, Trypanosoma gambiense, Trypanosoma rhodesiense (African sleeping sickness), Trypanosoma cruzi (Chagos' disease), tinea (ringworm), flat worms, and round worms. The parasite may be a helminth organism or worm or organisms which cause diseases that include, but not limited to, Ancylostomiasis/Hookworm, Anisakiasis, Roundworm-Parasitic pneumonia, Roundworm-Baylisascariasis, Tapeworm-Tapeworm infection, Clonorchiasis, Dioctophyme renalis infection, Diphyllobothriasis-tapeworm, Guinea worm-Dracunculiasis, Echinococcosis-tapeworm, Pinworm-Enterobiasis, Liver fluke-Fasciolosis, Fasciolopsiasis-intestinal fluke, Gnathostomiasis, Hymenolepiasis, Loa loa filariasis, Calabar swellings, Mansonelliasis, Filariasis, Metagonimiasis-intestinal fluke, River blindness, Chinese Liver Fluke, Paragonimiasis, Lung Fluke, Schistosomiasis-bilharzia, bilharziosis or snail fever (all types), intestinal schistosomiasis, urinary schistosomiasis, Schistosomiasis by Schistosoma japonicum, Asian intestinal schistosomiasis, Sparganosis, Strongyloidiasis-Parasitic pneumonia, Beef tapeworm, Pork tapeworm, Toxocariasis, Trichinosis, Swimmers itch, Whipworm and Elephantiasis Lymphatic filariasis. The parasite may be an organism or organisms which cause diseases that include parasitic worm, Halzoun Syndrome, Myiasis, Chigoe flea, Human Botfly and Candiru. The parasite may be an ectoparasite or organisms which cause diseases that include Bedbug, Head louse-Pediculosis, Body louse-Pediculosis, Crab louse-Pediculosis, Demodex-Demodicosis, Scabies, Screwworm and Cochliomyia.

Antigens include Sporozoan antigens, Plasmodium antigens, such as all or part of a Circumsporozoite protein, a Sporozoite surface protein, a liver stage antigen, an apical membrane associated protein, or a Merozoite surface protein. Histoplasma antigens include heat shock protein 60 (HSP60); Leishmania antigens include gp63 and lipophosphoglycan; Plasmodium falciparum antigens include merozoite surface antigens, sporozoite surface antigens, circumsporozoite antigens, gametocyte/gamete surface antigens, protozoal and other parasitic antigens including the blood-stage antigen pf 155/RESA; schistosomae antigens include glutathione-S-transferase and paramyosin; toxoplasma antigens include SAG-1 and p30; and Trypanosoma cruzi antigens include the 75-77 kDa antigen and the 56 kDa antigen; tinea antigens include trichophytin.

Exemplary Fungal Antigens: Examples of fungal pathogens include Aspergillus spp., Blastomyces dermatitidis, Coccidoides immitis, Cryptococcus neoformans, Candida albicans and other Candida spp., Chlamydia trachomatis, Histoplasma capsulatum, Chlamydia trachomatis, Nocardia spp., and Pneumocytis carinii, Antigens targeting fungi can be derived from, for example, Candida, coccidiodes, Cryptococcus, and Histoplasma. As particular examples of fungal antigens, coccidiodes antigens include spherule antigens; and cryptococcal antigens include capsular polysaccharides.

As noted above, antigens from bacteria, virus, fungus and parasite may be formulated into the vaccines of the disclosure and administered according to the methods of the disclosure. Non-limiting examples of antigens include those form infectious agents that infect animals, such as the following:

Swine: Erysipelothrix rhusiopathiae, Actinobacillus pleuroneumonla, Mycoplasma hyopneumonlae, E. coli K88, K99, F41 and 987P, Clostridium perferingens type c, Salmonella choleraesuls, Pasterurella muitocida, Bordetella bronchiseptica, Leptospira bratislava, Leptospira canicola, Leptospira grippotyphosa, Leptospira hardjo, Leptospira promona, Leptospira ictero, Porcine Influenza virus, Circovirus, porcine reproductive and respiratory syndrome virus (PRRSV), Swine pox, Rotavirus, Porcine Respiratory Coronavirus, Parvo virus, Pseudorabies, transmissible gastroenteritis agent.

Horses: Streptococcus equi, Clostridium tetani, Equine Influenza Virus A1 and A2 strains, Equine Rhinopneumonids type 1, 1 b and 4, Eastern Equine Encephalomyelitis, Western Equine Encephalomyelitis, Venezuelan Equine Encephalomyelitis, Equine Rotavirus, Equine Herpesvirus, Equine Infectious Anemia Virus, West Nile Virus, Candida albicans, Aspergillus, Coccidioides immitis, Cryptococcus neoformans, Histoplasma farciminosum.

Cattle: E. coli O157:H7, Pasterurella multocida, Pasterurella haemolytica, Leptospira canicola, Leptospira grippotyphosa, Leptospira hardjo, Leptospira promona, Leptospira ictero, Clostridium perferingens type C, Clostridium perferingens type D, Clostridium chauvoel, Clostridium novyl, Clostridium septicum, Clostridium tetanus, Clostridium haemolyticum, Clostridium sodellii, Salmonella dublin and typhimurium, Bovine Rotavirus, Bovine coronavirus, Bovine rhinotracheitis, Bovine diarrhea virus, Parainfluenza-3, Respiratory syncytial virus, Trichophyton verrucosum.

Poultry: Salmonella typhimurium, Serpulina pilosicoli, Marek's disease virus, Infectious bursal disease, Infectious bronchitis, Newcastle disease virus, Reo virus, Turkey rhinotracheitis, coccidiosis.

Dogs: Leptospira canicola, Leptospira grippotyphosa, Leptospira hardjo, Leptospira promona, Leptospira ictero, Canine Borrella burgdorferi, Canine Ehrlichia canis, Canine Bordetella bronchiseptica, Canine Giardia lamblia, Canine distemper, Canine Adenovirus, Canine Coronavirus, Canine Parainfluenza, Canine Parvovirus, Canine Rabies, Fleas, Giardia, Lungworms, Ancylostoma caninum, Uncinaria stenocephala, Microsporum canis.

Cats: Feline infectious peritonitis virus, Feline rhinotrachelitis, Feline Panleukopenia, Feline calicivirus, Feline coronavirus, Feline alphaherpesvirus 1, Feline immunodeficiency virus, Feline leukemia virus, rabies lyssavirus. Bordetella, Bacillus, Bartonella, Burkholderia, Chlamydia, Clostridium, Corynebacterium, Salmonella, Proteus, Escherichia, Proteus, Moraxella, Nocardia, Pasteurella, Haemophilus, Pasteurella, Pseudomonas, Staphylococcus, Streptococcus, Microsporum canis, Nannizzia gyps, Nannizzia fulva, Nannizzia nana, Trichophyton mentagrophytes, Trichophyton verrucosum, Ancylostoma caninum, Cryptosporidium, Dirofilaria immitis, Fleas, Giardia, Isospora sp., Lungworms, Ollanulus tricuspi, Physaloptera hispida, Sarcoptes scabiei, Tapeworms, Toxascaris leonina, Toxocara cati, Toxoplasma gondii, Uncinaria stenocephala, and Whipworms.

### VII. Oligonucleotides

In some embodiments, the immunogenic construct includes one or more oligonucleotides, e.g., siRNA, miRNA, miRNA mimics, or antisense oligonucleotides. Oligonucleotides may be attached by any means. In some embodiments, a negatively charged siRNA is attached to the positively charged cationic polymer on the nanoparticle, e.g., MSNP, using an electrostatic interaction. The oligonucleotides may target one or more genes expressed in a cell, e.g., one that inhibits or downregulates genes associated with immunosuppression of an antigen-presenting cell (e.g. dendritic cell), such as STAT3, PD-L1, IDO-1, and IL-6. In some embodiments, a single oligonucleotide may target a plurality of genes with varying potency. In other embodiments, a plurality of oligonucleotides may target a single gene. In further embodiments, a plurality of oligonucleotides may target a plurality of genes.

Oligonucleotides may be present at about 1% to 10% by weight of the NP, e.g., about 2% to about 6% by weight. In some embodiments, For example, NP per siRNA (NP/siRNA) is used at the weight ratio ranging between about 10:1 to about 100:1 during the binding process, achieving complete binding. Complete binding can be achieved up to 40 wt. % siRNA per NP.

Typically, the oligonucleotide inhibits or downregulates genes whose upregulation is associated with some aspect of immunosuppression of antigen-presenting cells (e.g., dendritic cells). One of ordinary skill in the art will understand how to access representative sequences for these targets, which are readily available in public sequence databases. In some embodiments, the oligonucleotide is a siRNA such as STAT3, PD-L1, IDO-1, IL-6, etc. Exemplary siRNAs are shown in Table 2.

TABLE 2

| | | siRNA sequences |
|---|---|---|
| Name | Representative GenBank Accession No. | SEQ ID NOs: |
| Signal transducer and activator | NM_003150.3; NM_139276.2; NM_213662.1; | 9 and 10 |
| of transcription 3 | XM_005257616.3; XM_005257617.3; XM_011525145.2; | |
| (STAT3) | XM_011525146.2; XM_017024972.1; XM_017024973.1; | |
| | XM_017024974.1; XM_017024975.1; XM_017024976.1 | |
| Programmed death- | NM_001267706.1; NM_001314029.2; NM_014143.4; | 11-14 |
| ligand 1 (PD-L1) | NR_052005.2 | |
| Programmed death- | NM_025239.4; XM_005251600.3; XP_005251657.1 | 15-18 |
| ligand 2 (PD-L2) | | |
| Indoleamine 2,3- | NM_002164.6; NG_028155.1 | 19-22 |
| dioxygenase 1 (IDO-1) | | |
| Suppressor of | NM_003745.1; DQ086801.1; U88326.1; NP_003736.1 | 23-29 |
| cytokine signaling 1 | | |
| (SOCS-1) | | |
| Transforming growth | NM_000660.7; XM_011527242.2; | 30 and 31 |
| factor beta 1 (TGF-β) | | |
| TGF beta receptor 1 | NM_004612.4; NM_001130916.3; NM_001306210.2; | 32-36 |
| (TGFBR1) | XM_011518948.2; XP_011517250.1; XM_011518949.2; | |
| | XP_011517251.1 | |
| TGF beta receptor 2 | NM_003242.6; NM_001024847.2; XM_011534043.2; | 37-40 |
| (TGFBR2) | XP_011532345.1; XM_011534045.3; XP_011532347.1; | |
| | XM_017007106.1; XP_016862595.1 | |
| Interleukin-10 (IL-10) | NG_012088.1; NM_000572.3; NM_001382624.1; | 41-45 |
| | NR_168466.1; NR_168467.1 | |
| Interleukin 10 receptor | NM_001558.4; NR_026691.2; XM_024448493.1; | 46 and 47 |
| subunit alpha (IL- | XP_024304261.1 | |
| 10RA) | | |
| Arginase-1 (ARG-1) | NG_007086.2; NM_000045.4; NM_001244438.2; | 48-51 |
| | NM_001369020.1; NR_160934.1 | |
| Galectin-1 (LGALS1) | NM_002305.4; NP_002296.1 | 52-54 |
| Galectin-3 (LGALS3) | NG_017089.1; NM_001357678.2; NP_001344607.1; | 55-51 |
| | NM_002306.4; NP_002297.2; NR_003225.2 | |
| lnterleukin-6 (IL-6) | NG_011640.1; NM_000600.5; NM_001318095.2; | 62-66 |
| | NP_001305024.1; NM_001371096.1; NP_001358025.1 | |
| V-set domain containing | NM_024626.4; NP_078902.2; NM_001253849.1; | 67 and 68 |
| T cell activation inhibitor | NP_001240778.1; NM_001253850.1; NP_001240779.1 | |
| (VTCN-1) | | |
| Inducible nitric oxide | NG_011470.1; NM_000625.4; NP_000616.3 | 69-75 |
| synthase (iNOS, or NOS2) | | |
| Semaphorin-4A | NM_022367.4; NM_001193300.2; NM_001193301.1; | |
| (SEMA4A) | NM_001193302.1; NP_001180231.1; NM_001370567.1 | |
| Bcl-2 homologous | NM_001188.4; NP_001179.1; XM_011514779.3; | 76-78 |
| antagonist killer | XP_011513081.1; XM_011514780.1; XP_011513082.1 | |
| (BAK1) | | |
| Bcl-2-associated X | NM_138761.4; NP_620116.1; NM_004324.4; | 79-81 |
| protein (BAX) | NP_004315.1; NM_138763.4; NP_620118.1; | |
| | NM_138764.5; NP_620119.2; NM_001291428.2; | |
| | NP_001278357.1 | |
| Bcl-2-like protein 11 | NM_138621.5; NP_619527.1; NM_006538.5; | 82-85, 91 |
| (BIM, or BCL2L11) | NP_006529.1; NM_138622.3; NP_619528.1; | |
| | NM_138623.3; NP_619529.1; NM_138624.3; | |
| | NP_619530.1 | |
| (Phosphatase and | NM_000314.8; NP_000305.3; NM_001304717.5; | 86-89 |
| tensin homolog) PTEN | NP_001291646.4; NM_001304718.2; NP_001291647.1 | |

### VIII. Targeting Agents

In some embodiments, the immunogenic construct may further include a targeting agent, e.g., for specific delivery of the immunogenic constructs to a target site. Targeting agents may be used to target a site and optionally to aid or induce internalization into a cell.

Exemplary targeting agents include monoclonal antibodies, single chain variable fragment (scFv) antibodies, other antigen binding fragments of antibodies, aptamers, small targeting molecules (e.g., ligands that bind to cell surface receptors such as N-acetylgalactosamine, mannose, transferrin, and folic acid), aptamers, carbohydrates, and peptides that have binding affinity to a cell or tissue, e.g., an immune cell such as an antigen-presenting cell (e.g., a dendritic cell or a macrophage).

In some embodiments, the targeting agent targets an immune cell such as an antigen-presenting cell (e.g., a dendritic cell or a macrophage). Targeting agents include monoclonal or polyclonal antibodies or fragments thereof that recognize and bind to epitopes displayed on the surface of the immune cell, and ligands which bind to a cell surface receptor on the immune cell. One such receptor, the lectin DEC-205, has been used in vitro and in mice to boost both humoral (antibody-based) and cellular (CD8 T cell) responses by 2-4 orders of magnitude (Hawiger et al., J. Exp. Med., 194(6):769-79, 2001; Bonifaz et al., J. Exp. Med., 196(12):1627-38 2002; Bonifaz et al., J. Exp. Med., 199(6):815-24, 2004). In these reports, antigens were fused to an anti-DEC205 heavy chain and a recombinant antibody molecule was used for immunization.

A variety of other endocytic receptors, including a mannose-specific lectin (mannose receptor) and IgG Fc receptors, have also been targeted in this way with similar enhancement of antigen presentation efficiency. Other suitable receptors and surface proteins which may be targeted include DC-SIGN, 33D1, SIGLEC-H, DCIR, CD11c, CD40, DEC-205, heat shock protein receptors, and scavenger receptors. Targeting moieties for these receptors can be attached to the immunogenic constructs for their preferential uptake into immune cells that express these receptors. Example is mannose attached on the immunogenic constructs for targeted delivery to macrophages and DCs that have high levels of mannose receptors.

Other receptors which may be targeted include the toll-like receptors (TLRs). TLRs recognize and bind to pathogen-associated molecular patterns (PAMPs). PAMPs target the TLR on the surface of the dendritic cell and signals internally, thereby potentially increasing DC antigen uptake, maturation and T-cell stimulatory capacity. PAMPs conjugated to the particle surface or co-encapsulated include unmethylated CpG DNA (bacterial), double-stranded RNA (viral), lipopolysaccharide (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

The targeting agents may be attached to the immunogenic constructs by any means, and suitable conjugation chemistries are known in the art and described herein. In some embodiments, the targeting agent is thiolated and subsequently conjugated with Mal-PEG-PEI-MSNP via a thiol-maleimide reaction. In some embodiments, the targeting agent is attached to a PEG stabilizer prior to conjugation to the NP by reaction of an NHS ester and an amine. The targeting agent may be present at 0.1 to 10 wt. % of the NP, e.g., 0.1 to 1 wt. % or 1 to 5 wt. %, e.g., 1 to 10 wt. % for antibody or 0.1 to 2 wt. % for scFV, e.g., about 1, 2, 3, 4, 5, 6, 7, 8, or 9 wt. %.

### IX. Labeling Agents

In some embodiments, the immunogenic construct may be labeled, e.g., with a lanthanide, a fluorescent dye, a quantum dot, a radiotracer, or a gold nanoparticle. A label may be any substance capable of aiding a machine, detector, sensor, device, column, or enhanced or unenhanced human eye from differentiating a labeled composition from unlabeled compositions. Examples of labels include radioactive isotopes (e.g., PET tracers), dyes, stains, quantum dots, gold nanoparticles, enzymes, nonradioactive metals (e.g., MRI contrast agents), magnets, biotin, protein tags, any antibody epitope, or any combination thereof. Exemplary fluorescent dyes include FITC, RITC, Cy™ dyes, amine-reactive Dylight® dyes, and amine-reactive Alexa Fluor® dyes. In some embodiments, lanthanides can be loaded onto hydroxyl, thiol, amine or phosphonate groups of nanoparticles, e.g., MSNPs, by covalent bonding or adsorption. Lanthanides can facilitate sample detection with high sensitivity and resolution, e.g., by mass spectrometry, while fluorescent dyes permit sample quantification by fluorescent imaging techniques. Immunogenic constructs containing lanthanides such as gadolinium can also serve as MRI contrast agents for imaging disease sites.

In some embodiments, the labels, such as fluorescent dyes, are loaded inside pores of nanoparticles, e.g., amine-MSNPs via nucleophilic acyl substitution, e.g., between one or more nanoparticle-bound amines and an activated ester moiety (such as an NHS ester) appended to a fluorescent dye. Such labels produce immunogenic constructs that may be tracked using fluorescence imaging techniques. Such a label may be added prior to or after loading of the cationic polymer and/or stabilizer (that is, the label may be applied to the nanoparticle, or to the NP). In further embodiments, the label may be attached to the cationic polymer, stabilizer, or other component (e.g., the oligonucleotide) of the NP prior to or after their attachment to the nanoparticle, by any appropriate means.

### X. Immunogenic Construct Synthesis

Components may be bound to nanoparticles or other components of the NP or immunogenic constructs by any means, including covalent and electrostatic binding. Various conjugation chemistries are known in the art and described herein. In some embodiments, one or more of the components are bound to the surface of the nanoparticles or NPs. In other embodiments, one or more of the components are bound within the pores of the nanoparticle (e.g., MSNP). In further embodiments, one or more of the components are bound to each other. In some embodiments, an adjuvant and/or an antigen or antigen producing agent is covalently bound to a stabilizer. The stabilizer may be covalently bound to the cationic polymer (e.g., via an amine), which may be, in turn, electrostatically bound to the exterior of the nanoparticle. In some embodiments, an adjuvant and/or an antigen or antigen producing agent is bound to the cationic polymer via chemical conjugation, electrostatic interaction, hydrophobic interaction, hydrogen bonding, or van der Waals interactions. For example, an antigen or antigen producing agent may be covalently bound to the stabilizer, while an adjuvant is electrostatically or hydrophobically bound to the cationic polymer.

In some embodiments in which the nanoparticle has a pore, the pore has a first opening at a first location on the exterior surface of the nanoparticle (e.g., MSNP) and a second, different opening at a second location on the exterior surface of the nanoparticle. Components may be bound anywhere along the length of the inside of the pore, though the size of the pore and the size of the component will influence binding.

While nanoparticles, such as MSNPs, may be acquired commercially or created by any method, in some embodiments MSNPs are formed by combining a first surfactant with a second, different surfactant to form a first mixture, heating up the first mixture and adding a silica precursor to the first mixture to form a second mixture, holding the temperature for a period of time to generate MSNPs, and recovering the MSNPs by centrifugation. Surfactants can be removed by mixing the MSNP in acidic solvent under reflux conditions. In some embodiments, the first mixture may be heated prior to adding the silica precursor. In other embodiments, the first mixture may be at room temperature and the second mixture may be heated. The resulting MSNPs may have uniform or non-uniform particle size with high porosity.

For example, to form uniform MSNPs, cetyltrimethylammonium chloride (CTAC) may be combined with triethanolamine (TEA) in water, and heated to 95° C., while tetraethyl orthosilicate is added. Variation of the amount of TEA while holding the amount of CTAC constant can be used to alter the size of the resulting MSNPs. In some embodiments, the amount of TEA is between about 100 to about 600 μL, about 200 to about 450 μL, or about 200 to about 350 μL. In some embodiments, the amount of TEA is 0.1-1% v/v, e.g., 0.35% v/v. Non-uniform MSNPs may be created using a strong base, such as NaOH. For example, cetyltrimethyl ammonium bromide (CTAB) may be used as the surfactant and NaOH may be used as the base catalyst.

Iron oxide nanoparticles can be purchased (e.g., Feraheme) or synthesized. Gold and silver nanoparticles can be synthesized following various published protocols or purchased from vendors such as Sigma Aldrich, Nanocs, nanoComposix. Carbon nanotubes can be synthesized following various published protocols or purchased from vendors such as Sigma Aldrich, US Research nanomaterial, and American Elements.

In some embodiments, functional groups such as, but not limited to, thiol, amine, carboxylate, or phosphonate may be added to exterior surface of the nanoparticles, e.g., MSNPs, during synthesis through the use of one or more reagents, e.g., organosilanes such as (3-aminopropyl)triethoxysilane and (3-aminopropyl)trimethoxysilane). Organosilanes may be added before or after the surfactants are removed from the MSNPs. Analogous reagents and other organic reagents, such as glutathione, mercaptopropionic acid, DMSA, PEG-thiol, oleic acid, and dextran may be employed to modify iron oxide nanoparticles, silver nanoparticles, gold nanoparticles, and carbon nanotubes. Functionalized nanoparticles can also be purchased directly, e.g., carbon nanotubes having a surface modified with carboxylic acid, amide, polyaminobenzene sulfonic acid, octadecylamine, and PEG can be purchased from Sigma Aldrich.

The resulting NPs (e.g., MSNPs after surface modification) may be of any appropriate size, e.g., from about 20 nm to about 200 nm, about 20 nm to about 400 nm, about 20 nm to about 500 nm, about 20 nm to about 100 nm, about 30 nm to about 100 nm, about 40 nm to about 200 nm, about 50 nm to about 200 nm, about 50 nm to 400 nm, about 50 to 500 nm, about 30 nm to about 80 nm, 40 nm to about 80 nm, about 30 nm, about 40 nm, about 30 nm to about 60 nm, about 50 nm, about 60 nm, about 80 nm, about 100 nm, about 120 nm, or about 150 nm.

The resulting immunogenic constructs, e.g., NPs loaded with antigen(s) or antigen producing agent(s), may be of appropriate size, e.g., from about 20 nm to about 200 nm, about 30 nm to about 100 nm, about 40 nm to about 200 nm, about 50 nm to about 200 nm, about 30 nm to about 80 nm, 40 nm to about 80 nm, about 30 nm, about 40 nm, about 30 nm to about 60 nm, about 100 nm to 200 nm, about 100 nm to about 500 nm, about 100 nm to about 999 nm, about 100 nm to about 400 nm, about 50 nm, about 60 nm, about 80 nm, about 100 nm, about 120 nm, about 150 nm, about 200 nm, about 300 nm, about 400 nm, about 500 nm, about 600 nm, about 700 nm, about 800 nm, about 900 nm, about 999 nm.

### XI. Immunogenic Constructs Including Lipid-Coated Nanoparticles

Also disclosed herein are immunogenic constructs including lipid coated nanoparticle cores (e.g., any of the nanoparticles disclosed herein). In some embodiments, the immunogenic construct includes a lipid-coated calcium phosphate is composed of a calcium phosphate core (CaP-L) in which the core is, e.g., formed by the reaction between CaCl₂) and Na₂HPO₄ and a surrounding lipid layer. The Ca/P molar ratio may range from 10 to 200. The size of the CaP core nanoparticle may range from 5 to 999 nm (e.g., about 20 nm to about 200 nm, about 30 nm to about 100 nm, about 40 nm to about 200 nm, about 50 nm to about 200 nm, about 30 nm to about 80 nm, 40 nm to about 80 nm, about 30 nm, about 40 nm, about 30 nm to about 60 nm, about 50 nm, or about 60 nm). The lipid layer thickness may range from 1 to 999 nm (e.g., about 20 nm to about 200 nm, about 30 nm to about 100 nm, about 40 nm to about 200 nm, about 50 nm to about 200 nm, about 30 nm to about 80 nm, 40 nm to about 80 nm, about 200 to about 750 nm, about 500 to 999 nm about 30 nm, about 40 nm, about 30 nm to about 60 nm, about 50 nm, or about 60 nm). The lipid layer includes one or more of a cationic lipid (e.g., DOTAP, dimethyldioctadecylammonium bromide, D-Lin-MC3-DMA), a PEGylated lipid (e.g., DMG-PEG 2000, DSG-PEG 2000), a functionalized PEGylated lipid with functional groups (e.g., —SH, —NH₂, —COOH), a PEGylated lipid conjugated with a targeting agent (e.g., mannose or any of those disclosed herein), a phospholipid (e.g., 1,2-distearoyl-sn-3-phosphacholine (DSPC), dioleoylphosphatidic acid (DOPA), or dioleoylphosphatidylethanolamine (DOPE)), and cholesterol. Each of the above lipids may account for 0-100% (w/w) (e.g., 0-10%, 0-20%, 0-30%, 0-40%, 0-50%, 0-60%, 0-70%, 0-80%, 0-90%, 5-15%, 5-25%, 10-50%, 25-75%, 50-90%, or about 10%, about 20%, about 30%, about 40%, about 50%, about 60%, about 70%, about 80%, about 90%, or 100%) of the lipid layer. While the PEGylated lipid/functionalized PEGylated lipid/targeted PEGylated lipid enhance the stability and prolong the circulation of the immunogenic construct in blood, the phospholipid composition and cholesterol form and stabilize the lipid coating structure. The functionalized PEGylated lipid is for further conjugation with nucleic acids and/or antigens. The targeted PEGylated lipid is for enhancing uptake efficacy into targeted cells. The calcium phosphate core may account for 0.1-99.9% (w/w) of the CaP-L (e.g., 0-10%, 0-20%, 0-30%, 0-40%, 0-50%, 0-60%, 0-70%, 0-80%, 0-90%, 5-15%, 5-25%, 10-50%, 25-75%, 50-90%, or about 10%, about 20%, about 30%, about 40%, about 50%, about 60%, about 70%, about 80%, about 90%, or 99.9%). The lipid layer may account for 0.1-99.9% (w/w) of the CaP-L (e.g., 0-10%, 0-20%, 0-30%, 0-40%, 0-50%, 0-60%, 0-70%, 0-80%, 0-90%, 5-15%, 5-25%, 10-50%, 25-75%, 50-90%, or about 10%, about 20%, about 30%, about 40%, about 50%, about 60%, about 70%, about 80%, about 90%, or 99.9%). One or more types of surfactants (e.g., Tween 80, Tween 20, Span 80, Span 20, PVP, SDS, SLS, PEG) can be included to help the formation of CaP-L. The surfactant:CaP-L weight ratio can be in the range of 0-50%. The hydrodynamic size of CaP-L with/without loaded cargo can be in the range of 10 nm to 10 microns (e.g., about 80 nm to about 200 nm, or about 90 nm to about 150 nm, about 1 micron to about 2 micron).

In some embodiments, one or more types of oligonucleotides (e.g., one or more of a siRNA, a mRNA, a shRNA, a miRNA, a DNA, and a CpG oligonucleotide) are loaded into the core of the nanoparticle (e.g., a calcium phosphate core through the ionic interaction between Ca²⁺ cations and phosphate groups of nucleic acid backbones of the oligonucleotides). In some embodiments, one or more types of oligonucleotides (e.g., one or more of a siRNA, a mRNA, a shRNA, a miRNA, a DNA, and a CpG oligonucleotide) are loaded into a lipid layer by the ionic interaction between cationic lipids and phosphate groups of the nucleic acid backbones. In some embodiments, one or more types of oligonucleotides (e.g., one or more of a siRNA, a mRNA, a shRNA, a miRNA, a DNA, and a CpG oligonucleotide) are conjugated to functionalized PEGylated lipids of a lipid layer. The loading of the one or more types of oligonucleotides, e.g., into CaP-L, can be in the range of 0.01 to 10 wt. % of the nanoparticle.

In some embodiments, one or more types of antigens or antigen producing agents (e.g., one or more of a peptide, a protein, and a polysaccharide) are loaded into the core of the nanoparticle (e.g., a calcium phosphate core). In some embodiments, one or more types of antigens or antigen producing agents (e.g., one or more of a peptide, a protein, and a polysaccharide) with appropriate hydrophilic-lipophilic balances are inserted into a lipid layer. In some embodiments, one or more types of antigens or antigen producing agents (e.g., one or more of a peptide, a protein, and a polysaccharide) are adsorbed on the surface of the nanoparticle (e.g., a calcium phosphate nanoparticle via Van der Waals interaction and/or ionic interaction with the Ca²⁺ ions of the calcium phosphate core). In some embodiments, one or more types of antigens or antigen producing agents (e.g., one or more of a peptide, a protein, and a polysaccharide) can be conjugated to functionalized PEGylated lipids of a lipid layer through a covalent bond. The loading of the one or more types of antigens or antigen producing agents into a nanoparticle (e.g., a calcium phosphate nanoparticle) can be in the range of 0.01 to 10 wt. % of the nanoparticle.

### XII. Immunogenic Construct Formulations and Methods of Use

Immunogenic constructs may be formulated, as is known in the art, for therapeutic, diagnostic, or research use. Immunogenic constructs may be employed for in vivo or ex vivo use. Effects of the agents contained in the immunogenic construct may occur intracellularly or extracellularly.

The immunogenic constructs may be used immediately upon formulation or may be stored. In some embodiments, the immunogenic constructs may be lyophilized into dry states using a lyoprotectant, such as a sugar like trehalose. Optimal trehalose and lyophilization conditions may preserve the immunogenic construct in terms of particle size and charge and efficacy, e.g., in terms of gene knock down efficacy for immunogenic constructs containing certain siRNAs, compared to freshly made material. Immunogenic constructs of the disclosure are stable for at least 6 months when lyophilized.

Immunogenic constructs may be formulated with a pharmaceutically effective excipient in a pharmaceutical composition. Pharmaceutical compositions may include active agents, e.g., adjuvants that are not bound to the immunogenic construct, lyoprotectants, stabilizing agents, preservatives, and/or solubilizing agents. Effective amounts of an immunogenic construct for therapeutic administration will be readily determined by those of ordinary skill in the art, depending for instance on clinical and patient-specific factors.

These and other effective unit dosage amounts may be administered in a single dose, or in the form of multiple daily, weekly or monthly doses, for example in a dosing regimen of twice per week for a 3-week cycle. In additional embodiments, dosages may be administered in concert with other treatment regimens in any appropriate dosage regimen depending on clinical and patient-specific factors. The amount, timing and mode of delivery of compositions of the disclosure comprising an immunogenic amount of an immunogenic construct will be routinely adjusted on an individual basis, depending on such factors as weight, age, gender, and condition of the individual, the acuteness of the disease and/or related symptoms, whether the administration is prophylactic or therapeutic, and on the basis of other factors known to effect drug delivery, absorption, pharmacokinetics including half-life, and efficacy.

Formulations of the disclosure will ordinarily be selected to approximate a minimal dosing regimen that is necessary and sufficient to substantially prevent or alleviate the symptoms of the disease including cancer, fibrosis and inflammation in the mammalian subject, including humans. Therapeutic dosage and administration protocol will often include repeated dosing over a course of several days or even one or more weeks or years. An effective treatment regimen may also involve prophylactic dosage administered on a day or multi-dose per day basis lasting over the course of days, weeks, months or even years.

In some embodiments, the immunogenic constructs of the disclosure are formulated for parenteral administration, e.g. to be administered intravenously, intramuscularly, intratumorally, intranasally, subcutaneously, intradermally, or intraperitoneally, including aqueous and non-aqueous sterile injectable solutions which, like many other contemplated compositions of the disclosure, may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Additional compositions and formulations of the disclosure may include polymers for extended release following parenteral administration. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such materials are well known to those of ordinary skill in the pharmaceutical compounding arts. Parenteral preparations typically contain buffering agents and preservatives, and injectable fluids that are pharmaceutically and physiologically acceptable such as water, physiological saline, balanced salt solutions, aqueous dextrose, glycerol or the like. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In some embodiments, the immunogenic constructs are formulated for oral administration and can be in ay orally acceptable dosage form including capsules, tablets, emulsions and aqueous suspensions, dispersions, and solutions. In some embodiments, the dosage form is an oral dosage form such as a pressed tablet, hard or soft gel capsule, enteric coated tablet, osmotic release capsule, or unique combination of excipients. In the case of tablets, commonly used excipients include lactose, mannitol, and corn starch. Lubricating agents, such as, but not limited to, magnesium stearate, also are typically added. For oral administration in a capsule form, useful diluents include lactose, mannitol, glucose, sucrose, corn starch, potato starch, or cellulose. In additional embodiments, the dosage form includes a capsule wherein the capsule contains a mixture of materials to provide a desired sustained release formulation. When aqueous suspensions or emulsions are administered orally, the active ingredient can be suspended or dissolved in an oily phase combined with emulsifying or suspending agents. If desired, certain sweetening, flavoring, or coloring agents can be added.

In some embodiments, the immunogenic constructs are formulated for intranasal administration or inhalation. Compositions for nasal administration or inhalation may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluoro-chloro-hydrocarbon. The aerosol dosage forms can also take the form of a pump-atomizer.

A topical carrier may be used to deliver the immunogenic construct. In some embodiments, the topical carrier is an emulsion, a gel, or an ointment. In other embodiments, the immunogenic constructs may be formulated in a spray formulation. Emulsions, such as creams and lotions are a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase as droplets, the emulsion is termed an oil-in-water emulsion. Emulsions, such as creams and lotions, that can be used as topical carriers and their preparation are disclosed in Remington: The Science and Practice of Pharmacy (Loyd V. Allen 22^{nd} ed. 2012), hereby incorporated herein by reference.

Ointments may be homogeneous, viscous, semi-solid preparation, most commonly a greasy, thick oil (oil 80%-water 20%) with a high viscosity. The ointment can be used as an emollient or for the application of active ingredients to the skin for protective, therapeutic, or prophylactic purposes where a degree of occlusion is desired.

A cream is an emulsion of oil and water in approximately equal proportions. It penetrates the stratum corneum outer layer of skin quite well. A cream is generally thinner than an ointment, and maintains its shape when removed from its container.

The vehicle of an ointment/cream is known as the ointment base. The choice of a base depends upon the clinical indication for the ointment. Different types of ointment bases include: hydrocarbon bases, e.g. hard paraffin, soft paraffin, microcrystalline wax and ceresine; absorption bases, e.g., wool fat, beeswax; water-soluble bases, e.g., macrogols 200, 300, and 400; and emulsifying bases, e.g. emulsifying wax, vegetable oils (such as olive oil, coconut oil, sesame oil, almond oil and peanut oil). The immunogenic constructs are dispersed in the base and later get divided after the drug penetrates into the wound. Ointments/creams can be formulated incorporating hydrophobic, hydrophilic, or water-emulsifying bases to provide preparations that are immiscible, miscible, or emulsifiable with skin secretions. They can also be derived from fatty hydrocarbon, absorption, water-removable, or water-soluble bases. For example, a cream/ointment base can contain the active agent, white petrolatum, water, allantoin, EDTA, Stearyl alcohol, Brij 721, Brij 72, methylcelluloses, isopropyl myristate, Sorbitan monooleate, Polyoxyl 40 stearate, butylated hydroxytoluene, propylene glycol, methylparaben, propylparaben, deionized water to 100%, and buffer to neutral pH among other ingredients.

In another embodiment, the topical carrier used to deliver an immunogenic construct of the disclosure is a gel, for example, a two-phase gel or a single-phase gel. Gels are semisolid systems consisting of suspensions of small inorganic particles or large organic molecules interpenetrated by a liquid. When the gel mass includes a network of small discrete inorganic particles, it is classified as a two-phase gel. In some embodiments, the liquid may be water or another aqueous media and the gel mass is defined as a hydrogel. Hydrogels can include alginates, polyacrylates, polyalkylene oxides, and/or poly N-vinyl pyrrolidone. The hydrogel may also be amorphous, i.e., a viscous gel as opposed to a solid such as a formulation of carboxymethylcellulose containing a humectant such as propylene glycol or glycerin. Exemplary amorphous hydrogels include maltodextra-β-glucan, acemannan, carboxymethylcellulose, pectin, xanthan gum, collagen, keratin, and honey.

Immunogenic constructs may be packaged into biodegradable capsules for oral administration. Alternatively, an immunogenic construct suspension may be installed inside the bladder. This is similar to intravesical chemotherapy, in which the drug administered to the bladder will come into direct contact with cancer cells in the bladder lining.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure. In the following examples, and for the purposes of explanation, numerous specific details are set forth in order to provide a thorough understanding of the various aspects of the disclosure. It will be understood, however, by those skilled in the relevant arts, that the present disclosure may be practiced without these specific details. In other instances, known structures and devices are shown or discussed more generally in order to avoid obscuring the invention. It should be noted that there are many different and alternative configurations, devices and technologies to which the disclosed inventions may be applied. The following examples are illustrative of disclosed methods. In light of this disclosure, those of skill in the art will recognize that variations of these examples and other examples of the disclosed method would be possible without undue experimentation.

### XIII. Exemplary Embodiments

### Exemplary Embodiments Set 1

1. An immunogenic construct including: a nanoparticle; a crosslinked cationic polymer bound to an exterior surface of the nanoparticle; a stabilizer bound to the crosslinked cationic polymer or the exterior surface of the nanoparticle; and an antigen or an antigen producing agent for an infectious agent.
2. The immunogenic construct of embodiment 1, further including an adjuvant.
3. The immunogenic construct of embodiment 2, wherein the adjuvant includes one or more of a CpG oligonucleotide, a DNA TLR agonist containing a CpG sequence, a non-CpG DNA TLR agonist, an RNA TLR agonist, an aluminum salt, an anti-CD40 antibody, a fusion protein, a cytokine, a small molecule TLR agonist, an oil- or surfactant-based adjuvant, a lipopolysaccharide, a plant extract, or a derivative thereof.
4. The immunogenic construct of embodiment 2 or 3, wherein the adjuvant includes a CpG oligonucleotide.
5. The immunogenic construct of any one of embodiments 2-4, wherein the adjuvant includes poly I:C.
6. The immunogenic construct of any one of embodiments 2-5, wherein the adjuvant is present at 1-20 wt. % of the nanoparticle.
7. The immunogenic construct of any one of embodiments 1-6, wherein the nanoparticle is a silica nanoparticle, a silicon nanoparticle, an iron oxide nanoparticle, a gold nanoparticle, a silver nanoparticle, a carbon nanotube, or an adjuvant nanoparticle.
8. The immunogenic construct of any one of embodiments 1-7, wherein the nanoparticle is a mesoporous silica nanoparticle.
9. The immunogenic construct of any one of embodiments 1-8, wherein the cationic polymer is selected from the group consisting of PEI, chitosan, polypropyleneimine, polylysine, polyamidoamine, poly(allylamine), poly(diallyldimethylammonium chloride), poly(N-isopropyl acrylamide-co-acrylamide), poly(N-isopropyl acrylamide-co-acrylic acid), diethylaminoethyl-dextran, poly-(N-ethyl-vinylpyridinium bromide), poly(dimethylamino)ethyl methacrylate, and poly(ethylene glycol)-co-poly(trimethylaminoethylmethacrylate chloride).
10. The immunogenic construct of any one of embodiments 1-9, wherein the cationic polymer is PEI.
11. The immunogenic construct of any one of embodiments 1-10, wherein the cationic polymer has a molecular weight of about 0.8 kDa to about 25 kDa.
12. The immunogenic construct of any one of embodiments 1-11, wherein the cationic polymer is present at 1-50 wt. % of the nanoparticle.
13. The immunogenic construct of any one of embodiments 1-12, wherein the stabilizer is selected from the group consisting of PEG, dextran, polysialic acid, hyaluronic acid, polyvinyl pyrrolidone, polyvinyl alcohol, and polyacrylamide.
14. The immunogenic construct of any one of embodiments 1-13, wherein the stabilizer is PEG.
15. The immunogenic construct of any one of embodiments 1-14, wherein the stabilizer has a molecular weight of about 1 kDa to about 20 kDa.
16. The immunogenic construct of any one of embodiments 1-15, wherein the stabilizer is present at 1-50 wt. % of the nanoparticle.
17. The immunogenic construct of any one of embodiments 1-16, wherein the infectious agent is a virus.
18. The immunogenic construct of any one of embodiments 1-17, wherein the infectious agent is a beta-coronavirus.
19. The immunogenic construct of any one of embodiments 1-18, wherein the infectious agent is SARS-CoV-2, a SARS-CoV-1, or MERS-CoV.
20. The immunogenic construct of any one of embodiments 1-19, wherein the infectious agent is SARS-CoV-2.
21. The immunogenic construct of embodiment 20, wherein the antigen or antigen producing agent is a recombinant full-length protein.
22. The immunogenic construct of embodiment 21, wherein the antigen or antigen producing agent is a full-length SARS-CoV-2 spike glycoprotein, a SARS-CoV-2 nucleocapsid protein, or a SARS-CoV-2 membrane protein.
23. The immunogenic construct of embodiment 20, wherein the antigen or antigen producing agent is a protein subunit.
24. The immunogenic construct of embodiment 23, wherein the antigen or antigen producing agent is a protein subunit corresponding to the S1, S2, or RBD region of the SARS-CoV-2 spike glycoprotein.
25. The immunogenic construct of embodiment 20, wherein the antigen or antigen producing agent is a peptide corresponding to an immunogenic sequence of SARS-CoV-2 spike glycoprotein.
26. The immunogenic construct of embodiment 25, wherein the antigen or antigen producing agent has the peptide sequence of any one of SEQ ID NOs: 1-8.
27. The immunogenic construct of embodiment 20, wherein the antigen or antigen producing agent is a mRNA or a pDNA.
28. The immunogenic construct of any one of embodiments 1-27, wherein the antigen or antigen producing agent is present at 0.5-20 wt. % of the nanoparticle.
29. The immunogenic construct of any one of embodiments 1-28, wherein the immunogenic construct further includes at least one type of oligonucleotide
30. The immunogenic construct of embodiment 29, wherein the at least one type of oligonucleotide is electrostatically bound to the cationic polymer.
31. The immunogenic construct of embodiment 30, wherein the at least one type of oligonucleotide includes a siRNA, a miRNA, a miRNA mimic, or an antisense oligonucleotide.
32. The immunogenic construct of embodiment 31, wherein the at least one type of nucleotide includes a siRNA.
33. The immunogenic construct of embodiment 32, wherein the siRNA inhibits or downregulates a gene whose upregulation is associated with immunosuppression of a cell.
34. The immunogenic construct of embodiment 33, wherein the cell is an antigen-presenting cell.
35. The immunogenic construct of embodiment 34, wherein the antigen-presenting cell is a dendritic cell or a macrophage.
36. The immunogenic construct of embodiment 35, wherein the gene is STAT3, IDO-1, IL-6, or PD-L1.
37. The immunogenic construct of any one of embodiments 29-35, wherein the at least one type of oligonucleotide is present at 1-10 wt. % of the nanoparticle.
38. The immunogenic construct of any one of embodiments 1-37, wherein the immunogenic construct further includes a targeting agent for a cell.
39. The immunogenic construct of embodiment 38, wherein the cell is an antigen-presenting cell.
40. The immunogenic construct of embodiment 39, wherein the antigen-presenting cell is a dendritic cell or a macrophage.
41. The immunogenic construct of embodiment 39 or 40, wherein the targeting agent is mannose, a monoclonal or polyclonal antibody or a fragment thereof that recognizes and binds to an epitope displayed on the antigen-presenting cell, or a ligand which binds to a surface receptor on the antigen-presenting cell.
42. The immunogenic construct of any one of embodiments 38-41, wherein the targeting agent is present at 0.1 to 10 wt. % of the nanoparticle.
43. The immunogenic construct of any one of embodiments 1-42, wherein the immunogenic construct further includes a labeling agent.
44. The immunogenic construct of embodiment 43, wherein the labeling agent is a fluorescent dye and/or a metal probe.
45. The immunogenic construct of any one of embodiments 1-44, having a hydrodynamic diameter of about 10 nm to about 10 microns.
46. The immunogenic construct of embodiment 45, having a hydrodynamic diameter of about 90 nm to about 150 nm.
47. The immunogenic construct of any one of embodiments 1-46, wherein the nanoparticle has a diameter of about 5 to about 999 nm.
48. An immunogenic construct including: a nanoparticle; a lipid layer coating an external surface of the nanoparticle; and an antigen or an antigen producing agent for an infectious agent.
49. The immunogenic construct of embodiment 40, further including an adjuvant.
50. The immunogenic construct of embodiment 49, wherein the adjuvant includes one or more of a CpG oligonucleotide, a DNA TLR agonist containing a CpG sequence, a non-CpG DNA TLR agonist, an RNA TLR agonist, an aluminum salt, an anti-CD40 antibody, a fusion protein, a cytokine, a small molecule TLR agonist, an oil- or surfactant-based adjuvant, a lipopolysaccharide, a plant extract, or a derivative thereof.
51. The immunogenic construct of embodiment 49 or 50, wherein the adjuvant includes a CpG oligonucleotide.
52. The immunogenic construct of any one of embodiments 49-51, wherein the adjuvant is loaded into the nanoparticle.
53. The immunogenic construct of any one of embodiments 49-52, wherein the adjuvant is loaded on or within the lipid layer.
54. The immunogenic construct of any one of embodiments 49-53, wherein the adjuvant is present at 1-20 wt. % of the nanoparticle.
55. The immunogenic construct of any one of embodiments 48-54, wherein the nanoparticle is a silica nanoparticle, a silicon nanoparticle, an iron oxide nanoparticle, a gold nanoparticle, a silver nanoparticle, a carbon nanotube, or an adjuvant nanoparticle.
56. The immunogenic construct of embodiment 55, wherein the nanoparticle is a calcium phosphate nanoparticle.
57. The immunogenic construct of any one of embodiments 48-56, wherein the lipid layer is a monolayer or multilayer membrane including one or more of lipids selected from a neutral lipid, a fatty-acid-modified lipid, a phospholipid, a fatty acid, a polymerizable lipid, a cationic lipid, a sphingolipid, and a sterol.
58. The immunogenic construct of embodiment 57, wherein the neutral lipid is a prostaglandin, an eicosanoid, or a glyceride; the fatty-acid-modified lipid is 1,2-diphytanoyl-sn-glycero-3-phosphocholine or 1-(12-biotinyl(aminododecanoyl))-2-oleoyl-sn-glycero-3-phosphoethanolamine; the phospholipid is phosphatidylcholine, phosphatidylethanolamine, 1,2-disteraoyl-sn-glycero-3-phosphochline, or 1,2-dioleoyl-sn-glycero-3-phosphoethanolamine; the fatty acid is stearic acid or lauric acid; the polymerizable lipid is cholesterol-PEG or distearoyl-rac-glycerol-PEG2K; the cationic lipid Is 1,2-dioleoyl-3-trimethylammonium-propane or dimethyldioctadecylammonium bromide; the sphingolipid is a sphingomyelin or a ceramide; and the sterol is a cholesterol or a stigmasterol.
59. The immunogenic construct of embodiment 58, wherein the lipid layer includes 1,2-dioleoyl-sn-glycero-3-phosphoethanolamine, dimethyldioctadecylammonium bromide, cholesterol, 1,2-distearoyl-sn-glycero-3-phosphocholine, and distearoyl-rac-glycerol-PEG2K.
60. The immunogenic construct of any one of embodiments 48-59, wherein the lipid layer is present at 0.1-99.9 wt. % of the nanoparticle.
61. The immunogenic construct of any one of embodiments 48-60, wherein the infectious agent is a virus.
62. The immunogenic construct of any one of embodiments 48-61, wherein the infectious agent is a beta-coronavirus.
63. The immunogenic construct of any one of embodiments 48-62, wherein the infectious agent is SARS-CoV-2, a SARS-CoV-1, or MERS-CoV.
64. The immunogenic construct of any one of embodiments 48-63, wherein the infectious agent is SARS-CoV-2.
65. The immunogenic construct of embodiment 64, wherein the antigen or antigen producing agent is a recombinant full-length protein.
66. The immunogenic construct of embodiment 65 wherein the antigen or antigen producing agent is a full-length SARS-CoV-2 spike glycoprotein, a SARS-CoV-2 nucleocapsid protein, or a SARS-CoV-2 membrane protein.
67. The immunogenic construct of embodiment 64, wherein the antigen or antigen producing agent is a protein subunit.
68. The immunogenic construct of embodiment 67, wherein the antigen or antigen producing agent is a protein subunit corresponding to the S1, S2, or RBD region of the SARS-CoV-2 spike glycoprotein.
69. The immunogenic construct of embodiment 64, wherein the antigen or antigen producing agent is a peptide corresponding to an immunogenic sequence of SARS-CoV-2 spike glycoprotein.
70. The immunogenic construct of embodiment 69, wherein the antigen or antigen producing agent has the peptide sequence any one of SEQ ID NOs: 1-8.
71. The immunogenic construct of embodiment 64, wherein the antigen or antigen producing agent is a mRNA or a pDNA.
72. The immunogenic construct of any one of embodiments 48-71, wherein the antigen or antigen producing agent is loaded into the nanoparticle.
73. The immunogenic construct of any one of embodiments 48-72, wherein the antigen or antigen producing agent is loaded on or within the lipid layer.
74. The immunogenic construct of any one of embodiments 48-73, wherein the antigen or antigen producing agent is present at 0.01 to 10 wt. % of the nanoparticle.
75. The immunogenic construct of any one of embodiments 48-74, wherein the immunogenic construct further includes at least one type of oligonucleotide.
76. The immunogenic construct of embodiment 75, wherein the at least one type of oligonucleotide includes a siRNA.
77. The immunogenic construct of embodiment 76, wherein the siRNA inhibits or downregulates a gene whose upregulation is associated with immunosuppression of a cell.
78. The immunogenic construct of embodiment 77, wherein the cell is an antigen-presenting cell.
79. The immunogenic construct of embodiment 78, wherein the antigen-presenting cell is a dendritic cell or a macrophage.
80. The immunogenic construct of embodiment 79, wherein the gene is STAT3, IDO-1, IL-6, or PD-L1.
81. The immunogenic construct of any one of embodiments 74-80, wherein the at least one type of oligonucleotide is present at 0.01 to 10 wt. % of the nanoparticle.
82. The immunogenic construct of any one of embodiments 48-81, wherein the immunogenic construct further includes a targeting agent for a cell.
83. The immunogenic construct of embodiment 82, wherein the cell is an antigen-presenting cell.
84. The immunogenic construct of embodiment 83, wherein the antigen-presenting cell is a dendritic cell or a macrophage.
85. The immunogenic construct of embodiment 84, wherein the targeting agent is mannose, a monoclonal or polyclonal antibody or a fragment thereof that recognizes and binds to an epitope displayed on the antigen-presenting cell, or a ligand which binds to a surface receptor on the antigen-presenting cell,
86. The immunogenic construct of any one of embodiments 48-85, wherein the immunogenic construct further includes a labeling agent.
87. The immunogenic construct of embodiment 86, wherein the labeling agent is a fluorescent dye and/or a metal probe.
88. The immunogenic construct of any one of embodiments 48-87, having a hydrodynamic diameter of about 10 nm to about 10 microns.
89. The immunogenic construct of embodiment 88, having a hydrodynamic diameter of about 90 nm to about 150 nm.
90. The immunogenic construct of any one of embodiments 1-89, wherein the nanoparticle has a size of about 5 nm to 999 nm.
91. A pharmaceutical composition including an immunogenic construct of any one of embodiments 1-90 and a pharmaceutically acceptable excipient.
92. A vaccine including an immunogenic construct of any one of embodiments 1-90 and a pharmaceutically acceptable excipient.
93. A method of co-delivering an antigen and an adjuvant to a cell including contacting the cell with an immunogenic construct of any one of embodiments 1-90.
94. The method of embodiment 93, wherein the cell is an antigen-presenting cell.
95. The method of embodiment 94, wherein the cell is a dendritic cell or a macrophage.
96. The method of embodiment 93, wherein the cell is a muscle cell.
97. A method of inducing an immune response against an infectious agent in a subject including administering to the subject an immunogenic amount of an immunogenic construct of any one of embodiments 1-90.
98. The method of embodiment 97, wherein the subject is a human.
99. The method of embodiment 97 or 98, wherein the subject is immunocompromised.
100. The method of any one of embodiments 97-99, wherein the immunogenic construct is administered by intramuscular injection.
101. A method of treating or preventing an infectious disease in a subject including administering to the subject an immunogenic amount of an immunogenic construct of any one of embodiments 1-90.
102. The method of embodiment 101, wherein the subject is a human.
103. The method of embodiment 101 or 102, wherein the subject is immunocompromised.
104. The method of any one of embodiments 101-103, wherein the immunogenic construct is administered intramuscularly, by inhalation, or intranasally.

### Exemplary Embodiments Set 2

1. An immunogenic construct including: a nanoparticle platform (NP), including: a nanoparticle; an amount of crosslinked cationic polymer including polyethylenimine (PEI) bound electrostatically to an exterior surface of the nanoparticle, and wherein the PEI content is at least 10% by weight of the NP; and an amount of a stabilizer including polyethylene glycol (PEG) bound covalently to the crosslinked PEI; and an antigen, or an antigen producing agent, of an infectious agent, wherein the hydrodynamic size of the construct is no more than 1 micron.
2. The immunogenic construct of embodiment 1, wherein the nanoparticle is a mesoporous silica nanoparticle (MSNP).
3. An immunogenic construct including: a nanoparticle platform (NP), including: a nanoparticle; a crosslinked cationic polymer bound to an exterior surface of the nanoparticle; and a stabilizer bound to the crosslinked cationic polymer or the exterior surface of the nanoparticle; and an antigen, or an antigen producing agent, of an infectious agent.
4. The immunogenic construct of embodiment 1 or embodiment 3, further including an adjuvant.
5. The immunogenic construct of embodiment 4, wherein the adjuvant includes one or more of a CpG oligonucleotide, a DNA TLR agonist containing a CpG sequence, a non-CpG DNA TLR agonist, an RNA TLR agonist, an aluminum salt, an anti-CD40 antibody, a fusion protein, a cytokine, a small molecule TLR agonist, an oil- or surfactant-based adjuvant, a lipopolysaccharide, a plant extract, or a derivative thereof.
6. The immunogenic construct of embodiment 5, wherein the adjuvant includes a CpG oligonucleotide.
7. The immunogenic construct of embodiment 4, wherein the adjuvant includes poly I:C.
8. The immunogenic construct of any one of embodiments 4-7, wherein the adjuvant is present at 1-20 wt. % of the NP.
9. The immunogenic construct of embodiment 1 or embodiment 3, or any other embodiment in this set, wherein the nanoparticle is a silica nanoparticle, a silicon nanoparticle, an iron oxide nanoparticle, a gold nanoparticle, a silver nanoparticle, a calcium carbonate nanoparticle, a calcium phosphate nanoparticle, a carbon nanotube, or an adjuvant nanoparticle.
10. The immunogenic construct of embodiment 9, wherein the nanoparticle is a mesoporous silica nanoparticle (MSNP).
11. The immunogenic construct of embodiment 10, wherein MSNP has an average pore size of 2-6 nm, 7 nm, or less than 7 nm.
12. The immunogenic construct of embodiment 9, wherein the nanoparticle is an iron oxide nanoparticle.
13. The immunogenic construct of embodiment 1 or embodiment 3, or any other embodiment in this set, wherein the cationic polymer includes PEI, chitosan, polypropyleneimine, polylysine, polyamidoamine, poly(allylamine), poly(diallyldimethylammonium chloride), poly(N-isopropyl acrylamide-co-acrylamide), poly(N-isopropyl acrylamide-co-acrylic acid), diethylaminoethyl-dextran, poly-(N-ethyl-vinylpyridinium bromide), poly(dimethylamino)ethyl methacrylate, poly(ethylene glycol)-co-poly(trimethylaminoethylmethacrylate chloride), or a mixture of two or more thereof.
14. The immunogenic construct of embodiment 1 or embodiment 3, wherein the cationic polymer is or includes PEI.
15. The immunogenic construct of embodiment 1 or embodiment 3, or any other embodiment in this set, wherein the cationic polymer has a molecular weight of about 0.8 kDa to about 25 kDa.
16. The immunogenic construct of embodiment 1 or embodiment 3, or any other embodiment in this set, wherein the cationic polymer is present at 1-50 wt. % of the NP.
17. The immunogenic construct of embodiment 1 or embodiment 3, or any other embodiment in this set, wherein the stabilizer includes PEG, dextran, polysialic acid, hyaluronic acid, polyvinyl pyrrolidone, polyvinyl alcohol, polyacrylamide, or a mixture of two or more thereof.
18. The immunogenic construct of embodiment 17, wherein the stabilizer is PEG.
19. The immunogenic construct of embodiment 1 or embodiment 3, or any other embodiment in this set, wherein the stabilizer has a molecular weight of about 1 kDa to about 20 kDa, or about 5 kDa.
20. The immunogenic construct of embodiment 1 or embodiment 3, or any other embodiment in this set, wherein the stabilizer is present at 1-50 wt. %, about 10-30 wt. %, about 5 to 20 wt. %, about 15 wt. %, or about 20 wt. % of the NP.
21. The immunogenic construct of embodiment 1 or embodiment 3, or any other embodiment in this set, wherein the antigen includes a protein, and the protein antigen is conjugated onto the stabilizer.
22. The immunogenic construct of embodiment 1 or embodiment 3, wherein the antigen is a peptide, and the peptide antigen is bound electrostatically to the crosslinked cationic polymer.
23. The immunogenic construct of embodiment 1 or embodiment 3, or any other embodiment in this set, wherein the antigen producing agent is a mRNA or a pDNA, and the antigen producing agent is bound electrostatically to the crosslinked cationic polymer.
24. The immunogenic construct of embodiment 1 or embodiment 3, or any other embodiment in this set, wherein the infectious agent is a virus.
25. The immunogenic construct of embodiment 24, wherein the infectious agent is a beta-coronavirus.
26. The immunogenic construct of embodiment 25, wherein the infectious agent is SARS-CoV-2, a SARS-CoV-1, or MERS-CoV.
27. The immunogenic construct of embodiment 26, wherein the infectious agent is SARS-CoV-2.
28. The immunogenic construct of embodiment 27, wherein the antigen is, or the antigen producing agent encodes, a recombinant full-length SARS-CoV-2 protein.
29. The immunogenic construct of embodiment 21, or any other embodiment in this set, wherein the full-length SARS-CoV-2 protein is a SARS-CoV-2 spike glycoprotein, a SARS-CoV-2 nucleocapsid protein, or a SARS-CoV-2 membrane protein.
30. The immunogenic construct of embodiment 27, wherein the antigen is, or the antigen producing agent encodes, a protein subunit.
31. The immunogenic construct of embodiment 30, wherein the protein subunit corresponds to SARS-CoV-2 spike glycoprotein S1 region, S2 region, or Receptor Binding Domain (RBD) region.
32. The immunogenic construct of embodiment 27, or any other embodiment in this set, wherein the antigen is, or the antigen producing agent encodes, a peptide corresponding to an immunogenic sequence of SARS-CoV-2 spike glycoprotein.
33. The immunogenic construct of embodiment 32, wherein the peptide includes the sequence of any one of SEQ ID NOs: 1-8.
34. The immunogenic construct of embodiment 27, wherein the antigen producing agent is a mRNA or a pDNA.
35. The immunogenic construct of embodiment 1 or embodiment 3, or any other embodiment in this set, wherein the infectious agent is a bacterium, a parasite, a protozoan, or a fungus.
36. The immunogenic construct of embodiment 1 or embodiment 3, or any other embodiment in this set, wherein the antigen or antigen producing agent is present at 0.5-20 wt. % of the NP.
37. The immunogenic construct of embodiment 1 or embodiment 3, wherein the immunogenic construct further includes at least one oligonucleotide
38. The immunogenic construct of embodiment 37, wherein the at least oligonucleotide is electrostatically bound to the cationic polymer.
39. The immunogenic construct of embodiment 38, wherein the at least one oligonucleotide includes a siRNA, a miRNA, a miRNA mimic, or an antisense oligonucleotide.
40. The immunogenic construct of embodiment 38, wherein the at least one oligonucleotide includes a siRNA.
41. The immunogenic construct of embodiment 40, wherein the siRNA inhibits or downregulates a gene the expression or upregulation of which is associated with immunosuppression of a cell.
42. The immunogenic construct of embodiment 41, wherein the cell is an antigen-presenting cell.
43. The immunogenic construct of embodiment 42, wherein the antigen-presenting cell is a dendritic cell or a macrophage.
44. The immunogenic construct of embodiment 43, wherein the gene is STAT3, IDO-1, IL-6, or PD-L1.
45. The immunogenic construct of embodiment 37, wherein the oligonucleotide is present at 1-10 wt. % of the NP.
46. The immunogenic construct of embodiment 1 or embodiment 3, or any other embodiment in this set, wherein the immunogenic construct further includes a targeting agent for a cell.
47. The immunogenic construct of embodiment 46, wherein the cell is an antigen-presenting cell.
48. The immunogenic construct of embodiment 47, wherein the antigen-presenting cell is a dendritic cell or a macrophage.
49. The immunogenic construct of 48, wherein the targeting agent includes at least one of mannose, a monoclonal or polyclonal antibody or a fragment thereof that recognizes and binds to an epitope displayed on the antigen-presenting cell, or a ligand that binds to a surface receptor on the antigen-presenting cell.
50. The immunogenic construct of embodiment 3, having a hydrodynamic diameter of about 10 nm to about 10 microns.
51. The immunogenic construct of embodiment 1 or embodiment 3, or any other embodiment in this set, having a hydrodynamic diameter of about 30 nm to about 200 nm.
52. The immunogenic construct of embodiment 1 or embodiment 3, or any other embodiment in this set, having a hydrodynamic diameter of about 80 nm to about 999 nm.
53. An immunogenic composition including a plurality of the immunogenic constructs of embodiment 1 or embodiment 3, or any other embodiment in this set.
54. A composition including: an immunogenic construct of embodiment 1 or embodiment 3, or any other embodiment in this set, and at least one biologically or pharmaceutically acceptable excipient.
55. A vaccine including: an immunogenic construct of embodiment 1 or embodiment 3, or any other embodiment in this set, and a pharmaceutically acceptable excipient.
56. A method of co-delivering an antigen and an adjuvant to a cell including: contacting the cell with an immunogenic construct of embodiment 1 or embodiment 3, or any other embodiment in this set.
57. The method of embodiment 56, wherein the cell is an antigen-presenting cell.
58. The method of embodiment 57, wherein the cell is a dendritic cell or a macrophage.
59. The method of embodiment 56, wherein the cell is a muscle cell.
60. A method including administering to a subject an immune-stimulatory amount of an immunogenic construct of embodiment 1 or embodiment 3, or any other embodiment in this set.
61. The method of embodiment 60, which results in inducing an immune response against an infectious agent in the subject.
62. The method of embodiment 60, which results in treating or preventing an infectious disease in the subject.
63. The method of embodiment 62, wherein the subject is a human.
64. The method of embodiment 62, wherein the subject is immunocompromised.
65. The method of embodiment 62, wherein the immunogenic construct is administered transdermally, intramuscularly, by inhalation, or intranasally.

### XIV. Experimental Examples

### Example 1. Preparation of Immunogenic Constructs

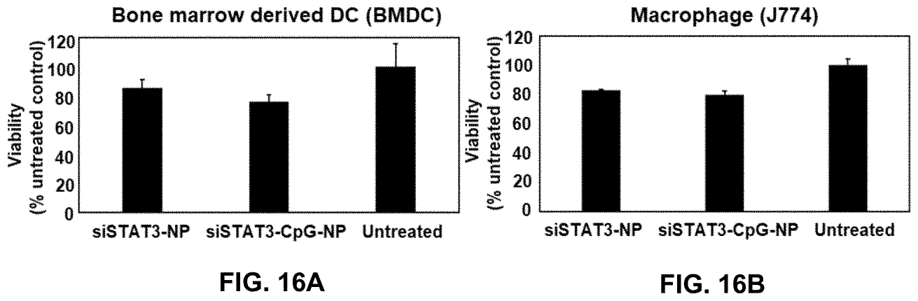

Mesoporous silica-based nanoparticles coated with PEI and PEG were synthesized as previously described (see, e.g., International Application No. PCT/US2016/022655, which is incorporated by reference herein in its entirety). Briefly, mesoporous silica nanoparticles (MSNP) were synthesized by sol-gel synthesis. The MSNP core was coated layer-by-layer with polyethylenimine (PEI) and polyethyleneglycol (PEG). PEI on the MSNP was also cross-linked (predominantly, to itself) for enhanced oligonucleotide delivery efficacy and safety (see Ngamcherdtrakul et al., Advanced Functional Materials, 25(18):2646-2659, 2015). Such crosslinking increases buffering capacity and endosomal escape of the cargos, and also reduces the surface charges of the nanoparticle platform (NP). Reducing surface charge of NP or immunogenic construct results in safety of antigen presenting cells, which are important for vaccination (see FIG. 16). The pore size of the MSNP used in this Example was measured by TEM to be 2-3 nm, and 6.6 nm by Barrett-Joyner-Halenda (BJH) pore size analysis (e.g., via nitrogen adsorption and desorption). MSNP coated with crosslinked PEI and PEG is referred to henceforth as a nanoparticle platform, or “NP”.

CpG 1826 (mouse sequence; Invivogen) was loaded electrostatically on the NP by 10-minute mixing, though a shorter time (2-5 minutes) was also effective. Loading was performed in a complete binding manner as confirmed by the absence of free cargo molecules in the supernatant upon separating out cargo-loaded NP by centrifugation. siRNA was conjugated with Dy677 dye (Dharmacon) and thus was quantified by fluorescence signal. The unbound CpG and siRNA cargo content in the supernatant was measured by Nanodrop spectrophotometer, microplate spectrophotometer, or gel electrophoresis. SIINFEKL (SF; SEQ ID NO: 90) peptide was loaded via non-covalent interaction with the NP (on the PEI layer) by 2-hour mixing with NP, though a shorter time was also effective. Large proteins (antibody, full length proteins) are typically loaded by covalent bonding (Example 4), but non-covalent bonding is also possible. Large protein cargos, such as spike proteins, are not encapsulated inside the small pores (e.g., 2-6 nm) of mesoporous silica, but instead are attached to the surface of the materials (e.g., conjugated, or electrostatically bound on the PEG-PEI layer, or adsorption onto an external silica surface).

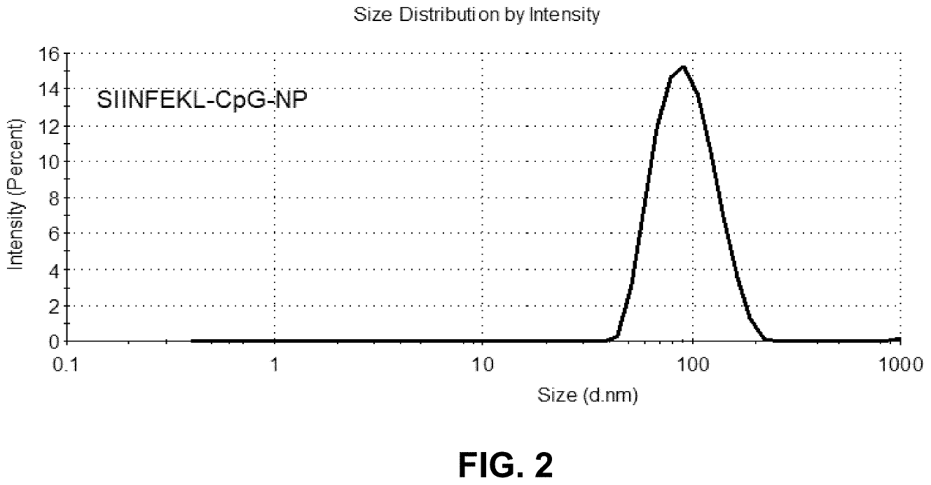

The amount of unbound peptide or protein in the supernatant (upon centrifuge) was characterized by either fluorescent signal of the fluorescent dye conjugated on peptide/protein or BCA assay. Other protein assays can also be used. With multiple types of cargo loading, the hydrodynamic sizes (diameters) of the resulting immunogenic constructs remain under five microns (for instance under one micron), suitable for cellular uptake. As shown in FIG. 2, when the NPs were loaded with 5 wt. % SF (via non-covalent binding) and 2 wt. % CpG, they maintained the hydrodynamic size of about 100 nm. As shown in Table 3, nanoparticle platforms (MSNP-PEI-PEG [MSNP loaded with 15 wt. % crosslinked PEI and 10 wt. % PEG] characterized by thermogravimetric analysis (TGA)) were loaded with about 3 wt. % spike protein (e.g., via covalent bonding), 2 wt. % siRNA, and 4 wt. % CpG and maintained a hydrodynamic size of less than 150 nm. siRNA and CpG are loaded last on the immunogenic construct. With about 2-4 wt. % of siRNA loading, CpG can be loaded from about 4-9 wt. % while maintaining the size below 150 nm (Table 4). CpG, siRNA, and peptides were loaded by mixing with NP solution as described prior in a complete binding manner (confirmed by the absence of unbound cargo in the supernatant upon centrifuge) at the corresponding final wt. %. CpG 1826 (mouse sequence) was used throughout Examples 1-4, but CpG 7909/2006 (human sequence) was also evaluated and yielded similar characteristics. All percent loading is by weight of NP.

Table 3 shows the hydrodynamic sizes of 1) mesoporous silica nanoparticles coated with 15 wt. % crosslinked PEI and 10 wt. % PEG (NP), 2) NP loaded with about 2 wt. % siRNA and about 6 wt. % CpG, 3) spike protein (3 wt. %) conjugated NP (Spike-NP), and 4) spike-NP loaded with about 2 wt. % siRNA and about 4 wt. % CpG. Average size (Z-average) and polydispersity index (PDI) is shown from three measurements using a Malvern Zetasizer. All loadings are by weight of the nanoparticle platform (NP or PEG-PEI-MSNP).

TABLE 3

| Material | Hydrodynamic size | PDI |
|---|---|---|
| NP | 90.1 ± 0.8 | 0.17 ± 0.03 |
| (2%) siRNA-(6%) CpG-NP | 106.6 ± 0.4 | 0.15 ± 0.01 |
| Spike-NP | 84.4 ± 0.5 | 0.12 ± 0.01 |
| Spike-(2%) siRNA-(4%) CpG-NP | 146.1 ± 6.1 | 0.18 ± 0.01 |

Table 4 shows the hydrodynamic sizes of mesoporous silica nanoparticles coated with PEI and PEG (NP) and loaded with about 2-4 wt. % siRNA and about 4-9 wt. % CpG by weight of the NP.

TABLE 4

| Material | Hydrodynamic size | PDI |
|---|---|---|
| (2%) siRNA-(4%) CpG-NP | 95.5 ± 1.3 | 0.14 ± 0.01 |
| (2%) siRNA-(6%) CpG-NP | 106.6 ± 0.4 | 0.15 ± 0.01 |
| (2%) siRNA-(9%) CpG-NP | 146.7 ± 1.5 | 0.22 ± 0.03 |
| (4%) siRNA-(6%) CpG-NP | 120.4 ± 1.7 | 0.17 ± 0.02 |

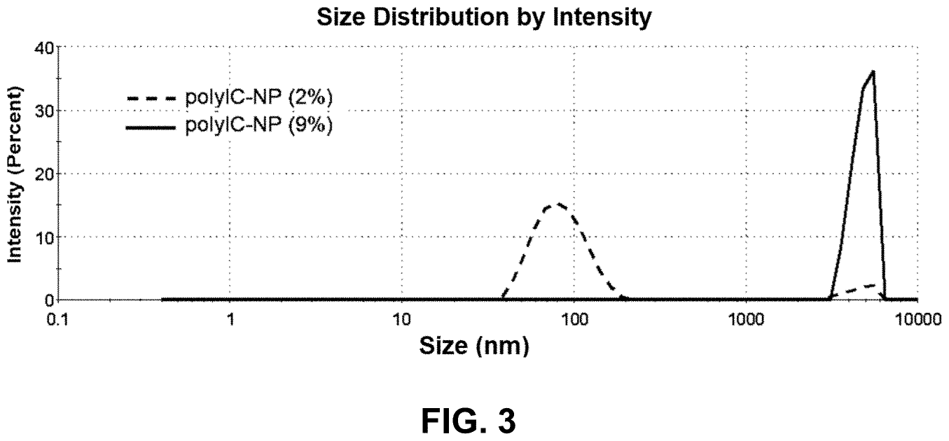

In addition to CpG, other adjuvants such as Poly I:C (Adipogen) can be loaded non-covalently on the immunogenic construct. Poly I:C was loaded on the NP electrostatically and in a complete binding manner by 10-minute mixing of Poly I:C solution and NP solution. The hydrodynamic size remained small (88 nm) at about 2 wt. % loading but increased significantly (4.8 micron) at about 9 wt. % loading (FIG. 3).

### Example 2. NP Co-Delivery of CpG and Oligonucleotide (e.g., siRNA) to Knockdown Immunosuppressive Genes (e.g., STAT3, PD-L1)

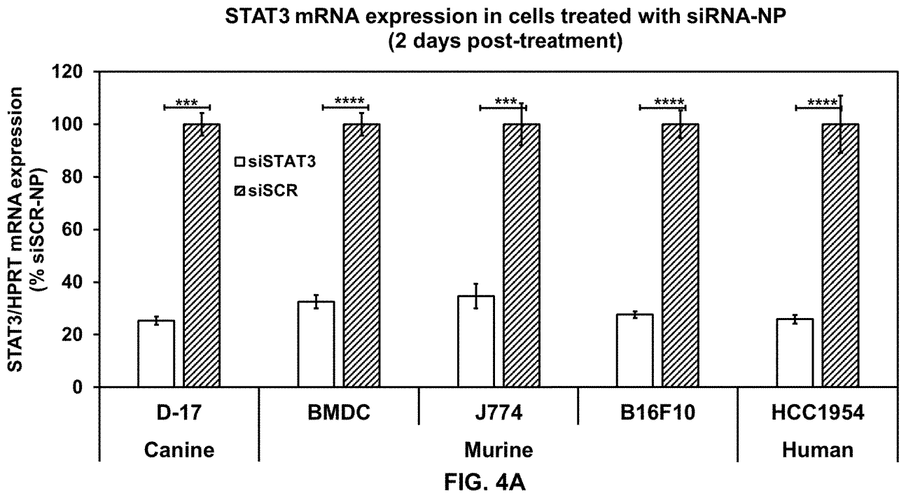

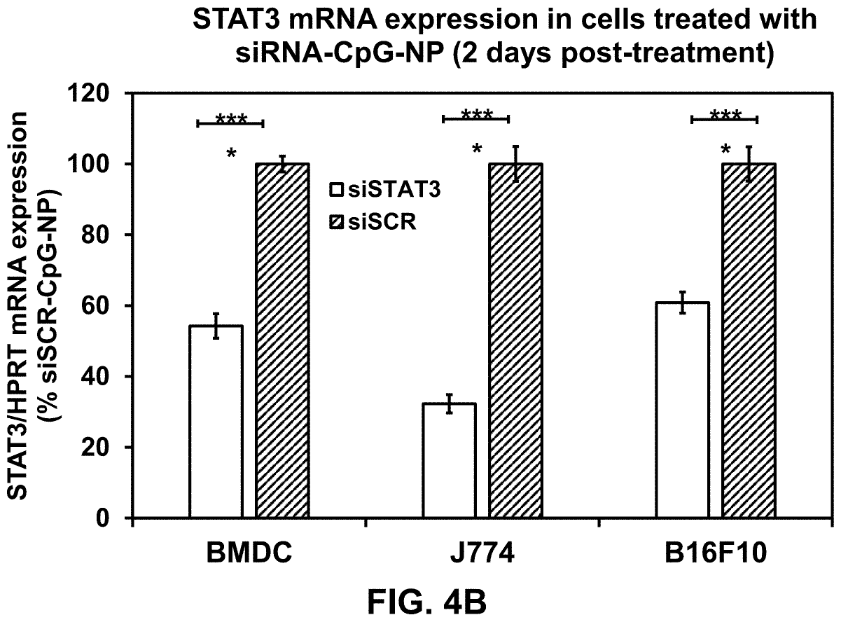

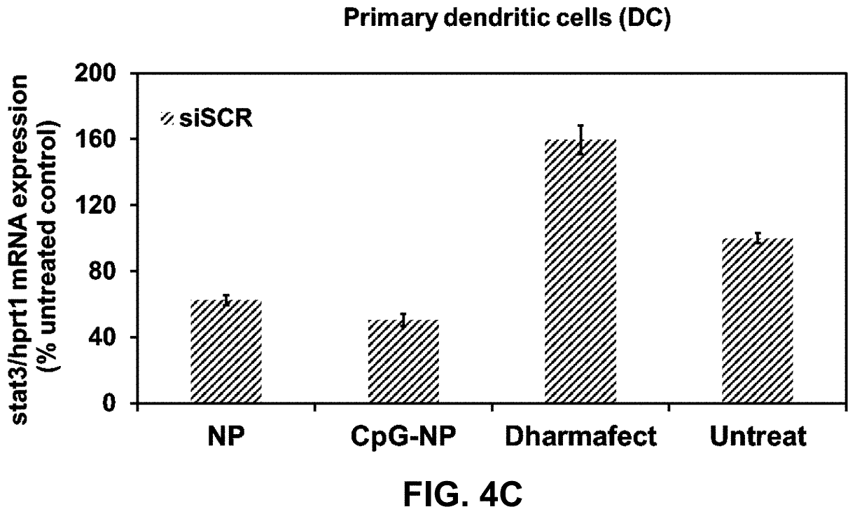

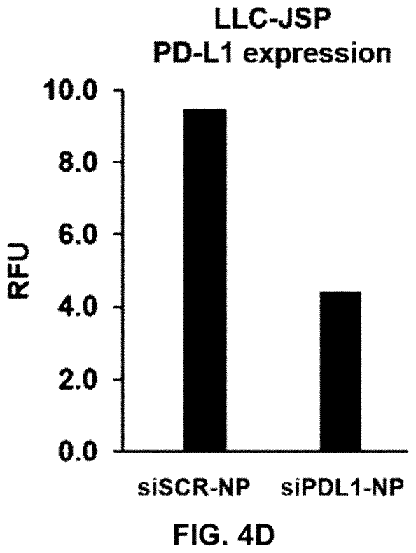

STAT3 is considered a strong immunosuppressive gene. To determine the knockdown efficacy of NP loaded with siRNA against STAT3 (siSTAT3), immune cells (i.e., dendritic cells, macrophages) and various cancer cells (B16-F10, HCC1954, D17) were treated with siSTAT3-NP (mesoporous silica coated with PEI and PEG, from Example 1) for 48 hours before RNA isolation and qRT-PCR to determine the knockdown efficacy of STAT3 in the cell lines. As shown in FIG. 4A, the immunogenic constructs (siSTAT3-NP) results in >70% target gene knockdown in both immune and cancer cell lines. Same siSTAT3 sequence can knock down STAT3 gene in human, canine, and mouse cells (FIG. 4A). The same immunogenic construct loaded with both siRNA and CpG was still effective at knocking down STAT3 in both immune and cancer cells (FIG. 4B). Interestingly, it was found that siSCR-NP also decreased STAT3 level (see FIG. 4C) in DCs. This was not caused by NP toxicity since the cell viability was unchanged versus untreated control, and STAT3 mRNA was normalized with housekeeping mRNA. Without being bound to any explanation, it is proposed that this may be due to the antioxidant property of mesoporous silica nanoparticle because antioxidants were previously reported to counteract immunosuppressive pathways, including STAT3 activation (Yoon et al., Autophagy, 6(8):1125-1138, 2010). On the other hand, it was found that Dharmafect (a commercial transfection agent based on cationic lipid (non-antioxidant) by Horizon Discovery) increased STAT3 expression in DCs (FIG. 4C), which may lead to undesirable immunosuppressive TME. FIG. 4D shows that the nanoparticle can also be used to deliver PD-L1 siRNA to knock down PD-L1 protein expression in lung cells. This suggests that the use of the antioxidant mesoporous silica nanoparticle platform described in Ngamcherdtrakul et al., Advanced Functional Materials, 25(18):2646-2659, 2015 and Morry et al., Biomaterials, 66:41-52, 2015 may be advantageous over lipid nanoparticles for controlling STAT3-mediated pathways.

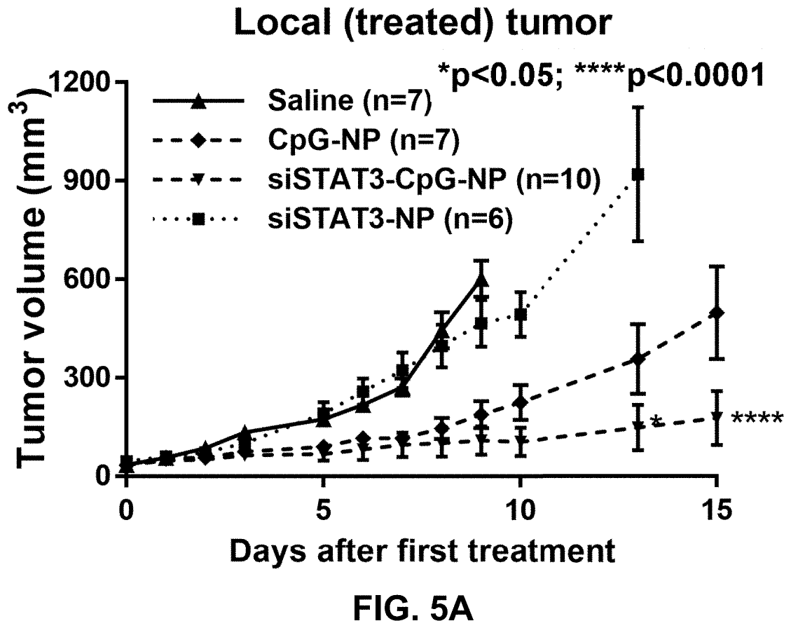

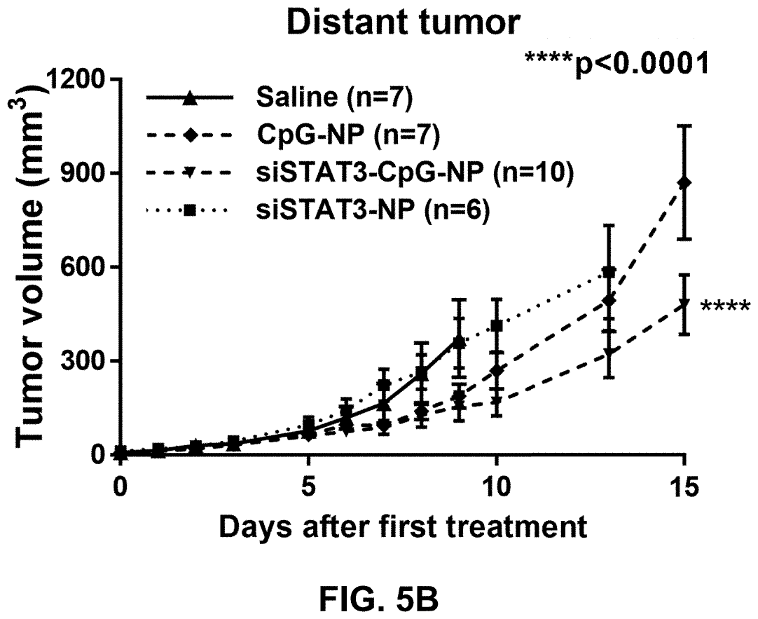

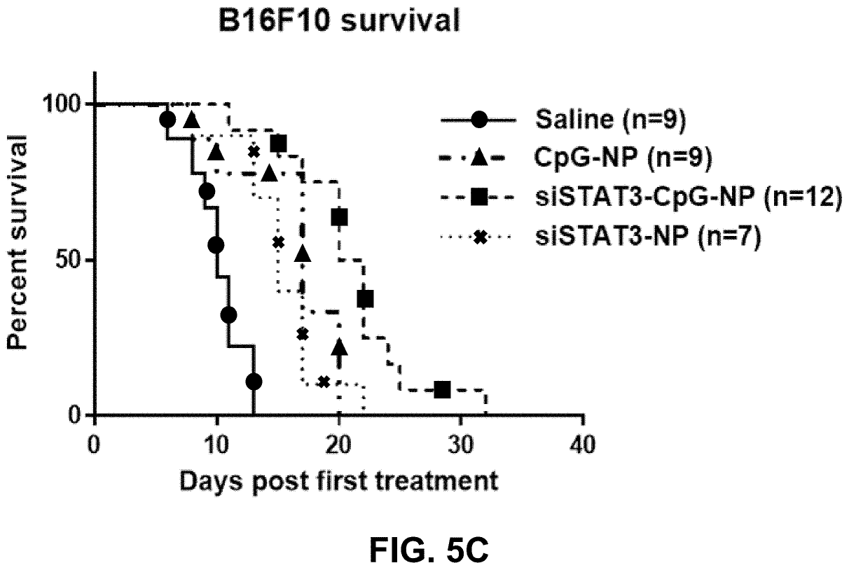

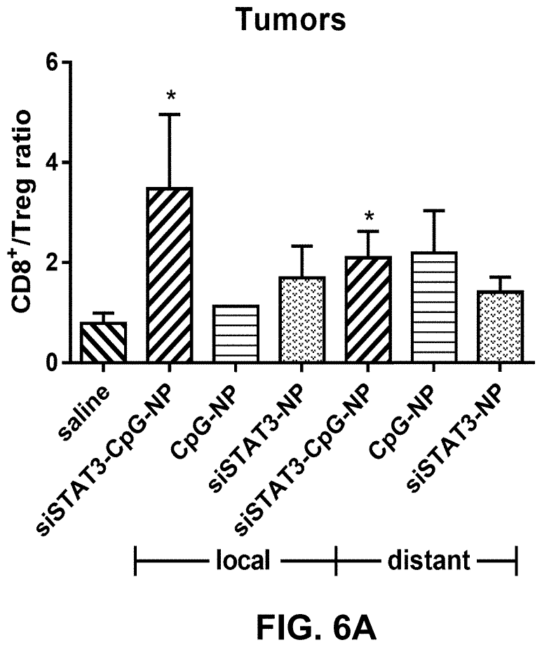

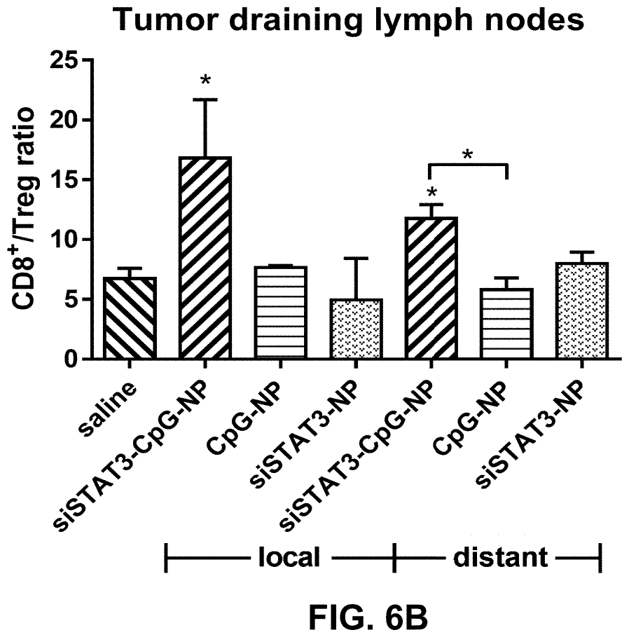

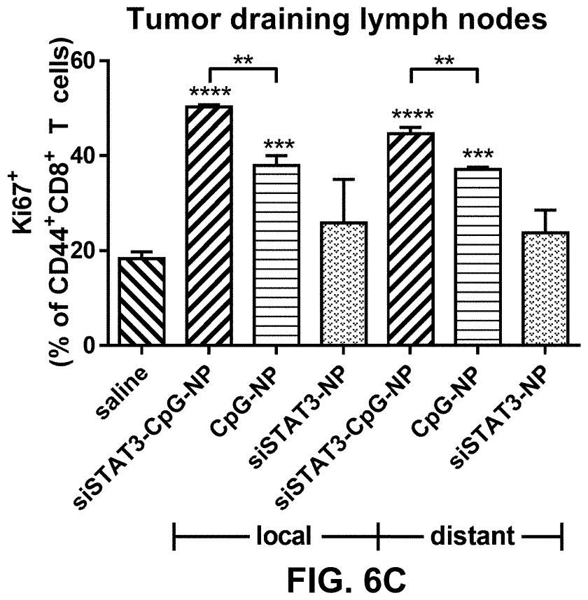

NP (PEG-PEI-MSNP) can be loaded with 2-4 wt. % siRNA and 4-9 wt. % of NP while maintaining the hydrodynamic size of the immunogenic construct under 150 nm (Table 4). In vivo NP codelivery of CpG and siSTAT3 triggers adaptive immunity better than NP delivery of CpG or siSTAT3 alone as shown in FIGS. 5 and 6. C57BU6 mice were implanted with 250K and 100K B16F10 melanoma cells on both shoulders to model local (primary) and distal (metastatic) tumors, respectively. Eight days after tumor implantation, treatments were intratumorally injected into the local tumor for a total of three doses, each at three days apart, while the distant tumor was left untreated. Tumor sizes and survival were monitored at least twice weekly as shown in FIGS. 5A-C. FIG. 5 shows that NP co-delivery of siSTAT3 and CpG (siSTAT3-CpG-NP) mudulates immunosuppressive environment (by siSTAT3), leading to greater whole-body anti-tumor immune response, evidenced by greater tumor reduction in both local treated tumors (FIG. 5A) and distal tumors (FIG. 5B), and greater surivival (FIG. 5C) compared to NP delivering CpG (CpG-NP) or siTAT3 alone (siSTAT3-NP). FIG. 6 shows that siSTAT3-CpG-NP treatment resulted in significantly higher CD8/Treg ratios in both local (treated) and distal (untreated) tumors (FIG. 6A) and associated draining lymph nodes (DLNs) (p<0.05 vs. saline) (FIG. 6B), confirming successful in situ tumor vaccination. Regulatory T cells (Treg) are typically elevated in patients' tumors and suppress anti-tumor immune response, including CD8⁺ T cell activity. The activated T cells also more proliferated in tumor draining lymph nodes (FIG. 6C). These results demonstrate the ability of the immunogenic construct to knockdown a target gene in immune cells and other cells (e.g., cancer or muscular cells), and provide rationale for using the immunogenic constructs of the disclosure for the co-delivery of siRNA and adjuvant (e.g., CpG). Note that for in situ cancer vaccination in FIGS. 5 and 6, tumors were relied upon as the depot for antigens (hence no external antigens were added to the constructs).

### Example 3. NP Co-Delivery of CpG and Antigen to Induce Adaptive (Antigen-Specific) Immune Response

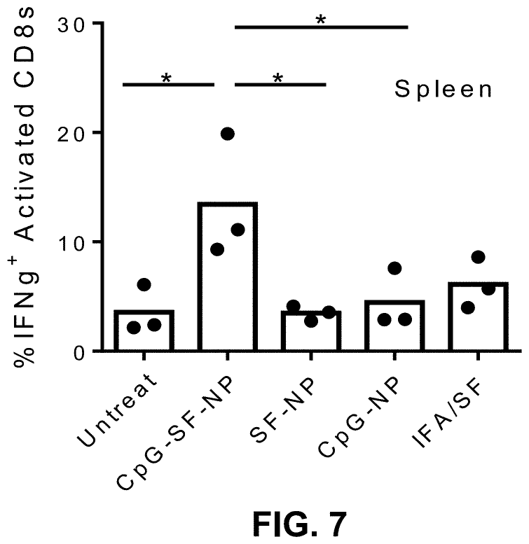

A study was conducted to determine the ability of CpG-SF-NP (NP loaded with CpG and SF) to induce systemic antigen-specific immunity. Briefly, C57BL/6 mice (n=3/group) were injected via footpad with the immunogenic constructs of CpG-SF-NP, NP loaded with SF (SF-NP) or CpG alone (CpG-NP), and SF formulated with Incomplete Freund's Adjuvant (IFA) (IFA/SF). Untreated mice were included as a control. One week after treatment, cells were harvested from the spleen of the mice, which were treated with Golgiblock and incubated in the presence or absence of SF for 6 hours. Intracellular IFNγ, the production of which corresponds to CD8+ T-cell response, was analyzed by flow cytometry. As shown in FIG. 7, CpG-SF-NP induced superior CD8+ T-cell response following ex vivo peptide re-stimulation. The results from Examples 1-3 provide strong rationale for using immunogenic constructs of the disclosure for co-delivery of adjuvant, antigen, and/or oligonucleotide.

### Example 4. Synthesis of Spike Protein-Conjugated NP (Immunogenic Constructs)

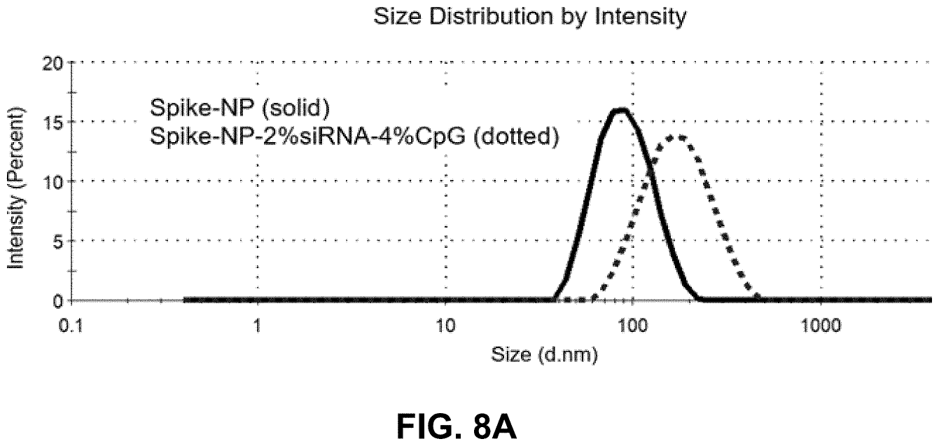

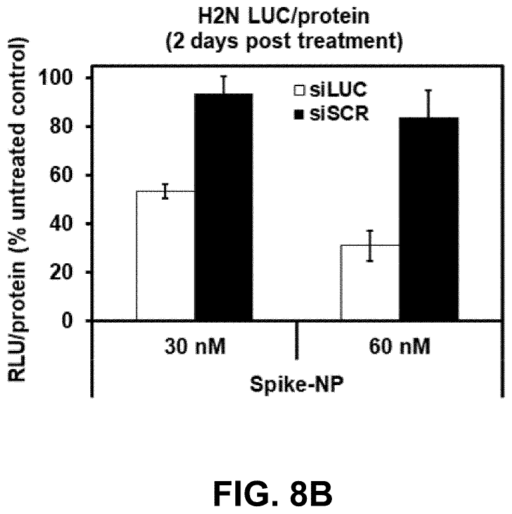

As an example, the full-length SARS-CoV-2 spike glycoprotein (Sino Biological) was covalently attached to the NP (PEI-PEG-MSNP). Briefly, the spike protein was thiolated for 1 hour prior to mixing with the NP and shaking overnight at 4° C. After shaking, the final immunogenic construct was washed, and final spike protein content was determined to be 3 wt. % of the NP based on the amount of unbound protein as determined by a BCA assay of the supernatant. siRNA (against luciferase or non-target scrambled siRNA) and CpG were loaded last on the immunogenic construct via electrostatic interactions (shaking for 5-10 minutes at room temperature). The hydrodynamic size of the spike-conjugated NP was less than 100 nm and remained smaller than 150 nm after loading of the spike glycoprotein (Table 3 and FIG. 8A). Spike-NP could effectively deliver siRNA to knock down a model gene (luciferase) in human cells (FIG. 8B). In brief, cells were plated at 3500 cells per well and incubated overnight at 37° C. Next day, cells were treated with spike-NP at siRNA dose of 30 or 60 nM. 48 h post treatment, cells were lysed and analyzed for luciferase activity by luciferase glow assay kit (ThermoFisher Scientific) and protein concentration by BCA protein assay kit following manufacturing protocols (ThermoFisher Scientific). Luciferase activity of the lysate was normalized with the corresponding protein concentration in the same well and reported as the percentage of the untreated control. All treatments were performed in quadruplicates.

### Example 5. Synthesis of Lipid-Coated Calcium Phosphate Nanoparticle Platform (CaP-L)

A calcium phosphate nanoparticle (CaPNP) was synthesized in a water-in-oil micro emulsion. Briefly, 60 μL of 2.5 M CaCl₂) (Fisher Scientific, USA) was dispersed in 4 mL of cyclohexane (Sigma, USA)/Igepal CO-520 (Sigma, USA) (71:29, v/v) to form a calcium phase. 60 μL of 12.5 mM Na₂HPO₄ (Fisher Scientific, USA) was dispersed in another 4 mL of cyclohexane/Igepal CO-520 (71:29, v/v) to form a phosphate phase. 40 μL of 20 mM 1,2-dioleoyl-sn-glycero-3-phosphoethanolamine (Avanti Polar Lipids, USA) in CHCl₃ (Fisher Scientific, USA) were added to the phosphate phase. The phosphate phase was then added dropwise into the calcium phase. The mixture was stirred for 0, 10, 15, or 20 minutes at room temperature. An equal volume of ethanol (Decon Labs, USA) was added to break the microemulsion. The CaPNP was collected with centrifugation at 21,000 g for 15 minutes and then washed 3 times with absolute ethanol to remove residual oil phase. The pellets were suspended in 100 μL of CHCl₃. The CaP-L was formed by mixing 10 μL of CaPNP in CHCl₃ with 1.4 μL of 20 mM dimethyldioctadecylammonium bromide (Sigma, USA), 1.4 μL of 20 mM cholesterol (Sigma, USA), 2.8 of 20 mM distearoyl-rac-glycerol-PEG2K (Avanti Polar Lipids, USA), and 0.7 μL of 20 mM 1,2-distearoyl-sn-glycero-3-phosphocholine (Sigma, USA) in CHCl₃, following by a minor bath sonication. CHCl₃ was then removed under reduced pressure, and CaP-L was formed by rehydration with 100 μL of PBS 1× (pH=7.2). The hydrodynamic sizes of CaP-L were measured with Zetasizer (ZS-90/Malvern, Malvern, U.K.) (Table 5).

Table 5 shows the hydrodynamic sizes of lipid-coated calcium phosphate NP (CaP-L) and CaP-L loaded with siRNA at 0.3% by weight of CaP-L.

TABLE 5

| Material | Hydrodynamic size | PDI |
|---|---|---|
| CaP-L | 98.1 ± 0.6 | 0.25 ± 0.02 |
| siRNA/CaP-L | 90.6 ± 2.8 | 0.26 ± 0.03 |

Synthesis of siRNA Loaded Lipid-Coated CaP-L (siRNA-CaP-L)

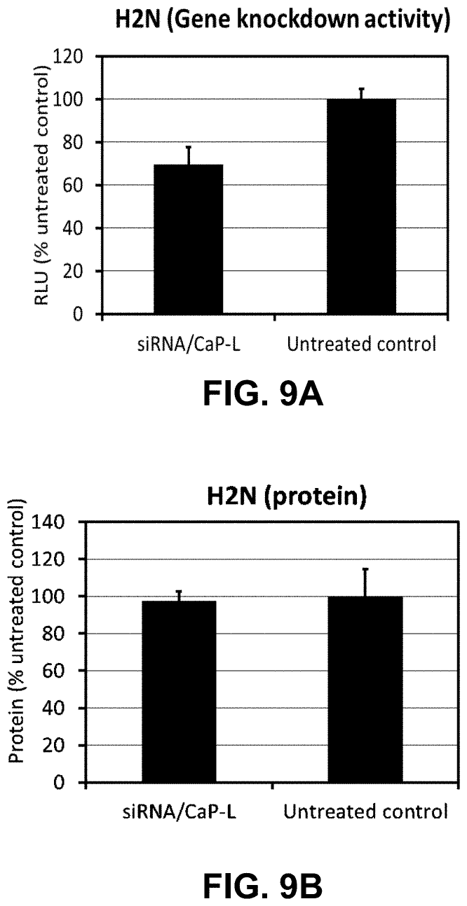

A siRNA loaded calcium phosphate nanoparticle was synthesized in a water-in-oil microemulsion. Briefly, 5 μg of siRNA was added to 60 μL of 2.5 M CaCl₂) (Fisher Scientific, USA), and the resulting solution was subsequently dispersed in 4 mL of cyclohexane/Igepal CO-520 (Sigma, USA) (71:29, v/v) to form a calcium phase. 60 μL of 12.5 mM Na₂HPO₄ (Fisher Scientific, USA) was dispersed in another 4 ml of cyclohexane/Igepal CO-520 (71:29, v/v) to form a phosphate phase. 40 μL of 20 mM 1,2-dioleoyl-sn-glycero-3-phosphoethanolamine (Avanti Polar Lipids, USA) in CHCl₃ (Fisher Scientific, USA) was added to the phosphate phase, then the phosphate phase was then added dropwise into the calcium phase. The mixture was stirred for 10 minutes at room temperature, after which an equal volume of ethanol (Decon Labs, USA) was added to break the microemulsion. The resulting siRNA-CaPNP was collected with centrifugation at 21,000 g for 15 minutes and then washed three times with absolute ethanol to remove the residual oil phase. The pellets thus obtained were suspended in 100 μL of CHCl₃. siRNA-CaP-L was formed by mixing 10 μl of siRNA-CaPNP in CHCl₃ with 1.4 μl of 20 mM dimethyldioctadecylammonium bromide (Sigma, USA), 1.4 μL of 20 mM cholesterol (Sigma, USA), 2.8 μL of 20 mM distearoyl-rac-glycerol-PEG2K (Avanti Polar Lipids, USA), and 0.7 μL of 20 mM 1,2-distearoyl-sn-glycero-3-phosphocholine (Sigma, USA) in CHCl₃, followed by a minor bath sonication. CHCl₃ was then removed by reduced pressure, and CaP-L was formed by rehydration with 100 μL of PBS 1× (pH=7.2). siRNA-CaP-L hydrodynamic size were measured with Zetasizer (ZS-90/Malvern, Malvern, U.K.) (Table 5). The material was able to knock down luciferase (as a model protein) in human cells (FIG. 9A) and was found to be safe to the cells (FIG. 9B).

Synthesis of siRNA/CpG Loaded Lipid-Coated CaP-L (siRNA/CpG-CaP-L)

A siRNA loaded calcium phosphate nanoparticle (siRNA-CaPNP) was synthesized in a water-in-oil microemulsion. Briefly, 5 μg of siRNA (Scramble siRNA) was added to 60 μL of 2.5 M CaCl₂) (Fisher Scientific, USA). The above solution was subsequently dispersed in 4 ml of cyclohexane (Sigma, USA)/Igepal CO-520 (Sigma, USA) (71:29, v/v) to form a calcium phase. 60 μL of 12.5 mM Na₂HPO₄ (Fisher Scientific, USA) was dispersed in another 4 mL of cyclohexane/Igepal CO-520 (71:29, v/v) to form the phosphate phase. 40 μL of 20 mM 1,2-dioleoyl-sn-glycero-3-phosphoethanolamine (Avanti Polar Lipids, USA) in CHCl₃ (Fisher Scientific, USA) were added to the phosphate phase. The phosphate phase was then added into the calcium phase dropwise, and the mixture was stirred for 10 minutes at room temperature. An equal volume of ethanol (Decon Labs, USA) was added to break the microemulsion, and the siRNA-CaPNP thus obtained was collected with centrifugation at 21,000 g for 15 minutes and then washed 3 times with absolute ethanol to remove the residual oil phase. The resulting pellets were suspended in 100 μL of CHCl₃. The siRNA/CaP-L was formed by mixing 10 μL of siRNA-CaPNP in CHCl₃ with 1.4 μL of 20 mM dimethyldioctadecylammonium bromide (Sigma, USA), 1.4 μL of 20 mM cholesterol (Sigma, USA), 2.8 μL of 20 mM distearoyl-rac-glycerol-PEG2K (Avanti Polar Lipids, USA), and 0.7 μL of 20 mM 1,2-distearoyl-sn-glycero-3-phosphocholine (Sigma, USA) in CHCl₃, following by a minor bath sonication. CHCl₃ was then removed by reduced pressure, and siRNA/CpG-CaP-L was formed by rehydration with 100 μL of PBS 1× (pH=7.2), which contained 2 μg of CpG (CpG 7909/2006).

Synthesis of mRNA/siRNA/CpG Loaded Lipid-Coated CaPNP (mRNA/siRNA/CpG-CaP-L)

A mRNA/siRNA loaded calcium phosphate core (mRNA/siRNA-CaPNP) was synthesized in a water-in-oil micro emulsion. 2.5 μg of siRNA (Scramble siRNA) and 2.5 μg of mRNA (Firefly luciferase mRNA) was added to 60 μL of 2.5 M CaCl₂) (Fisher Scientific, USA). The above solution was subsequently dispersed in 4 mL of cyclohexane/Igepal CO-520 (Sigma, USA) (71:29, v/v) to form a calcium phase. 60 μL of 12.5 mM Na₂HPO₄ (Fisher Scientific, USA) was dispersed in another 4 mL of cyclohexane/Igepal CO-520 (71:29, v/v) to form a phosphate phase. 40 μL of 20 mM 1,2-dioleoyl-sn-glycero-3-phosphoethanolamine (Avanti Polar Lipids, USA) in CHCl₃ (Fisher Scientific, USA) were added to the phosphate phase. The phosphate phase was then added dropwise into the calcium phase. The mixture was stirred for 10 minutes at room temperature. An equal volume of ethanol (Decon Labs, USA) was added to break the microemulsion. The mRNA/siRNA-CaPNP was collected with centrifugation at 21,000 g for 15 minutes and then washed 3 times with absolute ethanol to remove residual oil phase. The pellets were suspended in 100 μL of CHCl₃. The mRNA/siRNA/CaP-L was formed by mixing 10 μL of mRNA/siRNA-CaPNP in CHCl₃ with 1.4 μL of 20 mM dimethyldioctadecylammonium bromide (Sigma, USA), 1.4 μL of 20 mM cholesterol (Sigma, USA), 2.8 μL of 20 mM distearoyl-rac-glycerol-PEG2K (Avanti Polar Lipids, USA), and 0.7 μL of 20 mM 1,2-distearoyl-sn-glycero-3-phosphocholine (Sigma, USA) in CHCl₃, following by a minor bath sonication. CHCl₃ was then removed under reduced pressure, and mRNA/siRNA/CpG-CaP-L was formed by rehydration with 100 μL of PBS 1× (pH=7.2), which contained 1 μg of CpG (CpG 7909/2006).

### Example 6. Immunogenic Construct for COVID-19 Vaccine (AIRISE-CoV)

AIRISE-CoV synthesis and characterization. AIRISE-CoV consists of mesoporous silica nanoparticle (MSNP, 50 nm) core coated with cross-linked polyethylenimine (PEI) and polyethylene glycol (PEG) polymer layers. SARS-CoV-2 spike glycoprotein antigen (S) is loaded by conjugation to PEG on exterior similar to antibody loading in our previous work (Ngamcherdtrakul et al., Advanced Functional Materials, 25(18):2646-2659, 2015 and U.S. Patent Application Publication No. 2017/0173169), followed by loading of CpG oligonucleotide and siRNA via electrostatic interaction with PEI and protected from enzymatic degradation by PEG layer. The bio-reducible cross-linking allows the use of small molecular weight PEI (10 kDa) to achieve efficacy of large molecular weight PEI (25 kDa) required for endosomal escape of siRNA or proteins to cytosol without toxicity. The nanoparticle platform has been used for cancer vaccine delivery owing to its versatility for loading and protecting multiple types of cargos (siRNA, CpG) while maintaining small particle size (˜200 nm). In the cancer vaccine application, we do not load antigens on the constructs but rely on antigens inside the tumors (via intratumoral injection) to create the vaccine effect. The final composition of AIRISE-CoV is 15% PEI, 10% PEG (by TGA), 3% spike protein (by Bicinchoninic acid assay (BCA)) (all by weight of NP). CpG and siRNA are loaded at 4% and 2%, respectively, by weight of NP (complete loading confirmed by nanodrop).

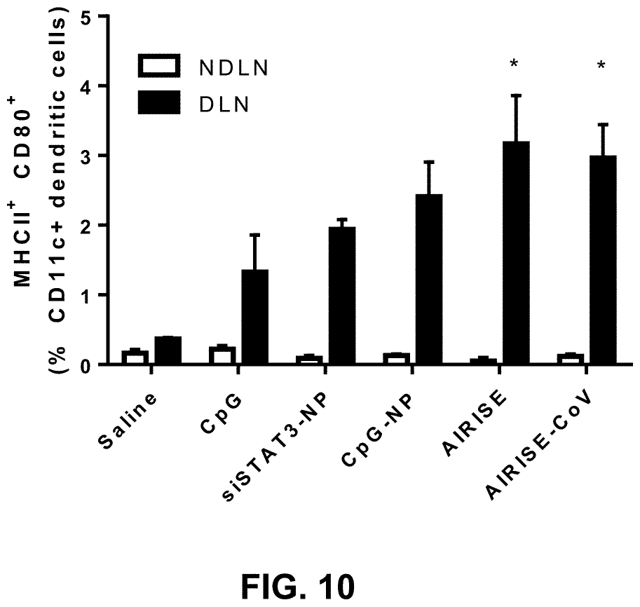

siSTAT3 and CpG on the nanoparticle promotes DC activation effectively. We show in FIG. 10 that, upon footpad injection, NP loaded with both siSTAT3 and CpG can activate DC in the draining lymph nodes (DLN) more effectively than NP delivering single component. Only AIRISE-treated mice show significantly higher proportion of activated DC in the draining lymph node when compared to saline (p<0.05). There appears to be no activity in the non-draining lymph node (NDLN), suggesting localized effect of DC priming. The antibody staining method to quantify a population of immune cells follows a published report (Ngamcherdtrakul et al., Advanced Material, 2021; doi: 10.1002/adma.202100628).

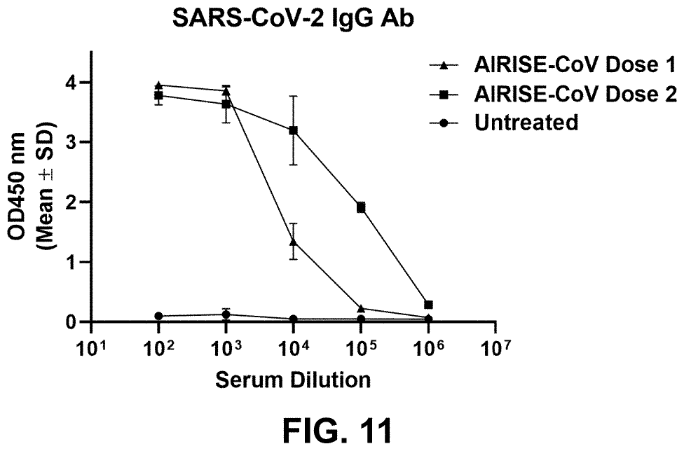

Presence of SARS-CoV-2 IgG antibodies in mice vaccinated with AIRISE-CoV. As shown in FIG. 11, BALB/c mice vaccinated with AIRISE-CoV generated high levels of SARS-CoV-2 binding IgG antibodies in serum, as assessed by end-point titer in ELISA. End-point titer represents the number of fold serum dilutions where SARS-CoV-2 binding antibodies are detectable and is widely used to assess induction of humoral immunogenicity (antibody production). AIRISE-CoV elicited end-point titers of 10⁵ and 10⁶ after 1 and 2 doses (FIG. 11), respectively, which is comparable to or better than other leading COVID-19 vaccine candidates (Table 6).

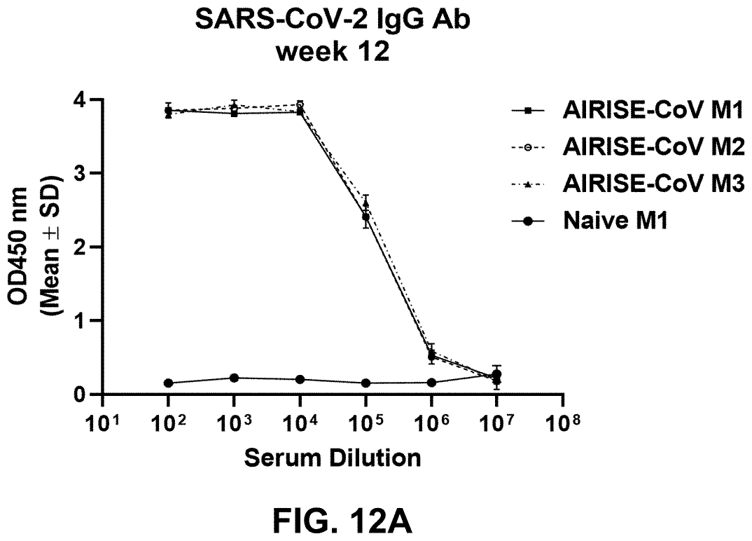

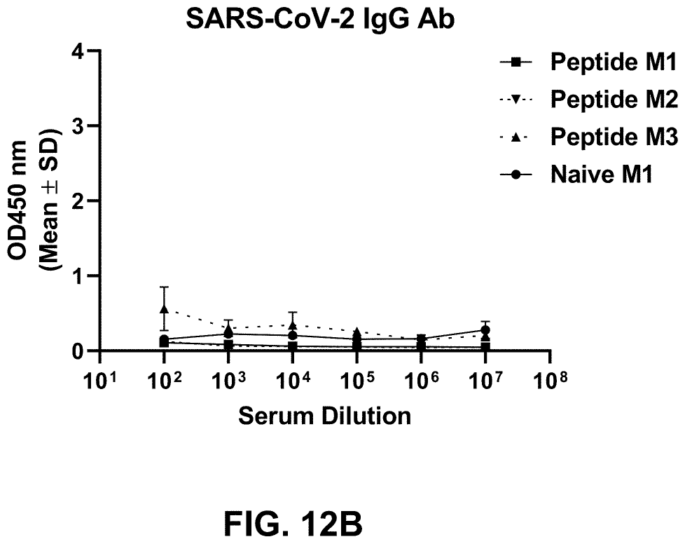

In FIG. 12A, we show that high levels of IgG antibodies were maintained in all immunized mice for up to 12 weeks after first dose. In addition, we show that replacing the full-length spike protein antigen with two immunogenic spike peptides (424-433 and 891-906, from JPT Peptide Technologies) did not elicit significant antibody titers (FIG. 12B), providing strong rationale for the use of full-length spike protein antigen. Both peptides were loaded on the nanoconstruct via electrostatic interactions. Without being bound to any explanation, the two peptides did not elicit a strong response. However, the same NP was shown previously for effective peptide delivery to trigger antigen specific immune response (e.g., SF delivery, FIG. 7).

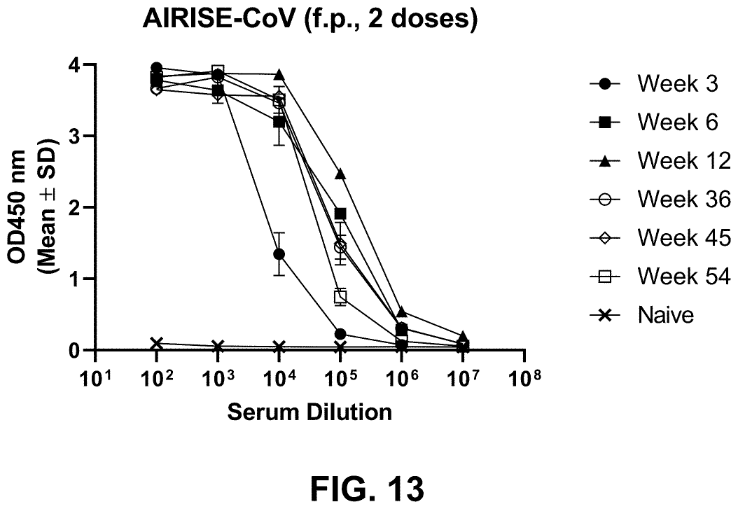

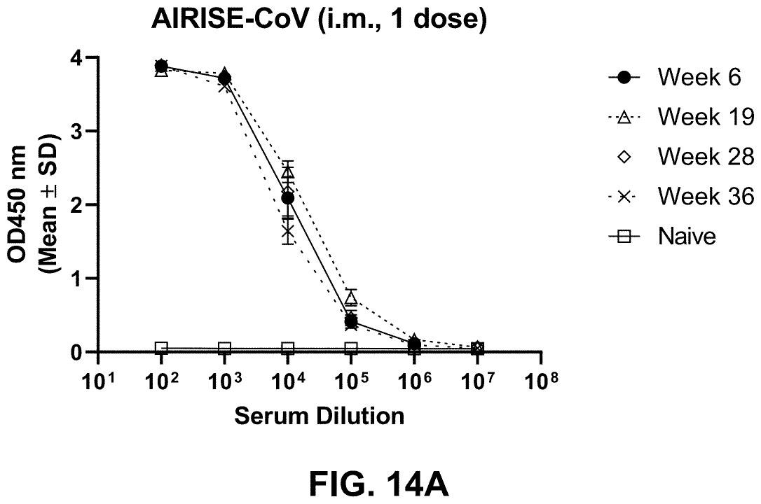

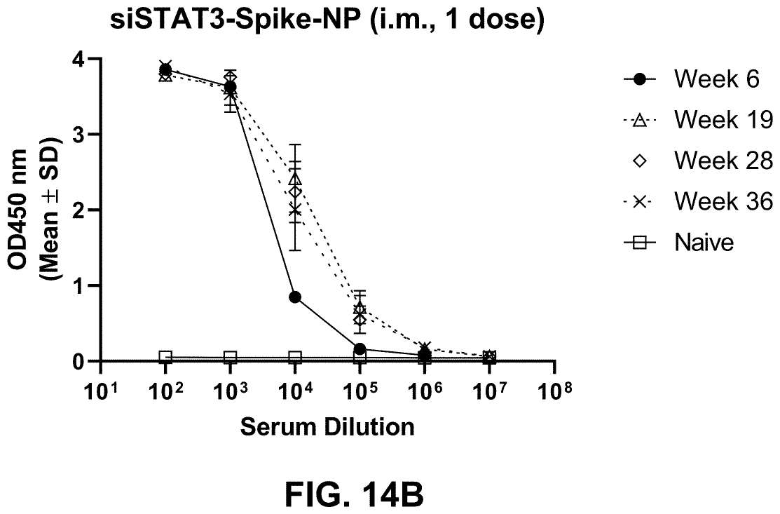

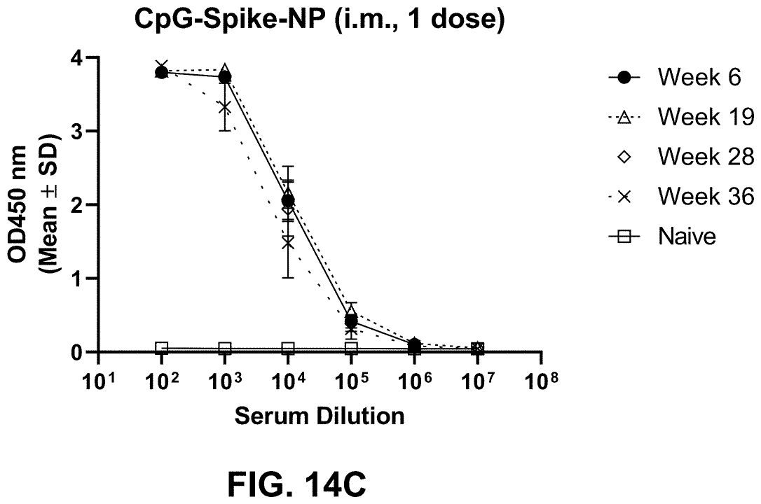

We found that mice injected with AIRISE-CoV via footpad (FIG. 13, 2 doses total) or intramuscularly (FIG. 14A, one dose only) had sustained high levels of the antibody to date (i.e., up to 54 weeks via footpad administration route, and 36 weeks via intramuscular route). This suggests that antibody generation and induction of humoral immunity are long-lasting.

TABLE 6
Antibody (Ab) response of leading COVID-19
vaccines or vaccine candidates

| | | Ab titer | Ab titer |
|---|---|---|---|
| | | in humans | in mice |
| Name | | (vs. | (End-point |
| (Company) | Vaccine Type | convalescent) | titer) |
| mRNA-1273 | mRNA encoding | 5.5-fold | ~10⁵ |
| (Moderna) | spike (S) protein | | |
| ChAdOx1 nCoV-19 | Adenovirus vector | Similar | ~10²-10³ |
| (AstraZeneca/ | expressing S protein | | |
| Oxford) | | | |
| BNT162b1 | mRNA encoding | ~2 to 5-fold | N/A |
| (Pfizer/BioNTech) | trimerized S-RBD | | |
| NVX-COV2373 | Recombinant NP with | 6-fold | ~10⁵ |
| (Novavax) | trimeric S protein | | |
| INO-4800 | DNA vaccine | N/A | ~10³ |
| (Inovio) | encoding S protein | | |

AIRISE-CoV with Spike protein, siSTAT3 and CpG were developed for effective vaccination in immunosuppressive or immune-compromised subjects (e.g., aged subjects or subjects with diseases and conditions that cause their immune system to be compromised). However, in the normal immune mice tested, we found that the mesoporous silica nanoparticles coated with PEI and PEG (NP) loaded with spike protein alone, spike protein and siSTAT3 (FIG. 14B), or spike protein and CpG (FIG. 14C) could also generate high and lasting antibody levels in these mice, suggesting that the NPs can be used effectively for antigen delivery to create vaccines better than free antigens (without np). As shown in FIGS. 14B-14C, vaccination with one dose of siSTAT3 loaded Spike-NP (FIG. 14B) or one dose of CpG loaded Spike-NP (FIG. 14C) could also elicit high levels of SARS-CoV-2 IgG antibodies that persist for at least 36 weeks. This further demonstrates that antibody generation and induction of humoral immunity are long-lasting. This may be due to the ability of NP to protect and retain the cargos and deliver them effectively to antigen presenting cells.

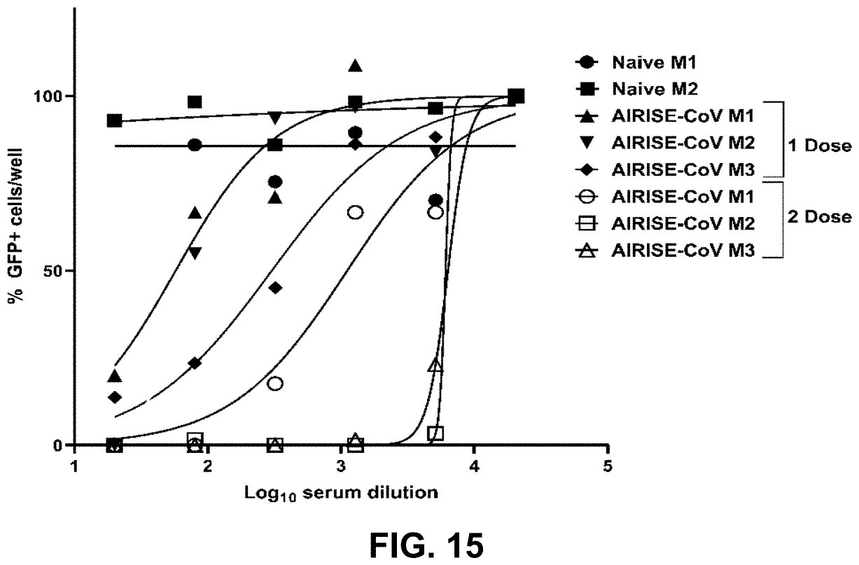

Neutralization of SARS-CoV-2 pseudo-virus by immunized sera. To determine if SARS-CoV-2 specific IgG antibodies are effective in neutralizing SARS-CoV-2, we constructed a pseudo-virus (CoV2-S-PsV) neutralization assay, which is a widely used method that assesses the ability of immunized serum to inhibit SARS-CoV-2 pseudo-virus from transfecting HEK293-derived cell line that expresses human ACE2 (spike protein binding receptor). The assay utilizes replication-defective, GFP-encoding reporter lentiviruses pseudo-typed with the SARS-CoV-2 spike (S) protein. Thus, neutralizing ability is determined by % GFP+ cells (i.e. low % GFP+ suggests inability of pseudo-virus to transfect HEK293-hACE2 cells due to presence of neutralizing antibodies). FIG. 15 shows how antibody titers in sera obtained from mice vaccinated with AIRISE-CoV can effectively neutralize CoV2-S-PsV infection (i.e. inhibit binding to ACE2 receptor), while sera from naïve mice had no effect. Furthermore, we found that neutralizing titers after two doses (dilution required to neutralize 50% of virus; NT₅₀) were higher than or comparable to those found in convalescent patient sera as shown in Table 7.

Table 7 shows neutralizing titers (dilution required to neutralize 50% of virus; NT₅₀) from samples in FIG. 15.

TABLE 7

| Serum Sample | NT₅₀ |
|---|---|
| Convalescent sample 1 | 3.3 × 10² |
| Convalescent sample 2 | 2.2 × 10³ |
| AIRISE-CoV M1 (1 dose) | 5.74 × 10¹ |
| AIRISE-CoV M2 (1 dose) | 7.57 × 10¹ |
| AIRISE-CoV M3 (1 dose) | 3.02 × 10² |
| AIRISE-CoV M1 (2 dose) | 1.11 × 10³ |
| AIRISE-CoV M2 (2 dose) | 6.09 × 10³ |
| AIRISE-CoV M3 (2 dose) | 6.34 × 10³ |

### Example 7. Nanoparticles with Crosslinked PEI and PEG (NP) are Safe to Antigen-Presenting Cells

As shown in FIG. 16, the herein-described NP loaded with siSTAT3 or siSTAT3+CpG were safe to both bone marrow derived dendritic cells (BMDC, FIG. 16A) and macrophages (J774, FIG. 16B). NP dose was 35 μg/ml (2 wt. % siRNA; 7 wt. % CpG). Viability was evaluated by CellTiter-Glo assay at 2 days post-treatment, following manufacturer's protocol.

### Example 8. Synthesis of mRNA Loaded MSNP Constructs

Mesoporous silica coated crosslinked PEI (MSNP-PEI) or crosslinked PEI and PEG (MSNP-PEI-PEG) can be used for mRNA delivery to generate vaccines. MSNP-PEI or MSNP-PEI-PEG was mixed with Firefly Luciferase mRNA (1 wt. % of the nanoparticles) in PBS on an orbital shaker at 350 rpm for 15-60 minutes at room temperature. The mRNA bound electrostatically to the external surface of the particles. Particle sizes before and after mixing with mRNA were measured using Zetasizer (Table 8). The supernatant containing unbound mRNA was obtained after centrifuging the mixture. mRNA concentration in supernatant was analyzed using NanoDrop (ND-1000, Thermo Scientific). Over 97% of mRNA was bound to MSNP-PEI or MSNP-PEI-PEG.

Table 8 shows the hydrodynamic size of nanoparticles loaded with mRNA.

TABLE 8

| Material | Z-Ave (nm) | PDI |
|---|---|---|
| MSNP-PEI | 100.7 ± 0.5 | 0.18 ± 0.01 |
| MSNP-PEI-PEG | 102.9 ± 1.8 | 0.16 ± 0.02 |
| MSNP-PEI + 1 wt. % mRNA | 222.9 ± 5.4 | 0.30 ± 0.04 |
| MSNP-PEI-PEG + 1 wt. % mRNA | 157.9 ± 2.2 | 0.35 ± 0.02 |

### XV. Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant change in activity (e.g., immunogenicity) of a nanoparticle construct/platform as described.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials is individually incorporated herein by reference in their entirety for their referenced teaching(s), as of the filing date of the first application in the priority chain in which the specific reference was included. For instance, with regard to chemical compounds and nucleic acid or amino acids sequences referenced herein that are available in a public database, the information in the cited database entry is incorporated herein by reference as of the date that the database identifier was first included in the text of an application in the priority chain.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8th Edition (Ed. J. Law & R. Rennie, Oxford University Press, 2020).

We claim:

1. An immunogenic construct comprising:
a nanoparticle platform (NP), comprising:
a mesoporous silica nanoparticle (MSNP); and
bound to the NP:
coronavirus antigen(s) comprising:
a full-length coronavirus spike glycoprotein, or
at least one protein subunit of a coronavirus spike glycoprotein; and
an adjuvant comprising a CpG oligonucleotide.

2. The immunogenic construct of claim 1, wherein the at least one subunit of the coronavirus spike glycoprotein comprises one or more of:
spike glycoprotein S1 region,
spike glycoprotein S2 region, or
spike glycoprotein Receptor Binding Domain (RBD) region.

3. The immunogenic construct of claim 1, wherein the coronavirus spike protein is a SARS-CoV-1, SARS-CoV-2, or MERS-CoV spike protein.

4. The immunogenic construct of claim 1, wherein the coronavirus antigen(s) bound to the NP comprise:
full-length coronavirus spike glycoprotein; and
at least one protein subunit of a coronavirus spike glycoprotein.

5. The immunogenic construct of claim 1, wherein the MSNP is functionalized with one or more thiol, amine, carboxylate, or phosphonate moieties.

6. The immunogenic construct of claim 1, further comprising one or more of a DNA TLR agonist containing a CpG sequence, a non-CpG DNA TLR agonist, an RNA TLR agonist, an aluminum salt, an anti-CD40 antibody, a fusion protein, a cytokine, a small molecule TLR agonist, an oil- or surfactant-based adjuvant, a lipopolysaccharide, a plant extract, or a derivative thereof.

7. The immunogenic construct of claim 1, wherein the CpG oligonucleotide is selected from the group consisting of CpG ODN 7909, CpG ODN 1826, CpG ODN D19, CpG ODN 1585, CpG ODN 2216, CpG ODN 2336, ODN 1668, ODN 1826, ODN 2006, ODN 2007, ODN 2395, ODN M362, and SD-10.

8. The immunogenic construct of claim 1, wherein the CpG oligonucleotide is present at 1-20 wt. % of the NP.

9. The immunogenic construct of claim 1, wherein MSNP has an average pore size of 2-6 nm, 7 nm, or less than 7 nm.

10. The immunogenic construct of claim 1, wherein the NP further comprises:
a crosslinked cationic polymer bound to an exterior surface of the MSNP;
a stabilizer bound to the crosslinked cationic polymer or the exterior surface of the MSNP; or
both a crosslinked cationic polymer bound to an exterior surface of the MSNP and a stabilizer bound to the crosslinked cationic polymer or the exterior surface of the MSNP.

11. The immunogenic construct of claim 10, wherein the cationic polymer comprises PEI, chitosan, polypropyleneimine, polylysine, polyamidoamine, poly(allylamine), poly(diallyldimethylammonium chloride), poly(N-isopropyl acrylamide-co-acrylamide), poly(N-isopropyl acrylamide-co-acrylic acid), diethylaminoethyl-dextran, poly-(N-ethyl-vinylpyridinium bromide), poly(dimethylamino)ethyl methacrylate, poly(ethylene glycol)-co-poly(trimethylaminoethylmethacrylate chloride), or a mixture of two or more thereof.

12. The immunogenic construct of claim 11, wherein the cationic polymer is or comprises PEI.

13. The immunogenic construct of claim 10, wherein the cationic polymer has a molecular weight of about 0.8 kDa to about 25 kDa.

14. The immunogenic construct of claim 10, wherein the cationic polymer is present at 1-50 wt. % of the NP.

15. The immunogenic construct of claim 10, wherein the stabilizer comprises PEG, dextran, polysialic acid, hyaluronic acid, polyvinyl pyrrolidone, polyvinyl alcohol, polyacrylamide, or a mixture of two or more thereof.

16. The immunogenic construct of claim 15, wherein the stabilizer is PEG.

17. The immunogenic construct of claim 10, wherein the stabilizer has a molecular weight of about 1 kDa to about 20 kDa, or about 5 kDa.

18. The immunogenic construct of claim 10, wherein the stabilizer is present at 1-50 wt. %, about 10 to 30 wt. %, about 5 to 20 wt. %, about 15 wt. %, or about 20 wt. % of the NP.

19. The immunogenic construct of claim 1, wherein the coronavirus antigen is present at 0.5-20 wt. % of the NP.

20. The immunogenic construct of claim 1, having a hydrodynamic diameter of:
about 50 nm to about 200 nm; or
about 100 nm to about 500 nm; or
about 200 nm to about 750 nm.

21. The immunogenic construct of claim 1, comprising:
an amount of crosslinked cationic polymer comprising polyethylenimine (PEI) bound electrostatically to an exterior surface of the MSNP, and wherein the PEI content is at least 10% by weight of the NP; and
an amount of a stabilizer comprising polyethylene glycol (PEG) bound covalently to the crosslinked PEI and/or the MSNP;
wherein the hydrodynamic size of the immunogenic construct is no more than 1 micron.

22. An immunogenic composition comprising a plurality of the immunogenic constructs of claim 1.

23. A composition comprising:
the immunogenic construct of claim 1, and
at least one biologically or pharmaceutically acceptable excipient.

24. A vaccine comprising:
the immunogenic construct of claim 1, and
a pharmaceutically acceptable excipient.

25. A method comprising administering to a subject an immune-stimulatory amount of the immunogenic construct of claim 1.

26. The method of claim 25, which induces an immune response against the coronavirus in the subject.

27. The method of claim 25, which treats or prevents a coronavirus infection in the subject.

28. The method of claim 27, wherein the subject is a human.

29. The method of claim 27, wherein the subject is immunocompromised.

30. The method of claim 27, wherein the immunogenic construct is administered transdermally, intramuscularly, by inhalation, or intranasally.


SEQUENCE LISTING (91 sequences)

SEQ ID NO: 1 - protein, 10 aa, SARS-CoV-2
KLPDDFTGCV

SEQ ID NO: 2 - protein, 15 aa, SARS-CoV-2
SQSIIAYTMSLGAEN

SEQ ID NO: 3 - protein, 15 aa, SARS-CoV-2
IPTNFTISVTTEILP

SEQ ID NO: 4 - protein, 23 aa, SARS-CoV-2
FGAGAALQIPFAMQMAYRFNGIG

SEQ ID NO: 5 - protein, 15 aa, SARS-CoV-2
APHGVVFLHVTYVPA

SEQ ID NO: 6 - protein, 15 aa, SARS-CoV-2
ATKAYNVTQAFGRRG

SEQ ID NO: 7 - protein, 15 aa, SARS-CoV-2
IASFRLFARTRSMWS

SEQ ID NO: 8 - protein, 15 aa, SARS-CoV-2
VKPSFYVYSRVKNLN

SEQ ID NO: 9 - RNA, 19 nt, Homo sapiens
ggaucuagaacagaaaaug

SEQ ID NO: 10 - RNA, 19 nt, Homo sapiens
cauuuucuguucuagaucc

SEQ ID NO: 11 - RNA, 19 nt, Mus musculus
cccacauaaaaaacaguug

SEQ ID NO: 12 - RNA, 19 nt, Mus musculus
caacuguuuuuuauguggg

SEQ ID NO: 13 - RNA, 25 nt, Homo sapiens
ccuacuggcauuugcugaacgcauu

SEQ ID NO: 14 - RNA, 25 nt, Homo sapiens
aaugcguucagcaaaugccaguagg

SEQ ID NO: 15 - RNA, 25 nt, Homo sapiens
cccuccugcaucauugcuuucauuu

SEQ ID NO: 16 - RNA, 25 nt, Homo sapiens
aaaugaaagcaaugaugcaggaggg

SEQ ID NO: 17 - RNA, 19 nt, Mus musculus
ccucugaaguucuaauuaa

SEQ ID NO: 18 - RNA, 21 nt, Mus musculus
ucggagacuucaagauuaauu

SEQ ID NO: 19 - RNA, 19 nt, Mus musculus
gggcuucuuccucgucucu

SEQ ID NO: 20 - RNA, 19 nt, Mus musculus
agagacgaggaagaagccc

SEQ ID NO: 21 - RNA, 19 nt, Homo sapiens
ccgugaguuuguccuuuca

SEQ ID NO: 22 - RNA, 19 nt, Homo sapiens
ugaaaggacaaacucacgg

SEQ ID NO: 23 - RNA, 17 nt, Homo sapiens
cacuuccgcacauuccg

SEQ ID NO: 24 - RNA, 19 nt, Homo sapiens
cugggaugccguguuauuu

SEQ ID NO: 25 - RNA, 21 nt, Homo sapiens
acuaccugagcuccuuccccu

SEQ ID NO: 26 - RNA, 21 nt, Homo sapiens
gcauccgcgugcacuuucauu

SEQ ID NO: 27 - RNA, 21 nt, Homo sapiens
aaugaaagugcacgcggaugc

SEQ ID NO: 28 - RNA, 23 nt, Mus musculus
gagcccuccucguccucgucuuc

SEQ ID NO: 29 - RNA, 23 nt, Mus musculus
gaagacgaggacgaggagggcuc

SEQ ID NO: 30 - RNA, 21 nt, Mus musculus
aaucaaguguggagcaacauu

SEQ ID NO: 31 - RNA, 21 nt, Mus musculus
uguugcuccacacuugauuuu

SEQ ID NO: 32 - RNA, 25 nt, Homo sapiens
gacaucuaugcaaugggcuuaguau

SEQ ID NO: 33 - RNA, 25 nt, Homo sapiens
gcaucucacucauguugauggucua

SEQ ID NO: 34 - RNA, 25 nt, Homo sapiens
aguaagacaugauucagccacagau

SEQ ID NO: 35 - RNA, 19 nt, Mus musculus
agaauaucuuggugaagaa

SEQ ID NO: 36 - RNA, 19 nt, Mus musculus
uucuucaccaagauauucu

SEQ ID NO: 37 - RNA, 19 nt, Homo sapiens
gaccucaagagcuccaaua

SEQ ID NO: 38 - RNA, 19 nt, Homo sapiens
uauuggagcucuugagguc

SEQ ID NO: 39 - RNA, 19 nt, Homo sapiens
cgacaugauagucacugac

SEQ ID NO: 40 - RNA, 19 nt, Homo sapiens
gucagugacuaucaugucg

SEQ ID NO: 41 - RNA, 21 nt, Mus musculus
aauaagcuccaagagaaaggc

SEQ ID NO: 42 - RNA, 18 nt, Homo sapiens
uuaauaagcuccagagaa

SEQ ID NO: 43 - RNA, 19 nt, Homo sapiens
uggaggacuuuaaggguua

SEQ ID NO: 44 - RNA, 19 nt, Homo sapiens
cauagaagccuacaugaca

SEQ ID NO: 45 - RNA, 19 nt, Homo sapiens
ucacggcgcugucaucgau

SEQ ID NO: 46 - RNA, 19 nt, Mus musculus
ccugagcaucuuagucaua

SEQ ID NO: 47 - RNA, 19 nt, Mus musculus
uaugacuaagaugcucagg

SEQ ID NO: 48 - RNA, 19 nt, Homo sapiens
ggacuggacccaucuuuca

SEQ ID NO: 49 - RNA, 19 nt, Homo sapiens
gggcggagaccacaguuug

SEQ ID NO: 50 - RNA, 19 nt, Homo sapiens
gggcuacucucaggauuag

SEQ ID NO: 51 - RNA, 19 nt, Homo sapiens
gaaguaacucgaacaguga

SEQ ID NO: 52 - RNA, 21 nt, Homo sapiens
cgccaacaccaucgugugcaa

SEQ ID NO: 53 - RNA, 21 nt, Homo sapiens
cgccagcaaccugaaucucaa

SEQ ID NO: 54 - RNA, 19 nt, Mus musculus
accugugccuacacuucaa

SEQ ID NO: 55 - RNA, 19 nt, Homo sapiens
ccucgcaugcugaucacaa

SEQ ID NO: 56 - RNA, 19 nt, Mus musculus
gauguugccuuccacuuua

SEQ ID NO: 57 - RNA, 19 nt, Mus musculus
uaaaguggaaggcaacauc

SEQ ID NO: 58 - RNA, 19 nt, Mus musculus
gcagacagcuuuucgcuua

SEQ ID NO: 59 - RNA, 19 nt, Mus musculus
uaagcgaaaagcugucugc

SEQ ID NO: 60 - RNA, 19 nt, Mus musculus
ggucaacgaugcucaccua

SEQ ID NO: 61 - RNA, 19 nt, Mus musculus
uaggugagcaucguugacc

SEQ ID NO: 62 - RNA, 66 nt, Homo sapiens
gaucccguaugagcguuaggacacuuugauauccgaguguccuaacgcucaauacuuuuuuccaaa

SEQ ID NO: 63 - RNA, 65 nt, Homo sapiens
ggcauacucgcaauccugugaaacuauaggcucacaggauugcgaguaugaaaaaagguuuucga

SEQ ID NO: 64 - RNA, 19 nt, Mus musculus
agucggaggcuuaauuaca

SEQ ID NO: 65 - RNA, 19 nt, Mus musculus
caggaaauuugccuauuga

SEQ ID NO: 66 - RNA, 19 nt, Mus musculus
uaaggaccaagaccaucca

SEQ ID NO: 67 - RNA, 25 nt, Homo sapiens
gggagacacuccaucacagucacua

SEQ ID NO: 68 - RNA, 25 nt, Homo sapiens
uagugacugugauggagugucuccc

SEQ ID NO: 69 - RNA, 19 nt, Homo sapiens
cggcaugugaggaucaaaa

SEQ ID NO: 70 - RNA, 19 nt, Homo sapiens
uuuugauccucacaugccg

SEQ ID NO: 71 - RNA, 19 nt, Homo sapiens
gcgggaugacuuuccaaga

SEQ ID NO: 72 - RNA, 19 nt, Homo sapiens
ucuuggaaagucaucccgc

SEQ ID NO: 73 - RNA, 19 nt, Homo sapiens
gcuaucgaauuugucaacc

SEQ ID NO: 74 - RNA, 18 nt, Homo sapiens
gguugacaaauucgauag

SEQ ID NO: 75 - RNA, 25 nt, Mus musculus
uuucaaagaccucuggaucuugacc

SEQ ID NO: 76 - RNA, 19 nt, Mus musculus
ugccuacgaacucuucacc

SEQ ID NO: 77 - RNA, 19 nt, Mus musculus
ggugaagaguucguaggca

SEQ ID NO: 78 - RNA, 19 nt, Homo sapiens
gcuucguggucgacuucau

SEQ ID NO: 79 - RNA, 19 nt, Mus musculus
uauggagcugcagaggaug

SEQ ID NO: 80 - RNA, 19 nt, Mus musculus
cauccucugcagcuccaua

SEQ ID NO: 81 - RNA, 19 nt, Homo sapiens
ccaucaugggcuggacauu

SEQ ID NO: 82 - RNA, 19 nt, Homo sapiens
gcaaccuucugauguaagu

SEQ ID NO: 83 - RNA, 19 nt, Homo sapiens
cuaccucccuacagacaga

SEQ ID NO: 84 - RNA, 21 nt, Mus musculus
ggguguuugcaaaugauuauu

SEQ ID NO: 85 - RNA, 21 nt, Mus musculus
uaaucauuugcaaacacccuu

SEQ ID NO: 86 - RNA, 25 nt, Homo sapiens
ccaauggcuaagugaagaugacaau

SEQ ID NO: 87 - RNA, 25 nt, Homo sapiens
ccacacgacgggaagacaaguucau

SEQ ID NO: 88 - RNA, 19 nt, Mus musculus
gguguauacaggaacaaua

SEQ ID NO: 89 - RNA, 19 nt, Mus musculus
uauuguuccuguauacacc

SEQ ID NO: 90 - protein, 8 aa, Gallus gallus
SIINFEKL

SEQ ID NO: 91 - RNA, 18 nt, Homo sapiens
guauuggagacgauuuaa